SEQUENCE LISTING (82 sequences)

SEQ ID NO: 1 - DNA, 27 bp, Artificial Sequence
  KanColE1_1F
gggccaaggatctgatggcgcagggga

SEQ ID NO: 2 - DNA, 40 bp, Artificial Sequence
  KanColE1_1R
cttggccgcagcggccgagcaaaaggccagcaaaaggcca

SEQ ID NO: 3 - DNA, 33 bp, Artificial Sequence
  KanColE1_2F
cccggatccgcagtgggcttacatggcgatagc

SEQ ID NO: 4 - DNA, 38 bp, Artificial Sequence
  KanColE1_2R
cccgtatacatcgatttaattaagagcaaaaggccagc

SEQ ID NO: 5 - DNA, 34 bp, Artificial Sequence
  Cla_delE1_F
gggatcgatttaagggtgggaaagaatatataag

SEQ ID NO: 6 - DNA, 33 bp, Artificial Sequence
  BstZ_R
cccgtatacggggacacggacagccttttcgtc

SEQ ID NO: 7 - DNA, 32 bp, Artificial Sequence
  KanColE1_3F
ttaattaagcagtgggcttacatggcgatagc

SEQ ID NO: 8 - DNA, 38 bp, Artificial Sequence
  KanColE1_3R
cccggatccatcgatttaattaagagcaaaaggccagc

SEQ ID NO: 9 - DNA, 34 bp, Artificial Sequence
  E3_Spe_F
gggactagtttcgcgccctttctcaaatttaagc

SEQ ID NO: 10 - DNA, 35 bp, Artificial Sequence
  delE3_R
gcggatggacaggaacttataacattcagtcgtag

SEQ ID NO: 11 - DNA, 35 bp, Artificial Sequence
  delE3_F
ctacgactgaatgttataagttcctgtccatccgc

SEQ ID NO: 12 - DNA, 34 bp, Artificial Sequence
  E3_Nde_R
gggcatatggatacacggggttgaaggtatcttc

SEQ ID NO: 13 - DNA, 17 bp, Artificial Sequence
  Cla_EcoR_S
cgatccggaggcccttg

SEQ ID NO: 14 - DNA, 19 bp, Artificial Sequence
  Cla_EcoR_AS
aattcaagggcctccggat

SEQ ID NO: 15 - DNA, 22 bp, Artificial Sequence
  Avr_F
ctgcctaggcaaaatagcaccc

SEQ ID NO: 16 - DNA, 35 bp, Artificial Sequence
  Avr_dITR_R
aacgatgtaagttttagggcggagtaacttgtatg

SEQ ID NO: 17 - DNA, 35 bp, Artificial Sequence
  Avr_dITR_F
gccctaaaacttacatcgttaattaagcagtgggc

SEQ ID NO: 18 - DNA, 22 bp, Artificial Sequence
  Rsr_R
tagcggtccgccacacccagcc

SEQ ID NO: 19 - DNA, 36 bp, Artificial Sequence
  KanColE1_4F
ggttggcgcgccctacgtcacccgccccgttcccac

SEQ ID NO: 20 - DNA, 34 bp, Artificial Sequence
  KanColE1_4R
cccgagctcaaactacataagacccccaccttat

SEQ ID NO: 21 - DNA, 30 bp, Artificial Sequence
  Sac_Pst_Avr_Asc_S
cttaacctgcagatcctcctaggtttttgg

SEQ ID NO: 22 - DNA, 38 bp, Artificial Sequence
  Sac_Pst_Avr_Asc_AS
cgcgccaaaaacctaggaggatctgcaggttaagagct

SEQ ID NO: 23 - DNA, 55 bp, Artificial Sequence
  Sac_Hpa_Avr_S
cgggcggcgacctcgcgggttaaccgtcctttaaaaaagtcgtttctgcaagctc

SEQ ID NO: 24 - DNA, 63 bp, Artificial Sequence
  Sac_Hpa_Avr_AS
ctaggagcttgcagaaacgacttttttaaaggacggttaacccgcgaggtcgccgcccgagct

SEQ ID NO: 25 - DNA, 35 bp, Artificial Sequence
  SacI/ApaI_S
catggttccaaaatgccccttaaccgggttgggcc

SEQ ID NO: 26 - DNA, 35 bp, Artificial Sequence
  SacI/ApaI_AS
caacccggttaaggggcattttggaaccatgagct

SEQ ID NO: 27 - DNA, 31 bp, Artificial Sequence
  ApaI/AvrII_S
catggttccaaaatgccccttaaccgggttc

SEQ ID NO: 28 - DNA, 39 bp, Artificial Sequence
  ApaI/AvrII_AS
ctaggaacccggttaaggggcattttggaaccatgggcc

SEQ ID NO: 29 - DNA, 33 bp, Artificial Sequence
  pGLAdSMAR_F
tctgggcccaaataaacttataaattgtgagag

SEQ ID NO: 30 - DNA, 33 bp, Artificial Sequence
  pGLAdSMAR_R
cccatgcatatatttaaagaaaaaaaaattgta

SEQ ID NO: 31 - DNA, 23 bp, Artificial Sequence
  pCAG_F
gttattaatagtaatcaattacg

SEQ ID NO: 32 - DNA, 44 bp, Artificial Sequence
  pCAG_R
cttgggtctccctatcgcccgccgcgcgcttcgctttttatagg

SEQ ID NO: 33 - DNA, 38 bp, Artificial Sequence
  bGint_F
ggcgatagggagacccaagctggtgagtttggggaccc

SEQ ID NO: 34 - DNA, 43 bp, Artificial Sequence
  bGint_R
gggaagcttgggtcccctgtaggaaaaagaagaaggcatgaac

SEQ ID NO: 35 - DNA, 48 bp, Artificial Sequence
  Ad5pTP_F
gggaagcttaccatggccttgagcgtcaacgattgcgcgcgcctgacc

SEQ ID NO: 36 - DNA, 34 bp, Artificial Sequence
  Ad5pTP_R
ggcgaattcctaaaagcggtgacgcgggcgagcc

SEQ ID NO: 37 - DNA, 24 bp, Artificial Sequence
  Ad5Fiber_F
cgcgcaagaccgtctgaagatacc

SEQ ID NO: 38 - DNA, 23 bp, Artificial Sequence
  Ad5Fiber_R
ggcctgatgtttgcagggctagc

SEQ ID NO: 39 - DNA, 45 bp, Artificial Sequence
  N-F
tcttatgtagtttgagctcggctttggttatttgatggattgacc

SEQ ID NO: 40 - DNA, 43 bp, Artificial Sequence
  N-R
tccggaggggctcgaggctggcttaaaacagtaactcaatatg

SEQ ID NO: 41 - DNA, 43 bp, Artificial Sequence
  C-F
ctcgagcccctccggaggagtggccagggcgttctggaggtag

SEQ ID NO: 42 - DNA, 41 bp, Artificial Sequence
  C-R
cgccaaaaacctaggtgggcccaggaagactcactggttgg

SEQ ID NO: 43 - DNA, 52 bp, Artificial Sequence
  Nsi-Xho-S
tcgagctagactctggggctaaagctgcagtatccatcacactggcggccgc

SEQ ID NO: 44 - DNA, 60 bp, Artificial Sequence
  Nsi-Xho-AS
tcgagcggccgccagtgtgatggatactgcagctttagccccagagtctagctcgatgca

SEQ ID NO: 45 - DNA, 36 bp, Artificial Sequence
  F1-F
cctcctcgagctagactctggggctaaagcaatgag

SEQ ID NO: 46 - DNA, 43 bp, Artificial Sequence
  F1-R
cccacgcgtgcggccgcttgagaggcagaggcaaaggcaagtg

SEQ ID NO: 47 - DNA, 38 bp, Artificial Sequence
  F2-F
tcaagcggccgcgtgctgagactaaagggatgtgctac

SEQ ID NO: 48 - DNA, 35 bp, Artificial Sequence
  F2-R
cccgtcgaccatcattctaaggcctgcctgagcta

SEQ ID NO: 49 - DNA, 35 bp, Artificial Sequence
  F3-F
cccgtcgacgcactggctttacaggggccgtctgc

SEQ ID NO: 50 - DNA, 35 bp, Artificial Sequence
  F3-R
gggcgatcgggaaggaaacctacctagcctacaag

SEQ ID NO: 51 - DNA, 35 bp, Artificial Sequence
  F4-F
gggcgatcggtagactaggatagcctcaaactcct

SEQ ID NO: 52 - DNA, 35 bp, Artificial Sequence
  F4-R
gggacgcgtgttaacatactatcagaatagtgata

SEQ ID NO: 53 - DNA, 35 bp, Artificial Sequence
  F5-F
gggacgcgttgggtgcaatcttaccagagccttac

SEQ ID NO: 54 - DNA, 25 bp, Artificial Sequence
  F5-R
acggcagttcaaaatcaagtaatac

SEQ ID NO: 55 - DNA, 41 bp, Artificial Sequence
  SMAR-F
cggcctgggtggccaaataaacttataaattgtgagagaaa

SEQ ID NO: 56 - DNA, 35 bp, Artificial Sequence
  SMAR-R
caggtcgacatatttaaagaaaaaaaaattgtatc

SEQ ID NO: 57 - DNA, 103 bp, Artificial Sequence
  HTTmshR1_S
gatcctgctgttgacagtgagcgagaccgtgtgaatcattgtctatagtgaagccacagatgtatagacaatgattcacacggtcgtgcctactgcctcggag

SEQ ID NO: 58 - DNA, 103 bp, Artificial Sequence
  HTTmshR1_AS
aattctccgaggcagtaggcacgaccgtgtgaatcattgtctatacatctgtggcttcactatagacaatgattcacacggtctcgctcactgtcaacagcag

SEQ ID NO: 59 - DNA, 103 bp, Artificial Sequence
  HTTmshR2_S
gatcctgctgttgacagtgagcgacagcttgtccaggtttatgaatagtgaagccacagatgtattcataaacctggacaagctggtgcctactgcctcggag

SEQ ID NO: 60 - DNA, 103 bp, Artificial Sequence
  HTTmshR2_AS
aattctccgaggcagtaggcaccagcttgtccaggtttatgaatacatctgtggcttcactattcataaacctggacaagctgtcgctcactgtcaacagcag

SEQ ID NO: 61 - DNA, 103 bp, Artificial Sequence
  HTTmshR3_S
gatcctgctgttgacagtgagcgaggatacctgaaatcctgcttttagtgaagccacagatgtaaaagcaggatttcaggtatccgtgcctactgcctcggag

SEQ ID NO: 62 - DNA, 103 bp, Artificial Sequence
  HTTmshR3_AS
aattctccgaggcagtaggcacggatacctgaaatcctgcttttacatctgtggcttcactaaaagcaggatttcaggtatcctcgctcactgtcaacagcag

SEQ ID NO: 63 - DNA, 2905 bp, Artificial Sequence
  pGT2
gcgcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccactagtaacggccgccagtgtgctggaattccagcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgaggtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaa

SEQ ID NO: 64 - DNA, 3451 bp, Artificial Sequence
  pBest
ggccgctgcggccgtttaaacgccagcaacgcggcctttttacggttcctggccttttgctggccttttgctcttaattaaatcgcatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtcgagtgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggacccaagcttggtaccgagctcggatccactagtaacggccgccagtgtgctggaattctgcagatatccatcacactggcggccgctcgagcatgcatctagagggccctattcaattctttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcatcgatccataacttcgtataatgtatgctatacgaagttatccagatctcgatttaagggtgggaaagaatatataaggtgggggtcttatgtagtttaaacgctagcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc

SEQ ID NO: 65 - DNA, 15804 bp, Artificial Sequence
  3rd DNA fragment
gtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcc

SEQ ID NO: 66 - DNA, 2248 bp, Artificial Sequence
  4th DNA fragment
gggatcgatttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacggg

SEQ ID NO: 67 - DNA, 12506 bp, Artificial Sequence
  5th DNA fragment
ggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 68 - DNA, 884 bp, Artificial Sequence
  6th DNA fragment
gggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttataagttcctgtccatccgcacccactatcttcatgttgttgcagatgaagcgcgcaagaccgtctgaagataccttcaaccccgtgtatccatatgccc

SEQ ID NO: 69 - DNA, 18040 bp, Artificial Sequence
  DNA corresponding to the ClaI-BamHI region of Psi5-Left-Arm-2
atcgatttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcc

SEQ ID NO: 70 - DNA, 1238 bp, Artificial Sequence
  7th DNA fragment
ctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacttacatcgttaattaagcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgcta

SEQ ID NO: 71 - DNA, 30991 bp, Artificial Sequence
  pAdBest_dITR
ttaattaagcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcttaattaaatcgatttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttagttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacttacatcg

SEQ ID NO: 72 - DNA, 1995 bp, Artificial Sequence
  8th DNA fragment
ggttggcgcgccctacgtcacccgccccgttcccacgccccgcgccacgtcacaaactccaccccctcattatcatattggcttcaatccaaaataaggtatattattgatgatgttacatcgttaattaagcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcttaattaaatcgatttaagggtgggaaagaatatataaggtgggggtcttatgtagtttgagctcggg

SEQ ID NO: 73 - DNA, 26877 bp, Artificial Sequence
  pGLAd
ttaattaagcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcttaattaaatcgatttaagggtgggaaagaatatataaggtgggggtcttatgtagtttgagctcgggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgaggctttttggcctctgtcgtttcctttctctgtttttgtccgtggaatgaacaatggaagtcaacaaaaagcagctggctgacattttcggtgcgagtatccgtaccattcagaactggcaggaacagggaatgcccgttctgcgaggcggtggcaagggtaatgaggtgctttatgactctgccgccgtcataaaatggtatgccgaaagggatgctgaaattgagaacgaaaagctgcgccgggaggttgaagaactgcggcaggccagcgaggcagatctccagccaggaactattgagtacgaacgccatcgacttacgcgtgcgcaggccgacgcacaggaactgaagaatgccagagactccgctgaagtggtggaaaccgcattctgtactttcgtgctgtcgcggatcgcaggtgaaattgccagtattctcgacgggctccccctgtcggtgcagcggcgttttccggaactggaaaaccgacatgttgatttcctgaaacgggatatcatcaaagccatgaacaaagcagccgcgctggatgaactgataccggggttgctgagtgaatatatcgaacagtcaggttaacaggctgcggcattttgtccgcgccgggcttcgctcactgttcaggccggagccacagaccgccgttgaatgggcggatgctaattactatctcccgaaagaatccgcataccaggaagggcgctgggaaacactgccctttcagcgggccatcatgaatgcgatgggcagcgactacatccgtgaggtgaatgtggtgaagtctgcccgtgtcggttattccaaaatgctgctgggtgtttatgcctactttatagagcataagcagcgcaacacccttatctggttgccgacggatggtgatgccgagaactttatgaaaacccacgttgagccgactattcgtgatattccgtcgctgctggcgctggccccgtggtatggcaaaaagcaccgggataacacgctcaccatgaagcgtttcactaatgggcgtggcttctggtgcctgggcggtaaagcggcaaaaaactaccgtgaaaagtcggtggatgtggcgggttatgatgaacttgctgcttttgatgatgatattgaacaggaaggctctccgacgttcctgggtgacaagcgtattgaaggctcggtctggccaaagtccatccgtggctccacgccaaaagtgagaggcacctgtcagattgagcgtgcagccagtgaatccccgcattttatgcgttttcatgttgcctgcccgcattgcggggaggagcagtatcttaaatttggcgacaaagagacgccgtttggcctcaaatggacgccggatgacccctccagcgtgttttatctctgcgagcataatgcctgcgtcatccgccagcaggagctggactttactgatgcccgttatatctgcgaaaagaccgggatctggacccgtgatggcattctctggttttcgtcatccggtgaagagattgagccacctgacagtgtgacctttcacatctggacagcgtacagcccgttcaccacctgggtgcagattgtcaaagactggatgaaaacgaaaggggatacgggaaaacgtaaaaccttcgtaaacaccacgctcggtgagacgtgggaggcgaaaattggcgaacgtccggatgctgaagtgatggcagagcggaaagagcattattcagcgcccgttcctgaccgtgtggcttacctgaccgccggtatcgactcccagctggaccgctacgaaatgcgcgtatggggatgggggccgggtgaggaaagctggctgattgaccggcagattattatgggccgccacgacgatgaacagacgctgctgcgtgtggatgaggccatcaataaaacctatacccgccggaatggtgcagaaatgtcgatatcccgtatctgctgggatactggcgggattgacccgaccattgtgtatgaacgctcgaaaaaacatgggctgttccgggtgatccccattaaaggggcatccgtctacggaaagccggtggccagcatgccacgtaagcgaaacaaaaacggggtttaccttaccgaaatcggtacggataccgcgaaagagcagatttataaccgcttcacactgacgccggaaggggatgaaccgcttcccggtgccgttcacttcccgaataacccggatatttttgatctgaccgaagcgcagcagctgactgctgaagagcaggtcgaaaaatgggtggatggcaggaaaaaaatactgtgggacagcaaaaagcgacgcaatgaggcactcgactgcttcgtttatgcgctggcggcgctgcgcatcagtatttcccgctggcagctggatctcagtgcgctgctggcgagcctgcaggaagaggatggtgcagcaaccaacaagaaaacactggcagattacgcccgtgccttatccggagaggatgaatgacgcgacaggaagaacttgccgctgcccgtgcggcactgcatgacctgatgacaggtaaacgggtggcaacagtacagaaagacggacgaagggtggagtttacggccacttccgtgtctgacctgaaaaaatatattgcagagctggaagtgcagaccggcatgacacagcgacgcaggggacctgcaggattttatgtatgaaaacgcccaccattcccacccttctggggccggacggcatgacatcgctgcgcgaatatgccggttatcacggcggtggcagcggatttggagggcagttgcggtcgtggaacccaccgagtgaaagtgtggatgcagccctgttgcccaactttacccgtggcaatgcccgcgcagacgatctggtacgcaataacggctatgccgccaacgccatccagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcctcgttcattcacgtttttgaacccgtggaggacgggcagactcgcggtgcaaatgtgttttacagcgtgatggagcagatgaagatgctcgacacgctgcagaacacgcagctgcagagcgccattgtgaaggcgatgtatgccgccaccattgagagtgagctggatacgcagtcagcgatggattttattctgggcgcgaacagtcaggagcagcgggaaaggctgaccggctggattggtgaaattgccgcgtattacgccgcagcgccggtccggctgggaggcgcaaaagtaccgcacctgatgccgggtgactcactgaacctgcagacggctcaggatacggataacggctactccgtgtttgagcagtcactgctgcggtatatcgctgccgggctgggtgtctcgtatgagcagctttcccggaattacgcccagatgagctactccacggcacgggccagtgcgaacgagtcgtgggcgtactttatggggcggcgaaaattcgtcgcatcccgtcaggcgagccagatgtttctgtgctggctggaagaggccatcgttcgccgcgtggtgacgttaccttcaaaagcgcgcttcagttttcaggaagcccgcagtgcctgggggaactgcgactggataggctccggtcgtatggccatcgcgatggtctgaaagaagttcaggaagcggtgatgctgatagaagccggactgagtacctacgagaaagagtgcgcaaaacgcggtgacgactatcaggaaatttttgcccagcaggtccgtgaaacgatggagcgccgtgcagccggtcttaaaccgcccgcctgggcggctgcagcatttgaatccgggctgcgacaatcaacagaggaggagaagagtgacagcagagctgcgtaatctcccgcatattgccagcatggcctttaatgagccgctgatgcttgaacccgcctatgcgcgggttttcttttgtgcgcttgcaggccagcttgggatcagcagcctgacggatgcggtgtccggcgacagcctgactgcccaggaggcactcgcgacgctggcattatccggtgatgatgacggaccacgacaggcccgcagttatcaggtcatgaacggcatcgccgtgctgccggtgtccggcacgctggtcagccggacgcgggcgctgcagccgtactcggggatgaccggttacaacggcattatcgcccgtctgcaacaggctgccagcgatccgatggtggacggcattctgctcgatatggacacgcccggcgggatggtggcgggggcatttgactgcgctgacatcatcgcccgtgtgcgtgacataaaaccggtatgggcgcttgccaacgacatgaactgcagtgcaggtcagttgcttgccagtgccgcctcccggcgtctggtcacgcagaccgcccggacaggctccatcggcgtcatgatggctcacagtaattacggtgctgcgctggagaaacagggtgtggaaatcacgctgatttacagcggcagccataaggtggatggcaacccctacagccatcttccggatgacgtccgggagacactgcagtcccggatggacgcaacccgccagatgtttgcgcagaaggtgtcggcatataccggcctgtccgtgcaggttgtgctggataccgaggctgcagtgtacagcggtcaggaggccattgatgccggactggctgatgaacttgttaacagcaccgatgcgatcaccgtcatgcgtgatgcactggatgcacgtaaatcccgtctctcaggagggcgaatgaccaaagagactcaatcaacaactgtttcagccactgcttcgcaggctgacgttactgacgtggtgccagcgacggagggcgagaacgccagcgcggcgcagccggacgtgaacgcgcagatcaccgcagcggttgcggcagaaaacagccgcattatggggatcctcaactgtgaggaggctcacggacgcgaagaacaggcacgcgtgctggcagaaacccccggtatgaccgtgaaaacggcccgccgcattctggccgcagcaccacagagtgcacaggcgcgcagtgacactgcgctggatcgtctgatgcagggggcaccggcaccgctggctgcaggtaacccggcatctgatgccgttaacgatttgctgaacacaccagtgtaagggatgtttatgacgagcaaagaaacctttacccattaccagccgcagggcaacagtgacccggctcataccgcaaccgcgcccggcggattgagtgcgaaagcgcctgcaatgaccccgctgatgctggacacctccagccgtaagctggttgcgtgggatggcaccaccgacggtgctgccgttggcattcttgcggttgctgctgaccagaccagcaccacgctgacgttctacaagtccggcacgttccgttatgaggatgtgctctggccggaggctgccagcgacgagacgaaaaaacggaccgcgtttgccggaacggcaatcagcatcgtttaactttacccttcatcactaaaggccgcctgtgcggctttttttacgggatttttttatgtcgatgtacacaaccgcccaactgctggcggcaaatgagcagaaatttaagtttgatccgctgtttctgcgtctctttttccgtgagagctatcccttcaccacggagaaagtctatctctcacaaattccgggactggtaaacatggcgctgtacgtttcgccgattgtttccggtgaggttatccgttcccgtggcggctccacctctgaatttacgccgggatatgtcaagccgaagcatgaagtgaatccgcagatgaccctgcgtcgcctgccggatgaagatccgcagaatctggcggacccggcttaccgccgccgtcgcatcatcatgcagaacatgcgtgacgaagagctggccattgctcaggtcgaagagatgcaggcagtttctgccgtgcttaagggcaaatacaccatgaccggtgaagccttcgatccggttgaggtggatatgggccgcagtgaggagaataacatcacgcagtccggcggcacggagtggagcaagcgtgacaagtccacgtatgacccgaccgacgatatcgaagcctacgcgctgaacgccagcggtgtggtgaatatcatcgtgttcgatccgaaaggctgggcgctgttccgttccttcaaagccgtcaaggagaagctggatacccgtcgtggctctaattccgagctggagacagcggtgaaagacctgggcaaagcggtgtcctataaggggatgtatggcgatgtggccatcgtcgtgtattccggacagtacgtggaaaacggcgtcaaaaagaacttcctgccggacaacacgatggtgctggggaacactcaggcacgcggtctgcgcacctatggctgcattcaggatgcggacgcacagcgcgaaggcattaacgcctctgcccgttacccgaaaaactgggtgaccaccggcgatccggcgcgtgagttcaccatgattcagtcagcaccgctgatgctgctggctgaccctgatgagttcgtgtccgtacaactggcgtaatcatggcccttcggggccattgtttctctgtggaggagtccatgacgaaagatgaactgattgcccgtctccgctcgctgggtgaacaactgaaccgtgatgtcagcctgacggggacgaaagaagaactggcgctccgtgtggcagagctgaaagaggagcttgatgacacggatgaaactgccggtcaggacacccctctcagccgggaaaatgtgctgaccggacatgaaaatgaggtgggatcagcgcagccggataccgtgattctggatacgtctgaactggtcacggtcgtggcactggtgaagctgcatactgatgcacttcacgccacgcgggatgaacctgtggcatttgtgctgccgggaacggcgtttcgtgtctctgccggtgtggcagccgaaatgacagagcgcggcctggccagaatgcaataacgggaggcgctgtggctgatttcgataacctgttcgatgctgccattgcccgcgccgatgaaacgatacgcgggtacatgggaacgtcagccaccattacatccggtgagcagtcaggtgcggtgatacgtggtgtttttgatgaccctgaaaatatcagctatgccggacagggcgtgcgcgttgaaggctccagcccgtccctgtttgtccggactgatgaggtgcggcagctgcggcgtggagacacgctgaccatcggtgaggaaaatttctgggtagatcgggtttcgccggatgatggcggaagttgtcatctctggcttggacggggcgtaccgcctgccgttaaccgtcgccgctgaaagggggatgtatggccataaaaggtcttgagcaggccgttgaaaacctcagccgtatcagcaaaacggcggtgcctggtgccgccgcaatggccattaaccgcgttgcttcatccgcgatatcgcagtcggcgtcacaggttgcccgtgagacaaaggtacgccggaaactggtaaaggaaagggccaggctgaaaagggccacggtcaaaaatccgcaggccagaatcaaagttaaccggggggatttgcccgtaatcaagctgggtaatgcgcgggttgtcctttcgcgccgcaggcgtcgtaaaaaggggcagcgttcatccctgaaaggtggcggcagcgtgcttgtggtgggtaaccgtcgtattcccggcgcgtttattcagcaactgaaaaatggccggtggcatgtcatgcagcgtgtggctgggaaaaaccgttaccccattgatgtggtgaaaatcccgatggcggtgccgctgaccacggcgtttaaacaaaatattgagcggatacggcgtgaacgtcttccgaaagagctgggctatgcgctgcagcatcaactgaggatggtaataaagcgatgaaacatactgaactccgtgcagccgtactggatgcactggagaagcatgacaccggggcgacgttttttgatggtcgccccgctgtttttgatgaggcggattttccggcagttgccgtttatctcaccggcgctgaatacacgggcgaagagctggacagcgatacctggcaggcggagctgcatatcgaagttttcctgcctgctcaggtgccggattcagagctggatgcgtggatggagtcccggatttatccggtgatgagcgatatcccggcactgtcagatttgatcaccagtatggtggccagcggctatgactaccggcgcgacgatgatgcgggcttgtggagttcagccgatctgacttatgtcattacctatgaaatgtgaggacgctatgcctgtaccaaatcctacaatgccggtgaaaggtgccgggaccaccctgtgggtttataaggggagcggtgacccttacgcgaatccgctttcagacgttgactggtcgcgtctggcaaaagttaaagacctgacgcccggcgaactgaccgctgagtcctatgacgacagctatctcgatgatgaagatgcagactggactgcgaccgggcaggggcagaaatctgccggagataccagcttcacgctggcgtggatgcccggagagcaggggcagcaggcgctgctggcgtggtttaatgaaggcgatacccgtgcctataaaatccgcttcccgaacggcacggtcgatgtgttccgtggctgggtcagcagtatcggtaaggcggtgacggcgaaggaagtgatcacccgcacggtgaaagtcaccaatgtgggacgtccgtcgatggcagaagatcgcagcacggtaacagcggcaaccggcatgaccgtgacgcctgccagcacctcggtggtgaaagggcagagcaccacgctgaccgtggccttccagccggagggcgtaaccgacaagagctttcgtgcggtgtctgcggataaaacaaaagccaccgtgtcggtcagtggtatgaccatcaccgtgaacggcgttgctgcaggcaaggtcaacattccggttgtatccggtaatggtgagtttgctgcggttgcagaaattaccgtcaccgccagttaatccggagagtcagcgatgttcctgaaaaccgaatcatttgaacataacggtgtgaccgtcacgctttctgaactgtcagccctgcagcgcattgagcatctcgccctgatgaaacggcaggcagaacaggcggagtcagacagcaaccggaagtttactgtggaagacgccatcagaaccggcgcgtttctggtggcgatgtccctgtggcataaccatccgcagaagacgcagatgccgtccatgaatgaagccgttaaacagattgagcaggaagtgcttaccacctggcccacggaggcaatttctcatgctgaaaacgtggtgtaccggctgtctggtatgtatgagtttgtggtgaataatgcccctgaacagacagaggacgccgggcccaaataaacttataaattgtgagagaaattaatgaatgtctaagttaatgcagaaacggagagacatactatattcatgaactaaaagacttaatattgtgaaggtatactttctttccacataaatttgtagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagccattttcatatttttcttgaacattaagtacaagtctataattaaaaattttttaaatgtagtctggaacattgccagaaacagaagtacaacagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaaagagattgtagaaagttgatacaatttttttttctttaaatatatgcatccgctggatttcagtctgctgaaccggcgcgaggctgacgaagagcctgaagatgatgtgctgatgcagaaagcggcagggcttgccggaggtgtccgctttggcccggacgggaatgaagttatccccgcttccccggatgtggcggacatgacggaggatgacgtaatgctgatgacagtatcagaagggatcgcaggaggagtccggtatggctgaaccggtaggcgatctggtcgttgatttgagtctggatgcggccagatttgacgagcagatggccagagtcaggcgtcatttttctggtacggaaagtgatgcgaaaaaaacagcggcagtcgttgaacagtcgctgagccgacaggcgctggctgcacagaaagcggggatttccgtcgggcagtataaagccgccatgcgtatgctgcctgcacagttcaccgacgtggccacgcagcttgcaggcgggcaaagtccgtggctgatcctgctgcaacagggggggcaggtgaaggactccttcggcgggatgatccccatgttcagggggcttgccggtgcgatcaccctgccgatggtgggggccacctcgctggcggtggcgaccggtgcgctggcgtatgcctggtatcagggcaactcaaccctgtccgatttcaacaaaacgctggtcctttccggcaatcaggcgggactgacggcagatcgtatgctggtcctgtccagagccgggcaggcggcagggctgacgtttaaccagaccagcgagtcactcagcgcactggttaaggcgggggtaagcggtgaggctcagattgcgtccatcagccagagtgtggcgcgtttctcctctgcatccggcgtggaggtggacaaggtcgctgaagccttcgggaagctgaccacagacccgacgtcggggctgacggcgatggctcgccagttccataacgtgtcggcggagcagattgcgtatgttgctcagttgcagcgttccggcgatgaagccggggcattgcaggcggcgaacgaggccgcaacgaaagggtttgatgaccagacccgccgcctgaaagagaacatgggcacgctggagacctgggcagacaggactgcgcgggcattcaaatccatgtgggatgcggtgctggatattggtcgtcctgataccgcgcaggagatgctgattaaggcagaggctgcgtataagaaagcagacgacatctggaatctgcgcaaggatgattattttgttaacgatgaagcgcgggcgcgttactgggatgatcgtgaaaaggcccgtcttgcgcttgaagccgcccgaaagaaggctgagcagcagactcaacaggacaaaaatgcgcagcagcagagcgataccgaagcgtcacggctgaaatataccgaagaggcgcagaaggcttacgaacggctgcagacgccgctggagaaatataccgcccgtcaggaagaactgaacaaggcactgaaagacgggaaaatcctgcaggcggattacaacacgctgatggcggcggcgaaaaaggattatgaagcgacgctgaaaaagccgaaacagtccagcgtgaaggtgtctgcgggcgatcgtcaggaagacagtgctcatgctgccctgctgacgcttcaggcagaactccggacgctggagaagcatgccggagcaaatgagaaaatcagccagcagcgccgggatttgtggaaggcggagagtcagttcgcggtactggaggaggcggcgcaacgtcgccagctgtctgcacaggagaaatccctgctggcgcataaagatgagacgctggagtacaaacgccagctggctgcacttggcgacaaggttacgtatcaggagcgcctgaacgcgctggcgcagcaggcggataaattcgcacagcagcaacgggcaaaacgggccgccattgatgcgaaaagccgggggctgactgaccggcaggcagaacgggaagccacggaacagcgcctgaaggaacagtatggcgataatccgctggcgctgaataacgtcatgtcagagcagaaaaagacctgggcggctgaagaccagcttcgcgggaactggatggcaggcctgaagtccggctggagtgagtgggaagagagcgccacggacagtatgtcgcaggtaaaaagtgcagccacgcagacctttgatggtattgcacagaatatggcggcgatgctgaccggcagtgagcagaactggcgcagcttcacccgttccgtgctgtccatgatgacagaaattctgcttaagcaggcaatggtggggattgtcgggagtatcggcagcgccattggcggggctgttggtggcggcgcatccgcgtcaggcggtacagccattcaggccgctgcggcgaaattccattttgcaaccggaggatttacgggaaccggcggcaaatatgagccagcggggattgttcaccgtggtgagtttgtcttcacgaaggaggcaaccagccggattggcgtggggaatctttaccggctgatgcgcggctatgccaccggcggttatgtcggtacaccgggcagcatggcagacagccggtcgcaggcgtccgggacgtttgagcagaataaccatgtggtgattaacaacgacggcacgaacgggcagataggtccggctgctctgaaggcggtgtatgacatggcccgcaagggtgcccgtgatgaaattcagacacagatgcgtgatggtggcctgttctccggaggtggacgatgaagaccttccgctggaaagtgaaacccggtatggatgtggcttcggtcccttctgtaagaaaggtgcgctttggtgatggctattctcagcgagcgcctgccgggctgaatgccaacctgaaaacgtacagcgtgacgctttctgtcccccgtgaggaggccacggtactggagtcgtttctggaagagcacgggggctggaaatcctttctgtggacgccgccttatgagtggcggcagataaaggtgacctgcgcaaaatggtcgtcgcgggtcagtatgctgcgtgttgagttcagcgcagagtttgaacaggtggtgaactgatgcaggatatccggcaggaaacactgaatgaatgcacccgtgcggagcagtcggccagcgtggtgctctgggaaatcgacctgacagaggtcggtggagaacgttattttttctgtaatgagcagaacgaaaaaggtgagccggtcacctggcaggggcgacagtatcagccgtatcccattcaggggagcggttttgaactgaatggcaaaggcaccagtacgcgccccacgctgacggtttctaacctgtacggtatggtcaccgggatggcggaagatatgcagagtctggtcggcggaacggtggtccggcgtaaggtttacgcccgttttctggatgcggtgaacttcgtcaacggaaacagttacgccgatccggagcaggaggtgatcagccgctggcgcattgagcagtgcagcgaactgagcgcggtgagtgcctcctttgtactgtccacgccgacggaaacggatggcgctgtttttccgggacgtatcatgctggccaacacctgcacctggacctatcgcggtgacgagtgcggttatagcggtccggctgtcgcggatgaatatgaccagccaacgtccgatatcacgaaggataaatgcagcaaatgcctgagcggttgtaagttccgcaataacgtcggcaactttggcggcttcctttccattaacaaactttcgcagtaaatcccatgacacagacagaatcagcgattctggcgcacgcccggcgatgtgcgccagcggagtcgtgcggcttcgtggtaagcacgccggagggggaaagatatttcccctgcgtgaatatctccggtgagccggaggctatttccgtatgtcgccggaagactggctgcaggcagaaatgcagggtgagattgtggcgctggtccacagccaccccggtggtctgccctggctgagtgaggccgaccggcggctgcaggtgcagagtgatttgccgtggtggctggtctgccgggggacgattcataagttccgctgtgtgccgcatctcaccgggcggcgctttgagcacggtgtgacggactgttacacactgttccgggatgcttatcatctggcggggattgagatgccggactttcatcgtgaggatgactggtggcgtaacggccagaatctctatctggataatctggaggcgacggggctgtatcaggtgccgttgtcagcggcacagccgggcgatgtgctgctgtgctgttttggttcatcagtgccgaatcacgccgcaatttactgcggcgacggcgagctgctgcaccatattcctgaacaactgagcaaacgagagaggtacaccgacaaatggcagcgacgcacacactccctctggcgtcaccgggcatggcgcgcatctgcctttacggggatttacaacgatttggtcgccgcatcgaccttcgtgtgaaaacgggggctgaagccatccgggcactggccacacagctcccggcgtttcgtcagaaactgagcgacggctggtatcaggtacggattgccgggcgggacgtcagcacgtccgggttaacggcgcagttacatgagactctgcctgatggcgctgtaattcatattgttcccagagtcgccggggccaagtcaggtggcgtattccagattgtcctgggggctgccgccattgccggatcattctttaccgccggagccacccttgcagcatggggggcagccattggggccggtggtatgaccggcatcctgttttctctcggtgccagtatggtgctcggtggtgtggcgcagatgctggcaccgaaagccagaactccccgtatacagacaacggataacggtaagcagaacacctatttctcctcactggataacatggttgcccagggcaatgttctgcctgttctgtacggggaaatgcgcgtggggtcacgcgtggtttctcaggagatcagcacggcagacgaaggggacggtggtcaggttgtggtgattggtcgctgatgcaaaatgttttatgtgaaaccgcctgcgggcggttttgtcatttatggagcgtgaggaatgggtaaaggaagcagtaaggggcataccccgcgcgaagcgaaggacaacctgaagtccacgcagttgctgagtgtgatcgcgatgccatcagcgaagggccgattgaaggtccggtggatggcttaaaaagcgtgctgctgaacagtacgccggtgctggacactgaggggaataccaacatatccggtgtcacggtggtgttccgggctggtgagcaggagcagactccgccggagggatttgaatcctccggctccgagacggtgctgggtacggaagtgaaatatgacacgccgatcacccgcaccattacgtctgcaaacatcgaccgtctgcgctttaccttcggtgtacaggcactggtggaaaccacctcaaagggtgacaggaatccgtcggaagtccgcctgctggttcagatacaacgtaacggtggctgggtgacggaaaaagacatcaccattaagggcaaaaccacctcgcagtatctggcctcggtggtgatgggtaacctgccgccgcgcccgtttaatatccggatgcgcaggatgacgccggacagcaccacagaccagctgcagaacaaaacgctctggtcgtcatacactgaaatcatcgcgatgtgaaacagtgctacccgaacacggcactggtcggcgtgcaggtggactcggagcagttcggcagccagcaggtgagccgtaattatcatctgcgcgggcgtattctgcaggtgccgtcgaactataacccgcagacgcggcaatacagcggtatctgggacggaacgtttaaaccggcatacagcaacaacatggcctggtgtctgtgggatatgctgacccatccgcgctacggcatggggaaacgtcttggtgcggcggatgtggataaatgggcgctgtatgtcatcggccagtactgcgaccagtcagtgccggacggctttggcggcacggagccgcgcatcacctgtaatgcgtacctgaccacacagcgtaaggcgtgggatgtgctcagcgatttctgctcggcgatgcgctgtatgccggtatggaacgggcagacgctgacgttcgtgcaggaccgaccgtcggataagacgtggacctataaccgcagtaatgtggtgatgccggatgatggcgcgccgttccgctacagcttcagcgccctgaaggaccgccataatgccgttgaggtgaactggattgacccgaacaacggctgggagacggcgacagagcttgttgaagatacgcaggccattgcccgttacggtcgtaatgttacgaagatggatgcctttggctgtaccagccgggggcaggcacaccgcgccgggctgtggctgattaaaacagaactgctggaaacgcagaccgtggatttcagcgtcggcgcagaagggcttcgccatgtaccgggcgatgttattgaaatctgcgatgatgactatgccggtatcagcaccggtggtcgtgtgctggcggtgaacagccagacccggacgctgacgctcgaccgtgaaatcacgctgccatcctccggtaccgcgctgataagcctggttgacggaagtggcaatccggtcagcgtggaggttcagtccgtcaccgacggcgtgaaggtaaaagtgagccgtgttcctgacggtgttgctgaatacagcgtatgggagctgaagctgccgacgctgcgccagcgactgttccgctgcgtgagtatccgtgagaacgacgacggcacgtatgccatcaccgccgtgcagcatgtgccggaaaaagaggccatcgtggataacggggcgcactttgacggcgaacagagtggcacggtgaatggtgtcacgccgccagcggtgcagcacctgaccgcagaagtcactgcagacagcggggaatatcaggtgctggcgcgatgggacacaccgaaggtggtgaagggcgtgagtttcctgctccgtctgaccgtaacagcggacgacggcagtgagcggctggtcagcacggcccggacgacggaaaccacataccgcttcacgcaactggcgctggggaactacaggctgacagtccgggcggtaaatgcgtgggggcagcagggcgatccggcgtcggtatcgttccggattgccgcaccggcagcaccgtcgaggattgagctgacgccgggctattttcagataaccgccacgccgcatcttgccgtttatgacccgacggtacagtttgagttctggttctcggaaaagcagattgcggatatcagacaggttgaaaccagcacgcgttatcttggtacggcgctgtactggatagccgccagtatcaatatcaaaccgggccatgattattacttttatatccgcagtgtgaacaccgttggcaaatcggcattcgtggaggccgtcggtcgggcgagcgatgatgcggaaggttacctggattttttcaaaggcaagataaccgaatcccatctcggcaaggagctgctggaaaaagtcgagctgacggaggataacgccagcagactggaggagttttcgaaagagtggaaggatgccagtgataagtggaatgccatgtgggctgtcaaaattgagcagaccaaagacggcaaacattatgtcgcgggtattggcctcagcatggaggacacggaggaaggcaaactgagccagtttctggttgccgccaatcgtatcgcatttattgacccggcaaacgggaatgaaacgccgatgtttgtggcgcagggcaaccagatattcatgaacgacgtgttcctgaagcgcctgacggcccccaccattaccagcggcggcaatcctccggccttttccctgacaccggacggaaagctgaccgctaaaaatgcggatatcagtggcagtgtgaatgcgaactccgggacgctcagtaatgtgacgatagctgaaaactgtacgataaacggtacgctgagggcggaaaaaatcgtcggggacattgtaaaggcggcgagcgcggcttttccgcgccagcgtgaaagcagtgtggactggccgtcaggtacccgtactgtcaccgtgaccgatgaccatccttttgatcgccagatagtggtgcttccgctgacgtttcgcggaagtaagcgtactgtcagcggcaggacaacgtattcgatgtgttatctgaaagtactgatgaacggtgcggtgatttatgatggcgcggcgaacgaggcggtacaggtgttctcccgtattgttgacatgccagcgggtcggggaaacgtgatcctgacgttcacgcttacgtccacacggcattcggcagatattccgccgtatacgtttgccagcgatgtgcaggttatggtgattaagaaacaggcgctgggcatcagcgtggtctgagtgtgttacagaggttcgtccgggaacgggcgttttattataaaacagtgagaggtgaacgatgcgtaatgtgtgtattgccgttgctgtctttgccgcacttgcggtgacagtcactccggcccgtgcggaaggtggacatggtacgtttacggtgggctattttcaagtgaaaccgggtacattgccgtcgttgtcgggcggggataccggtgtgagtcatctgaaagggattaacgtgaagtaccgttatgagctgacggacagtgtgggggtgatggcttccctggggttcgccgcgtcgaaaaagagcagcacagtgatgaccggggaggatacgtttcactatgagagcctgcgtggacgttatgtgagcgtgatggccggaccggttttacaaatcagtaagcaggtcagtgcgtacgccatggccggagtggctcacagtcggtggtccggcagtacaatggattaccgtaagacggaaatcactcccgggtatatgaaagagacgaccactgccagggacgaaagtgcaatgcggcatacctcagtggcgtggagtgcaggtatacagattaatccggcagcgtccgtcgttgttgatattgcttatgaaggctccggcagtggcgactggcgtactgacggattcatcgttggggtcggttataaattctgattagccaggtaacacagtgttatgacagcccgccggaaccggtgggcttttttgtggggtgaatatggcagtaaagatttcaggagtcctgaaagacggcacaggaaaaccggtacagaactgcaccattcagctgaaagccagacgtaacagcaccacggtggtggtgaacacggtgggctcagagaatccggatgaagccgggcgttacagcatggatgtggagtacggtcagtacagtgtcatcctgcaggttgacggttttccaccatcgcacgccgggaccatcaccgtgtatgaagattcacaaccggggacgctgaatgattttctctgtgccatgacggaggatgatgcccggccggaggtgctgcgtcgtcttgaactgatggtggaagaggtggcgcgtaacgcgtccgtggtggcacagagtacggcagacgcgaagaaatcagccggcgatgccagtgcatcagctgctcaggtcgcggcccttgtgactgatgcaactgactcagcacgcgccgccagcacgtccgccggacaggctgcatcgtcagctcaggaagcgtcctccggcgcagaagcggcatcagcaaaggccactgaagcggaaaaaagtgccgcagccgcagagtcctcaaaaaacgcggcggccaccagtgccggtgcggcgaaaacgtcagaaacgaatgctgcagcgtcacaacaatcagccgccacgtctgcctccaccgcggccacgaaagcgtcagaggccgccacttcagcacgagatgcggtggcctcaaaagaggcagcaaaatcatcagaaacgaacgcatcatcaagtgccggtcgtgcagcttcctcggcaacggcggcagaaaattctgccagggcggcaaaaacgtccgagacgaatgccaggtcatctgaaacagcagcggaacggagcgcctctgccgcggcagacgcaaaaacagcggcggcggggagtgcgtcaacggcatccacgaaggcgacagaggctgcgggaagtgcggtatcagcatcgcagagcaaaagtgcggcagaagcggcggcaatacgtgcaaaaaattcggcaaaacgtgcagaagatatagcttcagctgtcgcgcttgaggatgcggacacaacgagaaaggggatagtgcagctcagcagtgcaaccaacagcacgtctgaaacgcttgctgcaacgccaaaggcggttaaggtggtaatggatgaaacgaacagaaaagcccactggacagtccggcactgaccggaacgccaacagcaccaaccgcgctcaggggaacaaacaatacccagattgcgaacaccgcttttgtactggccgcgattgcagatgttatcgacgcgtcacctgacgcactgaatacgctgaatgaactggccgcagcgctcgggaatgatccagattttgctaccaccatgactaacgcgcttgcgggtaaacaaccgaagaatgcgacactgacggcgctggcagggctttccacggcgaaaaataaattaccgtattttgcggaaaatgatgccgccagcctgactgaactgactcaggttggcagggatattctggcaaaaaattccgttgcagatgttcttgaataccttggggccggtgagaattcggcctttccggcaggtgcgccgatcccgtggccatcagatatcgttccgtctggctacgtcctgatgcaggggcaggcgtttgacaaatcagcctacccaaaacttgctgtcgcgtatccatcgggtgtgcttcctgatatgcgaggctggacaatcaaggggaaacccgccagcggtcgtgctgtattgtctcaggaacaggatggaattaagtcgcacacccacagtgccagtgcatccggtacggatttggggacgaaaaccacatcgtcgtttgattacgggacgaaaacaacaggcagtttcgattacggcaccaaatcgacgaataacacgggggctcatgctcacagtctgagcggttcaacaggggccgcgggtgctcatgcccacacaagtggtttaaggatgaacagttctggctggagtcagtatggaacagcaaccattacaggaagtttatccacagttaaaggaaccagcacacagggtattgcttatttatcgaaaacggacagtcagggcagccacagtcactcattgtccggtacagccgtgagtgccggtgcacatgcgcatacagttggtattggtgcgcaccagcatccggttgttatcggtgctcatgcccattctttcagtattggttcacacggacacaccatcaccgttaacgctgcgggtaacgcggaaaacaccgtcaaaaacattgcatttaactatattgtgaggcttgcataatggcattcagaatgagtgaacaaccacggaccataaaaatttataatctgctggccggaactaatgaatttattggtgaaggtgacgcatatattccgcctcataccggtctgcctgcaaacagtaccgatattgcaccgccagatattccggctggctttgtggctgttttcaacagtgatgaggcatcgtggcatctcgttgaagaccatcggggtaaaaccgtctatgacgtggcttccggcgacgcgttatttatttctgaactcggtccgttaccggaaaattttacctggttatcgccgggaggggaatatcagaagtggaacggcacagcctgggtgaaggatacggaagcagaaaaactgttccggatccgggaggcggaagaaacaaaaaaaagcctgatgcaggtagccagtgagcatattgcgccgcttcaggatgctgcagatctggaaattgcaacgaaggaagaaacctcgttgctggaagcctggaagaagtatcgggtgttgctgaaccgtgttgatacatcaactgcacctgatattgagtggcctgctgtccctgttatggagtaatcgttttgtgatatgccgcagaaacgttgtatgaaataacgttctgcggttagttagtatattgtaaagctgagtattggtttatttggcgattattatcttcaggagaataatggaagttctatgactcaattgttcatagtgtttacatcaccgccaattgcttttaagactgaacgcatgaaatatggtttttcgtcatgttttgagtctgctgttgatatttctaaagtcggttttttttcttcgttttctctaactattttccatgaaatacatttttgattattatttgaatcaattccaattacctgaagtctttcatctataattggcattgtatgtattggtttattggagtagatgcttgcttttctgagccatagctctgatatccaaatgaagccataggcatttgttattttggctctgtcagctgcataacgccaaaaaatatatttatctgcttgatcttcaaatgttgtattgattaaatcaattggatggaattgtttatcataaaaaattaatgtttgaatgtgataaccgtcctttaaaaaagtcgtttctgcaagctcctaggtttttggcgcgccctacgtcacccgccccgttcccacgccccgcgccacgtcacaaactccaccccctcattatcatattggcttcaatccaaaataaggtatattattgatgatgttacatcg

SEQ ID NO: 74 - DNA, 26597 bp, Artificial Sequence
  pGLAd3
ttaattaagcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcttaattaaatcgatttaagggtgggaaagaatatataaggtgggggtcttatgtagtttgagctcggctttggttatttgatggattgacctagttctcaggcagccacctgtgtggactaaatacacccatttcttctccattctgaaggatgtgctgaaagccgccttggacagtgagaaaagcaaacatggaagcctgctgtaggatcaccttacaccatcagatacccacttcccacccccggcttcctgaactttctgtaattactcatgccagtgactagggctcatgtgggccatcccaggaaggagactagtggctgccattctgtcctggtctttgtgtctcccgaggaggagtgtaggtggtgaggtcacaggctaagccaggccatttaaggaaagctgcctcagctccagcccagccctctctgccaggtggatatggaaatcccggtttactctctggggccttttattggctgaaatgctcaccttggtatctgttacacattcccttccaagagcagagctagataagccaggtgtggagacaatcagctcttgaaatctcccagaagctgccaaacccctaaaatcagcaagagacacccctcatagtccagactcagctgttttattctgcctctccctgatctcttttcagggcataaagttttccaaggagaagaatgaaatcagtatagcaggtcattgagaaagtatagaaaggctcaggttgaacttcagtgtctgctaccaatgtgtcataaataagtcattttactactgtgagactcagtttacccatttgttgagtatgaaaaaaaatcatatatatttgttatgctatatgtatcaatgcatcgagctagactctggggctaaagcaatgagtaaaacgtatttggctgaacataaatatgggaggggggtggaatgcatgaatgtaaagaatgctggcataagtaggaccagtgagttgggtgtggcagtgcaccttggagacagagccaggcggacctctgagttcaaggacagtcatcagggctccacagagaaatcctgtccaaaaaaaaaaaaaaaaatcagtggggaaaggcacctgccatcgaggctgaagacctgaatttggtccacaggatccacgtggtaggaaagaaagaacttccaagggttattctctaacctccatacttatatgtgagtgtacacttaagtaatgtttgtttgtttgttttgttcctgttatgttttgtttgtttttaaagccaagtctggtggttcaggcatttaatcatagcacccagaaggcagagttcaagggcagcctggtctacataggaaattgtaggcctatacagtgaaaccctgtttcaaaggaagattaaaggaaaaaagtcttgcactcttccacaccctgaccaaagtcctatagtgaaactctggtcccctccctcatcactgctattgccaaactcagtctccctctatgacccacatcatgatagccagggctagaccgtttgtaaagataaatcagccttgtctggataaataactccaggaaggagaaactgccggctgtaaagaaccttagcagtcgtattccccaaaaccacctatataagcctcaagtgcccctccacctttcacagacacccctcccctacgggagagtatctgtcctgcactgtttgaaataaacagtcctgtctgttgaggtcagtcttcagtctttttttctgcttccagtctcttaagttgcttcagaaatttaccaagaatcactaaataaagccagctgtgatggtccacatctgtattccagtgcttcgaaggctgaggcagagggtctcaagtgtgaggctagcctgggctgcctgtggaaccctgcctttttttggggagcaggcttttaaaaaatatttatcacggtgaaatataaggcaggaggattgttagaagttcaataaggacaatctgattctgtagtggggttcaggctagctagggctacatactaaacaaggtctcaaccttgttttagtaagaggaggaggaggaaacagaaggaaaaaaaagtaatttcttaggcacccagtaggcctgcaagtcttgttttgaacaaatgaatggatgcatgaatcaatctttgctacctttcaggctctattctgtgtccccagagcacccccggcccaggaaagtgagcagggcacggtaggtctgctggaatctctgattgtacttgtgagggctctgcctagcagtctccctggaagctgagcctggccttagcctcttccttctctctcagagcttctgaagccacagaagggacgcatcaaactcggacctagaaatgaacgattagtcttttttagtgattttttttcttccgtctttattttttgacactcactgggtagccttggctggcccagagctccctaggttggtcagagatctgcctgcctcctgcctgggtgttgatatacagggattcaccactctatgctcttacgagctggcttcgaactcatcaggcttagaatgtcgggtcgtctcacttatacctcctaagtgttggaataaccagtgtgcaccacccaagagcccctagctgggactcttgccaggcctgtcttctctaagccttagtatccccatctccaaaatgggtataaccatatgcatacgtatcctaagggcatataccaaaagttcagtgaatacagtagagtatgtaggaccccctgcgggctagcagtagaagcctttccctttccattatacccttggttctaaagttggttgaaagttcccctaagctctctcccgcccagcaaaccagtcaaaccagtttcctcattccccgggcttctgtactaagcaaacactgctttgcaaacggttgaccaggagcttgcctttgttagagaaggaagtccaatggtgttttcttccttgcaatgccgggagtgggtttgctgtgccttagaaagggttcaaactgggctggaagtagggccccagagtagctgggggttggggattcaggatggcctcaagtttgttgttccccaaaccctcactttttctagaactatttcataatacacatgcttaaggatacccatttgttgacccattgcaaaatgctctctgacccagcttctgagactggttcaccccccatggataagaattgagtcatctggtgccattcacaaatacagattaacctccctcccccaatttactgaacctcaaccaaaaaaagatccgcctgcctctgcctcccaagtgctaggattttaaagggccacggtaccacacggatggacacaacattcattttcctgacagaccaggagtcaggtcacaggtgtttgactagtaaacacccggtgccaaccagggtgtgggcatttggctatctaccctgaggcccaagatccaagagcacccgtggtccatggtgctaggggacccggaaatgtagccagtgcatcagagatccagcctctgtcctcagggaagttcagaaacatctctgcttactgagacttctgcagggtatttgggaagaggggccagcacagcaggcatgatcagttttacatgctgggcctttgtaagtaagaggatgtgtccaggcacagtaaaggaaagtctcccccaggaccttgggcctttttatccactggtgaggctttttttctctttctgttcccccctccccacctcccgggctttccagacagagtttccctgtgtattcattcctggctgtcctggaacttgctaggtagaccgggctggcctcgaactcagtatgaacaaatgtttacttatggctggcactgagacttcctcttcaccctgactcctcacccagagcttcccctgaacagaattctcttggttagccttctcaagatcttctgtggaaaaggcagagatagtcatggggtgtggctctgtggtcaggctaaacctaatgagtgtgaggccctgactttgattcctagcattgaggaaaagcagaaacaggaatcttacccaattttcctctgcctgttaccagactcaaggctgtcccgtggagcccattgcccttcctgagaaacccacttcctttcctgaggctaggaaagacatccctcatctctgtgcctcaatctcattgatttttgtggtgttaaaattcagttatttgtctttgagagggctcccctcccccctttgcctttcacatcctttcccttctttattgctatgcagaggaccctaactcctaggcaggccctctacccttgagatacagctccatcctcttgctttttaaaaacttataattcgagacagggtctcactgctacccaggctggttttgagcttactctgcagcctcctgcatcagcccaccgccttgtacgtgagatggtgggcctgagactcagcctcttgaccttcacatacatctgatacgtgctatctgaacaggcctgtcttactcagttttctgggtcctgttggggggtgaggtggggaggggagatcttcgttcaggcatggagaggtgggaaggaaaccagtccacacctctaacaagtgagttccaactaggacttgcttagggcacataaggcagacagacgtgggagatgtgagcggcggggaagcgatgatgcttagatgctaccacgaggacctgatgtggtgtgcctgaagatgaacctgttccccatgagccagctcataaatggggaccagcaccggcccctgtgggataattggcctcgtcctagataataattacttagctttgtaattaggagagcttgtaagaagccgaaagtgtttcggatttattttacctggacaagttcaagactaaactaacacctgttcctgtggtttggcatgcagagccatctgtttccaaaggaggtcctgcagcagctttttttggggttctcctccctgttccttcccttctctccccacatcgccttttctccgtccagtggtcagtgtgtgtgtgtgtgtgtgtgtgtgtgtgtgtgttaatgagacagggtctcattatgtagccttgggcctggaactcactatgtagatgtggctggcttcaaactcagagaggcatatctatccacttgcctttgcctctgcctctcaagcggccgcgtgctgagactaaagggatgtgctactactcctggccagagtgataagtactcatcctgtttaaagttgcctgggattccctgtttgacactgaataaaaatcctgcccatgacttccaaggctctgggtccctccttcccccgcagcccatggcttgggccttcattttaaccccactccccacatgtgcttcgttcagatgctttgcttcctgattgtactcagaataccatggcagctcgggctgaggctggttttgaactcttaatactcctgtccgggagtagtagcatctcaggtgtgtccaatcacactctatgtattccttgtttgtgatgtctgtatcactcacccgtgcccttcaaagctttgctaagatgttgcctgggcaatgaggaccttctctttttgctttatttttctgtagccccccacctccccgttttttgggtggttttggtagggatgagacattaagatgttgcctgggcaatgaggaccttctctttttgctttatttttctgtagccccccacctccccgttttttgggtggttttggtagggatgagacaacagggccttatgcaggttatgcaagtaccctattgctgatccacatcccagcctttctggtattttccaccattgaacctgtcatgtggtttgtgtatttcctcagttcatagtcttcttccataaaaacataaatgtggccggacgtggtggcgcatgcctttaatcccagcactcgggaggcagaggcaggtggatttctgagttcgaggccagcctggtctacaaagtgagttccaggacagccagggctatagagaaaccctgtctcaaaaccaaaaaaaaaaaaaaaaaaaaaaaaaaaccaaaaccataaatgtgaggaggtcagggtgttctgtgtttgttggttcttgagctgtacttcaggccgtaacaagttcttagacctcatctgttgaatgagtgaacggaggagtgagaggtccagaaggtaaatatggcgctgtacagttctttttagaagctgtgctgtgggttttgttttttctaagataaaatggggtccagtgacatggctcagagtaaatgtaaatgttgtaccttttggcatcctgggttttatttttccagaacccatttatcctgtgacttccagatgcgcaccataccccctgtaaacccccccccccccaacacacacacacaccaaactagcaaacagatacagggctgtcagcgtgtgagcacatgaggaagggatgctatgttctcaggccctgtgtttcttcttggcaccccctggtttctctgagccctgcacagctacagtgtaaccttacctaattgtcttgttacaaagaagtttgctgcccagagtctaaaggtcagatgagtcacttgggcctcacacttctgggtgtatggaagcaccagatggaagtctcaaggtacacccgacaccggctgcctttccgtcgtcaggcagcacattcctgaagcaggctctctcagctgtgccctctcaaccttcatcctgcagcccattaattgttcaaggccttctagttcttggcaggtctgagcatcttggaaggagctagaagagagatgtagaaagctcaaagacaaaaatgcaaagctacccccatcagataccagctcttgggtctattgaccagccaatatccattgatagtggagttctacatccatatggaagatacaccattctctttcatggacaggagtgccaaatcagaatacaggacattacatctttttgagacaaggtcttaccatgtagcctttgtagaccagctatgtagaccatgctgccctggaacccacagagatttgctagcttctgcctcccaagtgctaagattagtgacacacacctccacttacacagtacacacccaggaaaaaagtaaaacataaataaaaagcaaaaaattgttgtgaaatagctcagtattagagtccttgtctaagatatatgggctccaggcttgcccatccatctattttctgagactggatttttctgtgtagccctggctgtcctgggtcttactttgtagaccaggctgaccttgaactcagagatccacctgcctctgcctctggagctctggggttaaaggtagaccccaccagccatgggcttaagcccagtatgggtttaaaaacatggaaccgattggactttgcataaagttagattggatcctgtgggtgctgacctcccatgctttcaagcacttattggtagcattcactgaacaagcctgagggcgggaacagccctggcatctttcaaagggtatccggttcaaaccagaatcgaatgctcttttcctagccaccccacctcttggcttgttgcctaaggaaaatggctctagcctggttctccagtgttctcatggcagtctggagagattgtccatttgtatcacaaccttttgggcgctgcccttggtgacccgctgctgatgtggacaccagcaagacttgagactgcagacctcctcccccacagatttctcacaatgcccgtgtgggagtgtttgaaaagggaacaagctgaccaacctgtttgtgggaaagtcgtggctttcctatcagaaaacgtcacactctgagtcaggagtgggacatacctgggtactttaacaggtggggcagggtcctgagtcatagagaggtctcacctgggaaggtttctctttcaaagagtgaatcaaccatgacatcacccatctggatctacaccctttcaagccgtctctctccttagcaaacaggaatctaaaagaggaataataaggtgaaagcaaactggaaataggacagaacagacaagggaaaaagttaaagagaaagcttaagagacgcagacacagacgtttacatgcaaacagggggtccataaaaacaaaaccggaagccgtaatatatgtacatggcctgtaagattagaaacaaatgtcctggcacaacattatgggacaacttcccaagctgctgttgagtttgttttgtgttggccggcatgggcccttaaactgagtggtgtgtttctctcttagagaatttttcatgaactagtttttcatttgcaagtggttagtagtttgagatagcttctaggttaggggtggggccgtgtgtccactttactcagctctaatgtcccccgtttggtgcagacccctgcagaccctatgcgtcctgccacagtctctgagttcatgcctgcattgttcaaattgtgtttccttgatgtcctccaatctcctctttgctgggctccctggggcccccaaggtgagggatttggcagaggtattcattccatttagggttgattgttccaaggtccctcactgtacgtgttatttgtcccttctccctggcttcctgctcaggagttctgcagacatggctggtgtttagtagcttcttggtgaagttcaggtagccagtttcatgaccacccagctctcaggcctgactaagcccgtggagaaagcccataaaataaagggggtgaacttctaatctacatctgaaaggagaacccctgtctacatctacctgttaccctagccatcggggagactgaggcagaaggatctagcctggaaatttagggtgagtctgggcagcattaggagactatttcacagtgaagaaggaaggaactcactcacacccttggtgcacctcacaggtctctgggctcagattggagtctttggttttgaaatttcctcatcatcggccgcctcagctggtttttcttctggagtctgttacgccttctcccctttccccccttctcttgctaatcctttcgcttgaacaataggagtccttaaaacctgggagggaagtaaatacctttcagatgagaccctgtcttaagagggggaaaaaaaaaacaaaaaaaccctagggaccgggtcaccaagtgtcaggttaatcagcagatgtcaagatgactcctgagtgcttgctctttcttcagctggtgaaattacagcggcaacatagaatgacagtcagcagggagggaggagagggcctgggtggccaaataaacttataaattgtgagagaaattaatgaatgtctaagttaatgcagaaacggagagacatactatattcatgaactaaaagacttaatattgtgaaggtatactttctttccacataaatttgtagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagccattttcatatttttcttgaacattaagtacaagtctataattaaaaattttttaaatgtagtctggaacattgccagaaacagaagtacaacagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaaagagattgtagaaagttgatacaatttttttttctttaaatatgtcgacgcactggctttacaggggccgtctgctttgtctgtagatatggtgataggagtctgatttagaaacttccttgttctcctcttcccgaatccttcctcctgaacaataggaatacctaagtgcacaagtgcaggtgcccaggaggccctgagacaaccccccctccccagctgcgagccttctccggtggtgctgggaatcgaactggagtcctctgcaagaacagtatgtgctcttaaccactgcaccatctcttcagccccctctagttctttagtttgtacataaattagcaagttcagggaatgcagaagaactggccaggggtcagagccagactggaaacatttttcaccaaacaatagctacagtgttcatggcgtgcctccattgaatactaaactagctgttgatgtgtgactttgatttttaatctttgcaccaactttagttactatatagataattccatttccaaatgatatatagtggaagcttgtaaaagctgcttactatcccagggtccccagccgagatctacatgataaagctactctcaacccagcctacattccccctccctgtgctcagccctacctggggttgttcactttcaagctcccaggaaggaacctgagcgctccctgcccccatgcagtccatttctatctctggtcctttgggtacttggagcctcaagctcctgactgctaatgaagcctgtaacagaagggcattacttactgcccgccttgtgctcccttgaaatctggtcacctggaggtggaagtgggcagtgcttccgtgcttccgtgcttccgctttctcccagctccttttaatcactattttttttttttcttttccaaattactacctaggagttttctagtaatcactttggtgggagtgggagggggagtggaggcagttaactctgtgagtgccaaggacagttgttggacggtggtagtgatggaggggagggtgttcacggtgagggaactctgtggagttacgtagctggcaacaatggggagagacggagccgcaccaccacagacgctttgcatgagcaactattttgttttctgtagatgttttccaaaccttaaactctgttaagtctgaaactgggaggggaacctctccaccaaaatctaaatatggatcagagataaggaaacttccatagtctccagctccctccctgactcagtggcctctgtgaccaactccacgtgactctagtggacaccttcctggtggtttggctttttttttttttccccatttcatcttttaaatggagttttccatgaataagaatataaaactgcgggggctggtgagatggatcagcaggtaagagcacagaggtcctgagttcaaatcccagcaaccacttggtggctcaccatcggtaacgagatctgactccctcttctagagtgtctgaagatagctacagtgtacttacatataataaataaacaaatcttaaaaaaaagaatataaaactgcttccttcccaacaggccttggatagttttgtctgtgggaacagcttcccataggaaaggtgcttttgctacttccgaccgtcacacactcctcctctgcaccccgtgagggcctcccaggagttttgtgttggtcacaagcctccccatgtcccaagggggtctggagagggatctgaggtggtggaaagtgctggggtgccaagttatcagacaatgggcacattgagaatgcaggtggggccaggcctgtgtgggctggagcagctgtcggagcctcagggggtgcagctcatgcgcagagggaagaggcagtctctctgctggctgagaggggagggccacagtaagcaaaggaggggggtgatccccccctcacaagattccagagcaaagggatgtttgaacgatgactcacatgacaggggcggggtctgtgagcaaacttcctgttggaagcttgttcttggttcctgactgtccttcccaggtcatagagactgtctgcaatagggctggggcctgttaggatgccatcgaggtgggaaatcctggagagcttggagtagccagcaggccctcttccctctcctgccccaggctagctggaaacccgcgaggaggaagggtgtgttgttccctatcccatggagaggcttgcatggtcagaccaggcctggtgtgacctctggggctgctctgtgactgccctgttctgtgactgcccttctccctcccagccctccccttccatttgcttctagattcgtctaggagcccatctcagcaaatggcagcatcctcgcttaccagacatcttgtttgtttgtttgtttgttcattcattctgggcctgaagatggagagctgtggtaggggagagctcagaggctgggggcagccttaagggcacagccaggacggagcctctgagcaaagggcagtgtgtgtagagtcaggtaggtggtgggcagcaggagacaggaggagcacagaggtaggtggcactgtacagaggccctggcaccattttgtgacaaggactctcagacccctctagcctattcccccacccccacccccattacaagcagtagggtcttaatagctaatggcttggacaccagcctctggctgctctggttcccagctggtcagagcccttgttctcctttgggttctgatccaggtggcacagggtgggggccctgattcacattcttgatagtgccgctaggttccctgctaccagctggcacattctgtcttcctttccagcaggtacttggctgggcttcctgcccagctgctagacctactgttaggtcagctcattggatgcttcctccaggaagccttctctaatctccattcccttggcttcttttcaagagcagaagttcactggcttctcctgtttccccagagtagcacctggtgtctcaagtactcgctaagcagttaggagaggtgtgaactgggcctcagggaccccagagacaggaggggaggtcattacaggaaagtcggattcttttcctggctttgttcttataaccatcgtgtgtgtgtgtgtgtgtgtgtgtgtgtgtgtgtgtgtgtgtgtgtgtgtgtacgtgtgtgtacgtgtgtgtgtgtgtacatgcgtgcgcgcacacgtgcacgcggctgatgccctctttctggccaccctttatttatttgtaatatttatttatttatttatttatttatttttggtttttttgagacagggtttctcgtatagccctggctatcctgaaacccactttatagaccaggctggccttgaactcagaaatctgcctgcctctgcctcccgagtgctgggattaaaggtgtgcgccaccactgcccggcttctgttcactcttaagcgtacccaacagcccatcctgcaccagcccagagagtctgcagagtcccctgtgaaaatgtgggcaacctgttcctcctggcatcaggtggggaggagaggcagattatgcagaatcaatcaatagcaatcaataggtccaatgcggaggagacaaaggattgagatgagacagggggcgtcccacgtccaattcctttattgctgcagtcctttctgcccctctttacaaacaggagactgaggtttagaaaggcaggctagggtagtgggtggaagggtgaggtgaacctacaggggagactaaggagccagatgaatggggaaagggaggcagcctcttaatccttttgtctgtagctccatctttggaaatgctgagtaattaatcaattgttaaagggctttctgccgaattctggggaaagcctttacttttaatgagctgctagtctgtctcttcagtgctgggaatgagtctagagccacctgtctcctaggcaaacactttactgctaaactactaagctctgctcccccacactccgggttagcctttacatctgtatctgtctgcctctgttgggatttcaggtgtgtgccaccccctgtgctcctcagtgtatttctacagggtggtggtacctaaggaaaaatccattcctagggactctgacgtaaatatttgggatgaggtctgtcaaggactgggcattttaacttcacttatttttatagccctgagggtggacatgggacctcttgtaggctaggtaggtttccttcccgatcgcccaagggcgaattccagcacactggcggccgttactagcggtagactaggatagcctcaaactcctaacggtctgcctgcctctacctcttggcctagggctgtggactgtctttttggtcctactataaggatcatctggaccagaacgcacgttctcaagcctacgctggggagtgcctcacctagaaagcttaaaagcgtagactctcgctggatgtggtggctcaggcctttgatcccagtactcaggaggcagaagcaggtagatctctgtgagttccaggacatccagggctacatagagagacattgtttgcccccaaagcacagaccctgacaggcctgtccatcttaaggaacggaggccagagacctgttggtagttgatggtttgatggtttatttagaaactctccctgatggttcttgacttggggagtgaggagaagccacttcaagttgtagacaaggtgtgtgtgtgtgtgtgtgtgtgtgtgcaagtgtgccaatgtgtaagtgtgtctgtatgccttgcacacacacagttgtttcaagtgtgctatgtagccaaggataacactgaactctgaattcccctttatctgtttatgggcacacatttcccccctgagggatataaattctccttgaagtgttgattcacccctgaagtagaatcaggtctaaagtggggatgaatctcagggtccttccaggtagaaatgtagggcaacaatgacagtacactgtaccctcctcctccctgtccttctccccgcttcttttttctttttgagctctgagtgactggaagtcatgtagattggctggtcccgatcttgtgcctttgcttcccgggtgctgagagtcgtgtgttctgaagaacagatttaagcaggcaagcaggctatgtaagatgtgtgacagaattccagagttgaagaggctggatggccaagggacctggctctgtccatccagccttcagtctgccttttcttctcgctgggagagtggcttggttaatccacaccagtgggtgactttacaaattaactcccgtgaccttggactgttccatccataaacacggctccctagtttatgagttgcccagcccagtgttaaaaatctacggtgaactaagccctgggagaagccagtgtgaaactcaaactgagcagagaccagatcaaacatggagataagacagctacagtttcacttttgggcagggccaagaatttcaggactttctttactacagatggggcgttattttgctttcctatgggtgtgtgtgtgtgtgtgctgatttagtttatgattcattgattttggggtttgatagcaggtctagggaacacataatgcgttacagatatttgattttaaatgtattcctggtactttttgggggggggttgtttattgttttgaggtttctcttttcccccggctgtcctagaactcatgctatagactagactgtcctcaaactcaagatttgcctgcttctgtttcctgagtattggaattaaaggcacgcaccaccactgccaagttgtttgcctggtattttagaaagggcaattagtcatgagcctgagggtttttcacatttatttgttttgcttgtaacaagttaaccagtgacaggtttactttagttctctctgtctctgtctttgtgtctctgtctcttatctttccttgacaggggggtctcatgtagcttcggttgaccccaaactcactattttagccaccagggcctttagcacctagtcttctgactgtaagtcctctgagtgctggggtcacaggtctaaccttctcattgaggaaactttggcctctatctctggtcatcaatactgatctggagatctctgagatactattactgtggaaggccatttgattctcttatgatggagcctgatgagccagaccagagaatggctcctattaggctcataaactggaatgcttactcactggggagtggcatcatttgaaaggattagaaggattaggggtgtggccttgttgggggaagtatgtcactggaggtgggctttgaggtttcaaaagcccatgtcaagaccagtgtgtgtgtgtgtgtgtgtgtatgtgtctgtctgtctgtctgtctgcctctttggatcaggttgtagctctcagctactgctccagcaccattcaagccaccatgatgattaatgggccaaacttttgaaactgttagacaagtccccagttaaacgcttttttggatatgagttccttaagtcacatgcctcttcagcaatagaacagtgatattggacaaccaggagcgtgctggaaaagtgagaaatttagaatttttcccagcagtgggaatttcctgtgtcctgatgccgatgccctcttttcaagactaaactgagaactttgctcctttgctgcttccctccttccatccttacctgctccagtgaagaatttgttgacatccgtatctcaggcacaagggggaaaacaaagggaaaataaagataagaggaggagggctgagtggcaaagtgagtgccatacagcttagaactctgcagaaagcagaccacaaatttgcctctgttcctgccaggcaaaataaagagggaaatctgatcagccctcctgctggacttcagtgtgctggccattagtggggtgcctggctcctccagagagttcttgcttcattgtagctcccaccactctaatgtgctagacttcccatttgcaattgtctttactagataaccaactaatgcttgggtggacaacattgcacaatagagggcagagatttcagtcatgtgtgtcaccacctacacagctccatgaggagcatttttgcatctttgcactcccccactccccgggtaagatctgtcttgtataatgcatagcataactatgtaatggtggggattttactaatgctattcactacccagcactctgccgcctcagggtcacgtgtttactattccctctgctgggcacctttgccttggcatatagcttataactccaggctctgttaaagcttcccccatagtggagtctaccacataataaaatgtacagttcctttttcatagcctttattataatccagcttaatactgtatgctatttgattgcttctccaacatcccccccctccaaatgtaggctccttgaaggtgcaacctgctttatttcattggttgctgatagaaaattttcaagtgatcctccccaccccgcccccctcacactcgctgcatgagatcaggtgctgggaattgaacccagcaccttacctgctacacatgctaggcaagtgctctaccatcgaggctccaggaatatggatctctctccttttctctccccactcctccagttacagttcttttgccttgacttcacttgaactagctctgtagaccaggttggcctcaaagactcaggcgtccacttgcctcccaaagtgctgggattaaaggtgtgccatttcaggcatcagccagagatttaaaaagaaaaaaaaaaatccagatataaaaagaatactgtttatctccacttttagaaaattggggagggatatagtgcagagatagagtacctagcatgtgggtggtcctggattgaagagagtagtctgcaagagaaaaagcaaaagagattctctaatagaggaaggactccatgagaagagtagaaagaaggttctagaacaaccaaggcagggagtctccagacagaatgagcacagtgtgccgagaaggaaaaggccaaggcccgtgactgagatccttgagggtctgtaggtgctgcacagttatggaaatgatatccagggtccagcctctcagttctgaagactgggtaactgcctctgtatccatcagcttctgccaagatggagtgatgagtctgtgttcaaaccgattcttccttctggccaagcatacggtgggtgtggtgcactggtgtgtgcacgtgagtagtaagtgagtgcgttccttgttccttcagcccaggagcttctgggtcctttggaaacaattgcaggagaccagaataacacagctcattcatcactgggtatggctcagcccagcatcttgtgtcagaaacgatccgaggggcatgtttgatggggattagcgctgctctcaatatcctgccttttggtcccagctgactctgacattccttgctggtgagttcctgctcatttcagttctagccctggagcagcagtcagtattaaagcaaacacttattctgtgtgtcacagtgccaaggggaactatcactattctgatagtatgttaacacgcgttgggtgcaatcttaccagagccttaccccatagcttctaggtatgcactatatagttcattgtcttgaaacctgatgtagcttcctcaaaagtcactatggaaatggactgacacaagccaccggccagtgggagaatggggtttgaagcttagcgactgtaagttcatcatgtaatgtaggagggggaagtgtggtgttagtaatacctatagtcttagttcttgtggagttgaggcaggaaaatcccttgagcctaagtgaagatgaggtaacatagcaggaccctgtgtctgtgtgtgtgtgtgtgtgtgtgtgtgtgtgtgtgtgtgtgtgtgtgtgtgactgactgagatagtctcaatatatgtagccttgtctgccctggaactcttctcatggccttgaactcctagagatctttttgcctctgtttctgcttctgccttccatgtgctgggactaaaggcatatgccaccatgccaggtcccaggtccctgtctcttttaaaaaataaagcagaaagaaacaccatacttggggctggagagatggctcagcggttaagagcactgactgttcttatgaaggtcctgagttcaaatcccaacaaccacatgatggcttacaaccatctgtaacgaaatctgatgctctcttctggagtgtctgaagacacactacagtgtacttacatataataaataaataaataaataaataaataaataaatattaaaaaaaaagaaacaccacacttaaactgatgccttacaagaacttcgtacgtgttttgtacctgttactgtgcagctgtggtcgggacaagggttatcagagagagatctatctggtagagtttgagctctagatgtcagtggatgtggcatgtcacgggcttcggctttgtgccttcctactttgcagctgggtgtaattttctgcattcacctggtgtttcttgcagcagaaatcatcacacccataagagaagtagcctctgagttaatcttaagtgaggtagacctctgggctatgcttaagttgtccgctaattcatcacttgtgctggaaggaatgtgcactctccaaacagatgctgtagaggactgttatctccaccttgcagtgaagtgaactgaggttctgagatttaaggacaaaggttcctgtcgggggaaggacagggagaggtgtgtgtggggggtgggactgggagagaaagggacaggcaggtttgagacagggcttctcagtgtagcctaggcttacctggaattcacagagatccacctgtctctgagtcatgggtgctggacatttgtttattttaaagatttatttatttattttatatgagtacagtgtagctgtcttcagacacactagaagagggcatcagataccattacagttggttgtgagtcaccatgtggttgctgggaattgaactcgggacctccggtagagcagtcagtgctcttaactgctgagccacctctccagcccaggatatttgttttaagagtgatagaactaggaagctggaggaatagggcagtcgataaagtgcttaccttaagttcaaagacatgcttttctgctgctgcttgtcctcctcctccccttccctctttctcttcttcctcctcctcctcttttctttcaagatagggtctctctctacatagctcttgctgtcctagaaccctatctgtaaaccaggctagcctcaaactcttacctgctcccgactcctgaggagacaggttctcaaacagacccttttgagacgggctggacttgaactcttgaccttcctgccttacttattctgctagtgctggcaccacaggcagctgcctggttttgtgttagacctttttagtggtggtcagaggtgccttgggcagcagctgcagagccacgtgggcatagggaaaatgcccaggggcagggcagaggccatcagggcagcggagtgagcagctctcatggcacgtcagcctccatcgtgtcctcggtgtgcacatggttggatgctcgctttgagttcttctgtaggatcttcccttatccccaaatgttttagttataaaaaaaaaaaaaaaaaaaaaaaaacaacttcccagaggtgcaaggagccacctccttctgcagttcattctgtgaatggagcttgttgctttgtgtcaggcatttgcaagtgcgggtcactcagaggttgagaagtcctcaccctggtgcttcctggaggctggggggaggagggcagcgggatggggggaaaggttggcctcccaagtgctgcaatgaaagtcaggcaccactacacctggctaattttatttttaaaaacttctgtatatgtgtggccatgcacacatttagaggccagaaggtgtcggatccccagaggtggagttacagatggtcatgagccacctaacatgggtgctgggaaccgaatgtgggtcctctgcaagaacagtgcacacctttgactgctgagccatctttccagccccagcttgttcaattccaaacaataagtcagtatttaggaaaacaaagcattccatgagtcaggataagatgcctgatgccccttctgaaccatgctgcccgttctcatgggtaggagatttacagtatcatttgctgtctagtccatattcaaacacccccaactatcccaagaatgttttatttagtgtaggaacttaaacgcaaggcatctcagacatgacctccaattctgtacagtcaagaatgtaaagaaggggaggtagtcctgcctttgatcagcctgcctatccctctttcttctccctcttgggggttgagccaggaagcatcttattggattataggagtcaagattctggttttgtgtttttaagcacatggatttcatataagctacatctatgcgcacaaatagccacacacagagctcaagtttgtaatttaaaaagacatataatgtctccccctgtaataaaggcaagctggcgtttctggatctgaagtgtctggtagtgtctgcatgttacttccacaaggaacaaagggctttgatgcagatgctgctgcagttagtcatgaaggtcaagcccccaggtctgtctgtcctttacagacttctttcctcaaagcgggcaatctaggagagaaagctgattctgtttgtccctggaggcaggcctggagctcaactgtatagaccaggctggcctcagacgtgtggcagttctgcctgggcctcacgtgtggtggattgttttccatccaggtttggagtcacaggcttgtaatcataaaagttggaagtgcagttagtacactgcagaatggtttagcagcacagcatacttactgctcagcagaggcaagaggaacgttggaagtttgaagctagcctagtccacacagtgagttctggtttatccagagctcaagctgtgcttgtctgctgtgtatgaaaccctggacttgttctgcagtgctgcataaactggtcatggtggtccattccagcacaaggacatttttgctaagtcgtaagcctaggctacatgaggcactgtctcaaaaaaaaattttttttttaaggtagagaataatagagaagggaatccagcatctacttctgcccaaacatatatatcttcttcactcccaggaacacacacgccacacctgaaaacagctattagcagtactaggcagggaactatatgcatacctcttagatggctgagtttaaggccaacctgggccacgcaggcaattcaaagccagccaaggctatgcatatatagtgaggctatctgggaaagatggaaaacaaatgaaacatcagactgaggcagagcgggacacttgacctctcacgtcacaagcttctggacttacagtgggtaacatcctgctaagttggaagtgcagttggaacactgcaaaatggtttagcaatacagcatagtgcccagtggccgagtgctgcaggctgcagactgctagcctgggacccagatgcaatctttatagtctccatcaaacatgtattacttgattttgaactgccgtatccatcacactggcggccgctcgagcccctccggaggagtggccagggcgttctggaggtagaaactggttattgctacacaagatggagactatatccaaaccactgaattttgtgttattaacagtagttttgattttaaaaattgattgccaaggacaaatactaattacttttaaatattacctaccagtttccttttgctttgcttttatgttttaaaaattatttattttatgtatatgagtacaatatagatgtcttcagaagagggcatcagagatctcattacagatggctgtgagccaccacgtggttgctgggaattgaactcaggacctctggaagagcagccagtgcttttagctgctgagccatctctccagtcccttgtttttgttattattatattttatctacttttgggtttgtgtctgtgcacacataccacgttgtgcacatggaggtttgaaaacatgagcgtcttctcctgccgtcattttacttggctgcatgcctttgcccgctgagccatcttgccagcctttttatttttgtttccttttgtccttgttgagataggttctcactatggagcccagactagctttgaactccctactccaacctgtcctatctctcaggcaatgacctaaaccaaccagtgagtcttcctgggcccacctaggtttttggcgcgccctacgtcacccgccccgttcccacgccccgcgccacgtcacaaactccaccccctcattatcatattggcttcaatccaaaataaggtatattattgatgatgttacatcg

SEQ ID NO: 75 - DNA, 4056 bp, Artificial Sequence
  pBest4
ggccgctgcggccgtttaaacgccagcaacgcggcctttttacggttcctggccttttgctggccttttgctcttaattaaatcgcatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtcgagtgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgatagggagacccaagctggtgagtttggggacccttgattgttctttctttttcgctattgtaaaattcatgttatatggagggggcaaagttttcagggtgttgtttagaacgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacaggggacccaagcttggtaccgagctcggatccactagtaacggccgccagtgtgctggaattctgcagatatccatcacactggcggccgctcgagcatgcatctagagggccctattcaattctttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcatcgatccataacttcgtataatgtatgctatacgaagttatccagatctcgatttaagggtgggaaagaatatataaggtgggggtcttatgtagtttaaacgctagcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc

SEQ ID NO: 76 - DNA, 5624 bp, Artificial Sequence
  pAd5pTP
ggatcccctgagggggcccccatgggctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttaccatggccttgagcgtcaacgattgcgcgcgcctgaccggccagagcgtcccgaccatggagcactttttgccgctgcgcaacatctggaaccgcgtccgcgactttccgcgcgcctccaccaccgccgccggcatcacctggatgtccaggtacatctacggatatcatcgccttatgttggaagatctcgcccccggagccccggccaccctacgctggcccctctaccgccagccgccgccgcactttttggtgggataccagtacctggtgcggacttgcaacgactacgtatttgactcgagggcttactcgcgtctcaggtacaccgagctctcgcagccgggtcaccagaccgttaactggtccgttatggccaactgcacttacaccatcaacacgggcgcataccaccgctttgtggacatggatgacttccagtctaccctcacgcaggtgcagcaggccatattagccgagcgcgttgtcgccgacctagccctgcttcagccgatgaggggcttcggggtcacacgcatgggaggaagagggcgccacctacggccaaactccgccgccgccgcagcgatagatgcaagagatgcaggacaagaggaaggagaagaagaagtgccggtagaaaggctcatgcaagactactacaaagacctgcgccgatgtcaaaacgaagcctggggcatggccgaccgcctgcgcattcagcaggccggacccaaggacatggtgcttctgtcgaccatccgccgtctcaagaccgcctactttaattacatcatcagcagcacctccgccagaaacaaccccgaccgccgcccgctgccgcccgccacggtgctcagcctaccttgcgactgtgactggttagacgcctttctcgagaggttttccgatccggtcgatgcggactcgctcaggtccctcggcggcggagtacctacacaacaattgttgagatgcatcgttagcgccgtatccctgccgcatggcagccccccgccaacccataaccgggacatgacgggcggcgtcttccaactgcgcccccgcgagaacggccgcgccgtcaccgagaccatgcgccgtcgccgcggggagatgatcgagcgctttgtcgaccgcctcccggtgcgccgtcgtcgccgccgtgtcccccctcccccaccgccgccagaagaagaagaaggggaggcccttatggaagaggagattgaagaagaagaagaggcccctgtagcctttgagcgcgaggtgcgcgacactgtcgccgagctcatccgtcttctggaggaggagttaaccgtgtcggcgcgcaactcccagtttttcaacttcgccgtggacttctacgaggccatggagcgccttgaggccttgggggatatcaacgaatccacgttgcgacgctgggttatgtacttcttcgtggcagaacacaccgccaccaccctcaactacctctttcagcgcctgcgaaactacgccgtcttcgcccggcacgtggagctcaatctcgcgcaggtggtcatgcgcgcccgcgatgccgaagggggcgtggtctacagccgcgtctggaacgagggaggcctcaacgccttctcgcagctcatggcccgcatttccaacgacctcgccgccaccgtggagcgagccggacgcggagatctccaggaggaagagatcgagcagttcatggccgagatcgcctatcaagacaactcaggagacgtgcaggagattttgcgccaggccgccgtcaacgacaccgaaattgattctgtcgaactctctttcaggttcaagctcaccgggcccgtcgtcttcacgcagaggcgccagattcaggagatcaaccgccgcgtcgtcgcgttcgccagcaacctacgcgcgcagcaccagctcctgcccgcgcgcggcgccgacgtgcccctgccccctctcccggcgggtccggagccccccctacctccgggggctcgcccgcgtcaccgcttttaggaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcatcccgcccctaactccgcccagttccgcccattctccgccccatggctgactaattttttttatttatgcagaggccgaggccgcctcggcctctgagctattccagaagtagtgaggaggcttttttggaggccggatcctgcccagatctgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacg

SEQ ID NO: 77 - DNA, 16392 bp, Artificial Sequence
  pGLAd4
ttaattaagcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcttaattaaatcgatttaagggtgggaaagaatatataaggtgggggtcttatgtagtttgagctcggctttggttatttgatggattgacctagttctcaggcagccacctgtgtggactaaatacacccatttcttctccattctgaaggatgtgctgaaagccgccttggacagtgagaaaagcaaacatggaagcctgctgtaggatcaccttacaccatcagatacccacttcccacccccggcttcctgaactttctgtaattactcatgccagtgactagggctcatgtgggccatcccaggaaggagactagtggctgccattctgtcctggtctttgtgtctcccgaggaggagtgtaggtggtgaggtcacaggctaagccaggccatttaaggaaagctgcctcagctccagcccagccctctctgccaggtggatatggaaatcccggtttactctctggggccttttattggctgaaatgctcaccttggtatctgttacacattcccttccaagagcagagctagataagccaggtgtggagacaatcagctcttgaaatctcccagaagctgccaaacccctaaaatcagcaagagacacccctcatagtccagactcagctgttttattctgcctctccctgatctcttttcagggcataaagttttccaaggagaagaatgaaatcagtatagcaggtcattgagaaagtatagaaaggctcaggttgaacttcagtgtctgctaccaatgtgtcataaataagtcattttactactgtgagactcagtttacccatttgttgagtatgaaaaaaaatcatatatatttgttatgctatatgtatcaatgcatcgagctagactctggggctaaagcaatgagtaaaacgtatttggctgaacataaatatgggaggggggtggaatgcatgaatgtaaagaatgctggcataagtaggaccagtgagttgggtgtggcagtgcaccttggagacagagccaggcggacctctgagttcaaggacagtcatcagggctccacagagaaatcctgtccaaaaaaaaaaaaaaaaatcagtggggaaaggcacctgccatcgaggctgaagacctgaatttggtccacaggatccacgtggtaggaaagaaagaacttccaagggttattctctaacctccatacttatatgtgagtgtacacttaagtaatgtttgtttgtttgttttgttcctgttatgttttgtttgtttttaaagccaagtctggtggttcaggcatttaatcatagcacccagaaggcagagttcaagggcagcctggtctacataggaaattgtaggcctatacagtgaaaccctgtttcaaaggaagattaaaggaaaaaagtcttgcactcttccacaccctgaccaaagtcctatagtgaaactctggtcccctccctcatcactgctattgccaaactcagtctccctctatgacccacatcatgatagccagggctagaccgtttgtaaagataaatcagccttgtctggataaataactccaggaaggagaaactgccggctgtaaagaaccttagcagtcgtattccccaaaaccacctatataagcctcaagtgcccctccacctttcacagacacccctcccctacgggagagtatctgtcctgcactgtttgaaataaacagtcctgtctgttgaggtcagtcttcagtctttttttctgcttccagtctcttaagttgcttcagaaatttaccaagaatcactaaataaagccagctgtgatggtccacatctgtattccagtgcttcgaaggctgaggcagagggtctcaagtgtgaggctagcctgggctgcctgtggaaccctgcctttttttggggagcaggcttttaaaaaatatttatcacggtgaaatataaggcaggaggattgttagaagttcaataaggacaatctgattctgtagtggggttcaggctagctagggctacatactaaacaaggtctcaaccttgttttagtaagaggaggaggaggaaacagaaggaaaaaaaagtaatttcttaggcacccagtaggcctgcaagtcttgttttgaacaaatgaatggatgcatgaatcaatctttgctacctttcaggctctattctgtgtccccagagcacccccggcccaggaaagtgagcagggcacggtaggtctgctggaatctctgattgtacttgtgagggctctgcctagcagtctccctggaagctgagcctggccttagcctcttccttctctctcagagcttctgaagccacagaagggacgcatcaaactcggacctagaaatgaacgattagtcttttttagtgattttttttcttccgtctttattttttgacactcactgggtagccttggctggcccagagctccctaggttggtcagagatctgcctgcctcctgcctgggtgttgatatacagggattcaccactctatgctcttacgagctggcttcgaactcatcaggcttagaatgtcgggtcgtctcacttatacctcctaagtgttggaataaccagtgtgcaccacccaagagcccctagctgggactcttgccaggcctgtcttctctaagccttagtatccccatctccaaaatgggtataaccatatgcatacgtatcctaagggcatataccaaaagttcagtgaatacagtagagtatgtaggaccccctgcgggctagcagtagaagcctttccctttccattatacccttggttctaaagttggttgaaagttcccctaagctctctcccgcccagcaaaccagtcaaaccagtttcctcattccccgggcttctgtactaagcaaacactgctttgcaaacggttgaccaggagcttgcctttgttagagaaggaagtccaatggtgttttcttccttgcaatgccgggagtgggtttgctgtgccttagaaagggttcaaactgggctggaagtagggccccagagtagctgggggttggggattcaggatggcctcaagtttgttgttccccaaaccctcactttttctagaactatttcataatacacatgcttaaggatacccatttgttgacccattgcaaaatgctctctgacccagcttctgagactggttcaccccccatggataagaattgagtcatctggtgccattcacaaatacagattaacctccctcccccaatttactgaacctcaaccaaaaaaagatccgcctgcctctgcctcccaagtgctaggattttaaagggccacggtaccacacggatggacacaacattcattttcctgacagaccaggagtcaggtcacaggtgtttgactagtaaacacccggtgccaaccagggtgtgggcatttggctatctaccctgaggcccaagatccaagagcacccgtggtccatggtgctaggggacccggaaatgtagccagtgcatcagagatccagcctctgtcctcagggaagttcagaaacatctctgcttactgagacttctgcagggtatttgggaagaggggccagcacagcaggcatgatcagttttacatgctgggcctttgtaagtaagaggatgtgtccaggcacagtaaaggaaagtctcccccaggaccttgggcctttttatccactggtgaggctttttttctctttctgttcccccctccccacctcccgggctttccagacagagtttccctgtgtattcattcctggctgtcctggaacttgctaggtagaccgggctggcctcgaactcagtatgaacaaatgtttacttatggctggcactgagacttcctcttcaccctgactcctcacccagagcttcccctgaacagaattctcttggttagccttctcaagatcttctgtggaaaaggcagagatagtcatggggtgtggctctgtggtcaggctaaacctaatgagtgtgaggccctgactttgattcctagcattgaggaaaagcagaaacaggaatcttacccaattttcctctgcctgttaccagactcaaggctgtcccgtggagcccattgcccttcctgagaaacccacttcctttcctgaggctaggaaagacatccctcatctctgtgcctcaatctcattgatttttgtggtgttaaaattcagttatttgtctttgagagggctcccctcccccctttgcctttcacatcctttcccttctttattgctatgcagaggaccctaactcctaggcaggccctctacccttgagatacagctccatcctcttgctttttaaaaacttataattcgagacagggtctcactgctacccaggctggttttgagcttactctgcagcctcctgcatcagcccaccgccttgtacgtgagatggtgggcctgagactcagcctcttgaccttcacatacatctgatacgtgctatctgaacaggcctgtcttactcagttttctgggtcctgttggggggtgaggtggggaggggagatcttcgttcaggcatggagaggtgggaaggaaaccagtccacacctctaacaagtgagttccaactaggacttgcttagggcacataaggcagacagacgtgggagatgtgagcggcggggaagcgatgatgcttagatgctaccacgaggacctgatgtggtgtgcctgaagatgaacctgttccccatgagccagctcataaatggggaccagcaccggcccctgtgggataattggcctcgtcctagataataattacttagctttgtaattaggagagcttgtaagaagccgaaagtgtttcggatttattttacctggacaagttcaagactaaactaacacctgttcctgtggtttggcatgcagagccatctgtttccaaaggaggtcctgcagcagctttttttggggttctcctccctgttccttcccttctctccccacatcgccttttctccgtccagtggtcagtgtgtgtgtgtgtgtgtgtgtgtgtgtgtgtgttaatgagacagggtctcattatgtagccttgggcctggaactcactatgtagatgtggctggcttcaaactcagagaggcatatctatccacttgcctttgcctctgcctctcaagcggccgcgtgctgagactaaagggatgtgctactactcctggccagagtgataagtactcatcctgtttaaagttgcctgggattccctgtttgacactgaataaaaatcctgcccatgacttccaaggctctgggtccctccttcccccgcagcccatggcttgggccttcattttaaccccactccccacatgtgcttcgttcagatgctttgcttcctgattgtactcagaataccatggcagctcgggctgaggctggttttgaactcttaatactcctgtccgggagtagtagcatctcaggtgtgtccaatcacactctatgtattccttgtttgtgatgtctgtatcactcacccgtgcccttcaaagctttgctaagatgttgcctgggcaatgaggaccttctctttttgctttatttttctgtagccccccacctccccgttttttgggtggttttggtagggatgagacattaagatgttgcctgggcaatgaggaccttctctttttgctttatttttctgtagccccccacctccccgttttttgggtggttttggtagggatgagacaacagggccttatgcaggttatgcaagtaccctattgctgatccacatcccagcctttctggtattttccaccattgaacctgtcatgtggtttgtgtatttcctcagttcatagtcttcttccataaaaacataaatgtggccggacgtggtggcgcatgcctttaatcccagcactcgggaggcagaggcaggtggatttctgagttcgaggccagcctggtctacaaagtgagttccaggacagccagggctatagagaaaccctgtctcaaaaccaaaaaaaaaaaaaaaaaaaaaaaaaaaccaaaaccataaatgtgaggaggtcagggtgttctgtgtttgttggttcttgagctgtacttcaggccgtaacaagttcttagacctcatctgttgaatgagtgaacggaggagtgagaggtccagaaggtaaatatggcgctgtacagttctttttagaagctgtgctgtgggttttgttttttctaagataaaatggggtccagtgacatggctcagagtaaatgtaaatgttgtaccttttggcatcctgggttttatttttccagaacccatttatcctgtgacttccagatgcgcaccataccccctgtaaacccccccccccccaacacacacacacaccaaactagcaaacagatacagggctgtcagcgtgtgagcacatgaggaagggatgctatgttctcaggccctgtgtttcttcttggcaccccctggtttctctgagccctgcacagctacagtgtaaccttacctaattgtcttgttacaaagaagtttgctgcccagagtctaaaggtcagatgagtcacttgggcctcacacttctgggtgtatggaagcaccagatggaagtctcaaggtacacccgacaccggctgcctttccgtcgtcaggcagcacattcctgaagcaggctctctcagctgtgccctctcaaccttcatcctgcagcccattaattgttcaaggccttctagttcttggcaggtctgagcatcttggaaggagctagaagagagatgtagaaagctcaaagacaaaaatgcaaagctacccccatcagataccagctcttgggtctattgaccagccaatatccattgatagtggagttctacatccatatggaagatacaccattctctttcatggacaggagtgccaaatcagaatacaggacattacatctttttgagacaaggtcttaccatgtagcctttgtagaccagctatgtagaccatgctgccctggaacccacagagatttgctagcttctgcctcccaagtgctaagattagtgacacacacctccacttacacagtacacacccaggaaaaaagtaaaacataaataaaaagcaaaaaattgttgtgaaatagctcagtattagagtccttgtctaagatatatgggctccaggcttgcccatccatctattttctgagactggatttttctgtgtagccctggctgtcctgggtcttactttgtagaccaggctgaccttgaactcagagatccacctgcctctgcctctggagctctggggttaaaggtagaccccaccagccatgggcttaagcccagtatgggtttaaaaacatggaaccgattggactttgcataaagttagattggatcctgtgggtgctgacctcccatgctttcaagcacttattggtagcattcactgaacaagcctgagggcgggaacagccctggcatctttcaaagggtatccggttcaaaccagaatcgaatgctcttttcctagccaccccacctcttggcttgttgcctaaggaaaatggctctagcctggttctccagtgttctcatggcagtctggagagattgtccatttgtatcacaaccttttgggcgctgcccttggtgacccgctgctgatgtggacaccagcaagacttgagactgcagacctcctcccccacagatttctcacaatgcccgtgtgggagtgtttgaaaagggaacaagctgaccaacctgtttgtgggaaagtcgtggctttcctatcagaaaacgtcacactctgagtcaggagtgggacatacctgggtactttaacaggtggggcagggtcctgagtcatagagaggtctcacctgggaaggtttctctttcaaagagtgaatcaaccatgacatcacccatctggatctacaccctttcaagccgtctctctccttagcaaacaggaatctaaaagaggaataataaggtgaaagcaaactggaaataggacagaacagacaagggaaaaagttaaagagaaagcttaagagacgcagacacagacgtttacatgcaaacagggggtccataaaaacaaaaccggaagccgtaatatatgtacatggcctgtaagattagaaacaaatgtcctggcacaacattatgggacaacttcccaagctgctgttgagtttgttttgtgttggccggcatgggcccttaaactgagtggtgtgtttctctcttagagaatttttcatgaactagtttttcatttgcaagtggttagtagtttgagatagcttctaggttaggggtggggccgtgtgtccactttactcagctctaatgtcccccgtttggtgcagacccctgcagaccctatgcgtcctgccacagtctctgagttcatgcctgcattgttcaaattgtgtttccttgatgtcctccaatctcctctttgctgggctccctggggcccccaaggtgagggatttggcagaggtattcattccatttagggttgattgttccaaggtccctcactgtacgtgttatttgtcccttctccctggcttcctgctcaggagttctgcagacatggctggtgtttagtagcttcttggtgaagttcaggtagccagtttcatgaccacccagctctcaggcctgactaagcccgtggagaaagcccataaaataaagggggtgaacttctaatctacatctgaaaggagaacccctgtctacatctacctgttaccctagccatcggggagactgaggcagaaggatctagcctggaaatttagggtgagtctgggcagcattaggagactatttcacagtgaagaaggaaggaactcactcacacccttggtgcacctcacaggtctctgggctcagattggagtctttggttttgaaatttcctcatcatcggccgcctcagctggtttttcttctggagtctgttacgccttctcccctttccccccttctcttgctaatcctttcgcttgaacaataggagtccttaaaacctgggagggaagtaaatacctttcagatgagaccctgtcttaagagggggaaaaaaaaaacaaaaaaaccctagggaccgggtcaccaagtgtcaggttaatcagcagatgtcaagatgactcctgagtgcttgctctttcttcagctggtgaaattacagcggcaacatagaatgacagtcagcagggagggaggagagggcctgggtggccaaataaacttataaattgtgagagaaattaatgaatgtctaagttaatgcagaaacggagagacatactatattcatgaactaaaagacttaatattgtgaaggtatactttctttccacataaatttgtagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagccattttcatatttttcttgaacattaagtacaagtctataattaaaaattttttaaatgtagtctggaacattgccagaaacagaagtacaacagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaaagagattgtagaaagttgatacaatttttttttctttaaatatgtcgacgcactggctttacaggggccgtctgctttgtctgtagatatggtgataggagtctgatttagaaacttccttgttctcctcttcccgaatccttcctcctgaacaataggaatacctaagtgcacaagtgcaggtgcccaggaggccctgagacaaccccccctccccagctgcgagccttctccggtggtgctgggaatcgaactggagtcctctgcaagaacagtatgtgctcttaaccactgcaccatctcttcagccccctctagttctttagtttgtacataaattagcaagttcagggaatgcagaagaactggccaggggtcagagccagactggaaacatttttcaccaaacaatagctacagtgttcatggcgtgcctccattgaatactaaactagctgttgatgtgtgactttgatttttaatctttgcaccaactttagttactatatagataattccatttccaaatgatatatagtggaagcttgtaaaagctgcttactatcccagggtccccagccgagatctacatgataaagctactctcaacccagcctacattccccctccctgtgctcagccctacctggggttgttcactttcaagctcccaggaaggaacctgagcgctccctgcccccatgcagtccatttctatctctggtcctttgggtacttggagcctcaagctcctgactgctaatgaagcctgtaacagaagggcattacttactgcccgccttgtgctcccttgaaatctggtcacctggaggtggaagtgggcagtgcttccgtgcttccgtgcttccgctttctcccagctccttttaatcactattttttttttttcttttccaaattactacctaggagttttctagtaatcactttggtgggagtgggagggggagtggaggcagttaactctgtgagtgccaaggacagttgttggacggtggtagtgatggaggggagggtgttcacggtgagggaactctgtggagttacgtagctggcaacaatggggagagacggagccgcaccaccacagacgctttgcatgagcaactattttgttttctgtagatgttttccaaaccttaaactctgttaagtctgaaactgggaggggaacctctccaccaaaatctaaatatggatcagagataaggaaacttccatagtctccagctccctccctgactcagtggcctctgtgaccaactccacgtgactctagtggacaccttcctggtggtttggctttttttttttttccccatttcatcttttaaatggagttttccatgaataagaatataaaactgcgggggctggtgagatggatcagcaggtaagagcacagaggtcctgagttcaaatcccagcaaccacttggtggctcaccatcggtaacgagatctgactccctcttctagagtgtctgaagatagctacagtgtacttacatataataaataaacaaatcttaaaaaaaagaatataaaactgcttccttcccaacaggccttggatagttttgtctgtgggaacagcttcccataggaaaggtgcttttgctacttccgaccgtcacacactcctcctctgcaccccgtgagggcctcccaggagttttgtgttggtcacaagcctccccatgtcccaagggggtctggagagggatctgaggtggtggaaagtgctggggtgccaagttatcagacaatgggcacattgagaatgcaggtggggccaggcctgtgtgggctggagcagctgtcggagcctcagggggtgcagctcatgcgcagagggaagaggcagtctctctgctggctgagaggggagggccacagtaagcaaaggaggggggtgatccccccctcacaagattccagagcaaagggatgtttgaacgatgactcacatgacaggggcggggtctgtgagcaaacttcctgttggaagcttgttcttggttcctgactgtccttcccaggtcatagagactgtctgcaatagggctggggcctgttaggatgccatcgaggtgggaaatcctggagagcttggagtagccagcaggccctcttccctctcctgccccaggctagctggaaacccgcgaggaggaagggtgtgttgttccctatcccatggagaggcttgcatggtcagaccaggcctggtgtgacctctggggctgctctgtgactgccctgttctgtgactgcccttctccctcccagccctccccttccatttgcttctagattcgtctaggagcccatctcagcaaatggcagcatcctcgcttaccagacatcttgtttgtttgtttgtttgttcattcattctgggcctgaagatggagagctgtggtaggggagagctcagaggctgggggcagccttaagggcacagccaggacggagcctctgagcaaagggcagtgtgtgtagagtcaggtaggtggtgggcagcaggagacaggaggagcacagaggtaggtggcactgtacagaggccctggcaccattttgtgacaaggactctcagacccctctagcctattcccccacccccacccccattacaagcagtagggtcttaatagctaatggcttggacaccagcctctggctgctctggttcccagctggtcagagcccttgttctcctttgggttctgatccaggtggcacagggtgggggccctgattcacattcttgatagtgccgctaggttccctgctaccagctggcacattctgtcttcctttccagcaggtacttggctgggcttcctgcccagctgctagacctactgttaggtcagctcattggatgcttcctccaggaagccttctctaatctccattcccttggcttcttttcaagagcagaagttcactggcttctcctgtttccccagagtagcacctggtgtctcaagtactcgctaagcagttaggagaggtgtgaactgggcctcagggaccccagagacaggaggggaggtcattacaggaaagtcggattcttttcctggctttgttcttataaccatcgtgtgtgtgtgtgtgtgtgtgtgtgtgtgtgtgtgtgtgtgtgtgtgtgtgtacgtgtgtgtacgtgtgtgtgtgtgtacatgcgtgcgcgccctacgtcacccgccccgttcccacgccccgcgccacgtcacaaactccaccccctcattatcatattggcttcaatccaaaataaggtatattattgatgatgttacatcg

SEQ ID NO: 78 - DNA, 30119 bp, Artificial Sequence
  pGLAd4_coHTT.HTTmshR1/3
ttaattaagcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcttaattaaatcgcatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtcgagtgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgatagggagacccaagctggtgagtttggggacccttgattgttctttctttttcgctattgtaaaattcatgttatatggagggggcaaagttttcagggtgttgtttagaacgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacaggggacccaagcttggtaccgagctcggatccaccatggctactctggaaaaactgatgaaggcatttgagtctctgaagagcttccagcaacaacagcaacaacagcaacagcaacagcagcaacaacagcagcaacagcaacaacaacagccacctcctcctccacctccacctccaccccctcagctgccccagccaccacctcaggcacagcctctgctgccacagccccagccaccccctcctcctcctccaccacctccaggaccagcagtggccgaggagcctctgcacagaccaaagaaggagctgagcgccacaaagaaggatagggtgaaccactgtctgaccatctgcgagaatatcgtggcccagtctgtgaggaacagccctgagtttcagaagctgctgggcatcgccatggagctgttcctgctgtgctctgacgatgccgagagcgatgtgcggatggtggccgacgagtgtctgaacaaagtgatcaaggccctgatggacagcaacctgcccagactgcagctggagctgtacaaggagatcaagaagaatggagcacctaggtccctgagggccgccctgtggcgcttcgccgagctggcccacctggtgcggcctcagaagtgtagaccatatctggtgaacctgctgccctgcctgacacggacctccaagagacctgaggagtctgtgcaggagaccctggcagcagcagtgcctaagatcatggccagctttggcaatttcgccaacgacaatgagatcaaggtgctgctgaaggcctttatcgccaacctgaagagctcctctccaaccatcaggagaacagcagcaggaagcgccgtgtccatctgccagcacagcaggcgcacccagtacttctattcctggctgctgaatgtgctgctgggactgctggtgccagtggaggatgagcactccacactgctgatcctgggcgtgctgctgaccctgcggtacctggtgcctctgctgcagcagcaggtgaaggacacatccctgaagggctcttttggcgtgaccagaaaggagatggaggtgtccccctctgccgagcagctggtgcaggtgtatgagctgaccctgcaccacacacagcaccaggaccacaacgtggtgacaggcgccctggagctgctgcagcagctgttccggaccccacctccagagctgctgcagacactgaccgcagtgggaggaatcggacagctgaccgcagcaaaggaggagagcggaggccggagcagatccggatctatcgtggagctgatcgcaggaggaggaagctcctgcagccccgtgctgtccaggaagcagaagggcaaggtgctgctgggagaggaggaggccctggaggacgatagcgagtcccgctctgacgtgagcagctccgccctgacagccagcgtgaaggacgagatctccggagagctggcagcatctagcggcgtgtctacccctggaagcgccggccacgacatcatcacagagcagccaagaagccagcacaccctgcaggccgattccgtggacctggcctcttgtgatctgacctcctctgccacagatggcgacgaggaggacatcctgagccacagctcctctcaggtgtccgccgtgccttctgatccagccatggatctgaatgacggaacccaggccagctccccaatctccgactctagccagaccacaaccgagggccctgatagcgccgtgacaccatccgactcctctgagatcgtgctggatggcaccgacaaccagtacctgggactgcagatcggacagccacaggatgaggacgaggaggcaaccggcatcctgcctgatgaggccagcgaggcctttcggaatagctccatggcactgcagcaggcacacctgctgaagaacatgtctcactgcagacagcctagcgattctagcgtggacaagttcgtgctgagggatgaggcaacagagccaggcgaccaggagaataagccttgccggatcaagggcgacatcggccagtctaccgacgatgacagcgccccactggtgcactgcgtgcggctgctgagcgcctccttcctgctgaccggaggcaagaacgtgctggtgcccgatcgggacgtgagagtgtctgtgaaggccctggccctgagctgcgtgggagcagccgtggccctgcaccctgagtctttctttagcaagctgtacaaggtgccactggatacaaccgagtaccccgaggagcagtatgtgagcgacatcctgaattatatcgatcacggcgacccacaggtgcggggagcaaccgccatcctgtgcggcacactgatctgctctatcctgtctaggagccgctttcacgtgggcgactggatgggcacaatcaggacactgaccggcaacaccttctccctggccgattgcatccccctgctgcgcaagacactgaaggacgagtcctctgtgacctgtaagctggcctgcacagccgtgagaaattgcgtgatgtccctgtgcagctcctcttactctgagctgggcctgcagctgatcatcgatgtgctgaccctgagaaacagctcctattggctggtgaggaccgagctgctggagacactggccgagatcgactttagactggtgagcttcctggaggccaaggccgagaatctgcacaggggagcacaccactacaccggactgctgaagctgcaggagcgcgtgctgaacaacgtggtcatccacctgctgggcgatgaggacccacgggtgagacacgtggcagcagccagcctgatcaggctggtgcctaagctgttttacaagtgtgatcagggacaggcagacccagtggtggcagtggcaagggatcagtctagcgtgtatctgaagctgctgatgcacgagacccagcccccttcccacttctccgtgtctacaatcaccaggatctaccgcggctataacctgctgccctccatcacagacgtgaccatggagaacaatctgtctagagtgatcgccgccgtgtcccacgagctgatcacctctacaacccgcgccctgacatttggctgctgtgaggccctgtgcctgctgtccacagccttccccgtgtgcatctggtctctgggatggcactgcggagtgccaccactgagcgcctccgatgagtccaggaagtcttgtaccgtgggcatggccaccatgatcctgacactgctgtcctctgcctggtttccactggatctgagcgcccaccaggacgcactgatcctggcaggaaatctgctggcagcaagcgccccaaagtccctgcggagctcctgggcatccgaggaggaggcaaacccagcagcaaccaagcaggaggaagtgtggccagccctgggcgacagagccctggtgccaatggtggagcagctgttcagccacctgctgaaagtgatcaacatctgcgcacacgtgctggatgacgtggcacctggaccagccatcaaggccgccctgccttccctgacaaaccctccatctctgagcccaatccggagaaagggcaaggagaaggagccaggagagcaggcctctgtgccactgagccctaagaagggaagcgaggcctccgccgcatctaggcagagcgacacctccggcccagtgacaaccagcaagtctagctccctgggcagcttttaccacctgccctcctatctgaagctgcacgatgtgctgaaggccacccacgccaattacaaggtgacactggacctgcagaactccaccgagaagtttggcggcttcctgaggtctgccctggatgtgctgagccagatcctggagctggccacactgcaggacatcggcaagtgcgtggaggagatcctgggctatctgaagtcttgcttcagccgcgagccaatgatggccaccgtgtgcgtgcagcagctgctgaagaccctgtttggcacaaatctggccagccagttcgatggcctgtctagcaacccatccaagtctcagggaagggcacagagactgggatcctctagcgtgaggcctggcctgtaccactattgctttatggccccatacacacacttcacccaggccctggcagacgcctccctgcgcaatatggtgcaggccgagcaggagaacgatacatctggctggtttgacgtgctgcagaaggtgagcacccagctgaagacaaacctgacctccgtgacaaagaatagagccgataagaacgccatccacaatcacatcaggctgttcgagcccctggtcatcaaggccctgaagcagtatacaaccacaacctgcgtgcagctgcagaagcaggtgctggacctgctggcccagctggtgcagctgagggtgaattactgtctgctggattccgaccaggtgtttatcggcttcgtgctgaagcagtttgagtatatcgaagtgggccagttccgggagtctgaggccatcatccctaacatcttctttttcctggtgctgctgtcctacgagagatatcactctaagcagatcatcggcatcccaaagatcatccagctgtgcgacggcatcatggcaagcggaaggaaggcagtgacccacgcaatccccgccctgcagcctatcgtgcacgatctgtttgtgctgcgcggcacaaataaggcagacgcaggcaaggagctggagacccagaaggaggtggtggtgtccatgctgctgcgcctgatccagtaccaccaggtgctggagatgttcatcctggtgctgcagcagtgtcacaaggagaacgaggataagtggaagaggctgtcccgccagatcgccgacatcatcctgcccatgctggccaagcagcagatgcacatcgattctcacgaggccctgggcgtgctgaataccctgtttgagatcctggcaccatcctctctgaggcctgtggacatgctgctgagatctatgttcgtgacccctaacacaatggccagcgtgtccacagtgcagctgtggatcagcggcatcctggccatcctgcgggtgctgatctctcagagcaccgaggatatcgtgctgagcagaatccaggagctgtccttttctccatacctgatctcctgcaccgtgatcaataggctgcgcgatggcgacagcacatccaccctggaggagcacagcgagggcaagcagatcaagaacctgcccgaggagacattttcccggttcctgctgcagctggtgggcatcctgctggaggacatcgtgaccaagcagctgaaggtggagatgagcgagcagcagcacacattctattgtcaggagctgggcaccctgctgatgtgcctgatccacatctttaagtccggaatgttcaggaggatcaccgcagcagcaacaaggctgtttagaagcgatggctgcggcggctccttctacacactggactctctgaatctgcgggccagaagcatgatcacaacccaccccgccctggtgctgctgtggtgtcagatcctgctgctggtgaaccacaccgactataggtggtgggcagaggtgcagcagacaccaaagaggcacagcctgagctccaccaagctgctgtcccctcagatgtctggagaggaggaggattccgacctggcagcaaagctgggcatgtgcaacagagagatcgtgcggagaggcgccctgatcctgttctgtgattacgtgtgccagaatctgcacgacagcgagcacctgacctggctgatcgtgaaccacatccaggatctgatctctctgagccacgagccccctgtgcaggactttatctccgccgtgcaccggaacagcgccgcatctggcctgttcatccaggccatccagagcagatgcgagaacctgtccacacccaccatgctgaagaagaccctgcagtgtctggagggcatccacctgtcccagtctggcgccgtgctgacactgtatgtggataggctgctgtgcaccccctttagggtgctggcccgcatggtggacatcctggcctgtaggcgcgtggagatgctgctggccgccaatctgcagtctagcatggcccagctgcctatggaggagctgaaccgcatccaggagtacctgcagtcctctggactggcacagaggcaccagcgcctgtattctctgctggataggttccgcctgagcaccatgcaggacagcctgtccccatctccacccgtgagctcccacccactggatggcgacggacacgtgtccctggagacagtgtctcccgataaggactggtacgtgcacctggtgaagagccagtgctggaccaggagcgattccgccctgctggagggagcagagctggtgaatcgcatccctgccgaggacatgaacgcctttatgatgaacagcgagttcaatctgtccctgctggccccatgtctgtctctgggcatgagcgagatctccggaggacagaagagcgccctgtttgaggccgccagggaggtgacactggcccgcgtgtccggaaccgtgcagcagctgccagcagtgcaccacgtgtttcagccagagctgccagcagagcctgcagcatactggtctaagctgaacgatctgttcggcgacgccgccctgtaccagagcctgcctacactggcaagagccctggcacagtatctggtggtggtgagcaagctgccatcccacctgcacctgcctccagagaaggagaaggatatcgtgaagttcgtggtggccaccctggaggccctgagctggcacctgatccacgagcagatcccactgtccctggatctgcaggcaggactggactgctgttgcctggccctgcagctgccaggactgtggtctgtggtgtctagcaccgagtttgtgacacacgcctgtagcctgatctactgcgtgcacttcatcctggaggcagtggcagtgcagcctggagagcagctgctgtccccagagcggagaacaaatacccccaaggccatctctgaggaggaggaggaggtggacccaaacacacagaatcccaagtatatcaccgcagcatgcgagatggtggcagagatggtcgagtctctgcagagcgtgctggccctgggacacaagcggaacagcggagtgcctgccttcctgacaccactgctgcgcaatatcatcatctctctggcccggctgcccctggtgaacagctacaccagagtgccccctctggtgtggaagctgggatggtccccaaagcctggaggcgattttggcaccgccttcccagagatccccgtggagtttctgcaggagaaggaggtgtttaaggagttcatctatcgcatcaatacactgggctggaccagccggacacagttcgaggagacctgggccacactgctgggcgtgctggtgacccagcctctggtcatggagcaggaggagtccccaccagaggaggacaccgagaggacacagatcaacgtgctggccgtgcaggccatcacatctctggtgctgagcgccatgaccgtgccagtggcaggaaatcctgccgtgagctgcctggagcagcagccaagaaacaagcccctgaaggccctggatacacggttcggcagaaagctgtccatcatccggggcatcgtggagcaggagatccaggccatggtgtctaagagagagaacatcgccacccaccacctgtaccaggcctgggacccagtgccctctctgagcccagcaacaaccggcgccctgatcagccacgagaagctgctgctgcagatcaatcctgagagggagctgggctccatgtcttataagctgggccaggtgtccatccacagcgtgtggctgggcaactccatcacacccctgcgcgaggaggagtgggatgaagaggaggaggaggaggcagacgcacctgcaccatcctctcctccaacctcccctgtgaacagcaggaagcaccgcgccggcgtggatatccacagctgttcccagttcctgctggagctgtacagcagatggatcctgcctagctcctctgccaggcgcacaccagccatcctgatctctgaggtggtgcgcagcctgctggtggtgtccgacctgtttaccgagcggaaccagttcgagctgatgtacgtgacactgaccgagctgcggagagtgcacccaagcgaggatgagatcctggcccagtatctggtgccagcaacatgcaaggcagcagccgtgctgggaatggacaaggcagtggcagagcccgtgagcaggctgctggagagcaccctgcgcagctcccacctgccatccagggtgggcgccctgcacggcgtgctgtacgtgctggagtgtgatctgctggatgacaccgccaagcagctgatccccgtgatctccgactatctgctgtctaatctgaagggcatcgcccactgcgtgaacatccacagccagcagcacgtgctggtcatgtgcgccacagccttttacctgatcgagaactatccactggacgtgggccccgagttctctgccagcatcatccagatgtgcggcgtgatgctgtccggctctgaggagagcaccccatccatcatctaccactgtgcactgaggggactggagagactgctgctgagcgagcagctgtccaggctggatgccgagagcctggtgaagctgtccgtggacagagtgaatgtgcacagcccacacagggcaatggccgccctgggactgatgctgacctgcatgtatacaggcaaggagaaggtgtctccaggaaggaccagcgatccaaaccctgcagcacctgacagcgagtccgtgatcgtggccatggagcgcgtgtccgtgctgtttgatcggatcagaaagggcttcccatgtgaggcaagggtggtggcacgcatcctgccacagtttctggatgactttttcccccctcaggacatcatgaacaaagtgatcggcgagttcctgtctaaccagcagccatacccccagtttatggccaccgtggtgtataaggtgttccagaccctgcacagcacaggccagtctagcatggtgcgcgattgggtcatgctgtctctgagcaacttcacccagagggcacctgtggcaatggcaacctggtccctgtcttgctttttcgtgagcgcctccacatctccttgggtggccgccatcctgccacacgtgatcagccggatgggcaagctggagcaggtggatgtgaacctgttttgtctggtggccaccgacttctacagacaccagatcgaggaggagctggacaggagggcattccagtccgtgctggaggtggtggccgccccaggctccccataccataggctgctgacctgtctgaggaatgtgcataaagtgactacctgttgatgaattctgcagatatccatcacactggcggccgctcgagcatgcatctagagggccctattcaattctttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcatcgatccataacttcgtataatgtatgctatacgaagttatccagatctcgatttaagggtgggaaagaatatataaggtgggggtcttatgtagtttgagctcggctttggttatttgatggattgacctagttctcaggcagccacctgtgtggactaaatacacccatttcttctccattctgaaggatgtgctgaaagccgccttggacagtgagaaaagcaaacatggaagcctgctgtaggatcaccttacaccatcagatacccacttcccacccccggcttcctgaactttctgtaattactcatgccagtgactagggctcatgtgggccatcccaggaaggagactagtggctgccattctgtcctggtctttgtgtctcccgaggaggagtgtaggtggtgaggtcacaggctaagccaggccatttaaggaaagctgcctcagctccagcccagccctctctgccaggtggatatggaaatcccggtttactctctggggccttttattggctgaaatgctcaccttggtatctgttacacattcccttccaagagcagagctagataagccaggtgtggagacaatcagctcttgaaatctcccagaagctgccaaacccctaaaatcagcaagagacacccctcatagtccagactcagctgttttattctgcctctccctgatctcttttcagggcataaagttttccaaggagaagaatgaaatcagtatagcaggtcattgagaaagtatagaaaggctcaggttgaacttcagtgtctgctaccaatgtgtcataaataagtcattttactactgtgagactcagtttacccatttgttgagtatgaaaaaaaatcatatatatttgttatgctatatgtatcaatgcatcgagctagactctggggctaaagcaatgagtaaaacgtatttggctgaacataaatatgggaggggggtggaatgcatgaatgtaaagaatgctggcataagtaggaccagtgagttgggtgtggcagtgcaccttggagacagagccaggcggacctctgagttcaaggacagtcatcagggctccacagagaaatcctgtccaaaaaaaaaaaaaaaaatcagtggggaaaggcacctgccatcgaggctgaagacctgaatttggtccacaggatccacgtggtaggaaagaaagaacttccaagggttattctctaacctccatacttatatgtgagtgtacacttaagtaatgtttgtttgtttgttttgttcctgttatgttttgtttgtttttaaagccaagtctggtggttcaggcatttaatcatagcacccagaaggcagagttcaagggcagcctggtctacataggaaattgtaggcctatacagtgaaaccctgtttcaaaggaagattaaaggaaaaaagtcttgcactcttccacaccctgaccaaagtcctatagtgaaactctggtcccctccctcatcactgctattgccaaactcagtctccctctatgacccacatcatgatagccagggctagaccgtttgtaaagataaatcagccttgtctggataaataactccaggaaggagaaactgccggctgtaaagaaccttagcagtcgtattccccaaaaccacctatataagcctcaagtgcccctccacctttcacagacacccctcccctacgggagagtatctgtcctgcactgtttgaaataaacagtcctgtctgttgaggtcagtcttcagtctttttttctgcttccagtctcttaagttgcttcagaaatttaccaagaatcactaaataaagccagctgtgatggtccacatctgtattccagtgcttcgaaggctgaggcagagggtctcaagtgtgaggctagcctgggctgcctgtggaaccctgcctttttttggggagcaggcttttaaaaaatatttatcacggtgaaatataaggcaggaggattgttagaagttcaataaggacaatctgattctgtagtggggttcaggctagctagggctacatactaaacaaggtctcaaccttgttttagtaagaggaggaggaggaaacagaaggaaaaaaaagtaatttcttaggcacccagtaggcctgcaagtcttgttttgaacaaatgaatggatgcatgaatcaatctttgctacctttcaggctctattctgtgtccccagagcacccccggcccaggaaagtgagcagggcacggtaggtctgctggaatctctgattgtacttgtgagggctctgcctagcagtctccctggaagctgagcctggccttagcctcttccttctctctcagagcttctgaagccacagaagggacgcatcaaactcggacctagaaatgaacgattagtcttttttagtgattttttttcttccgtctttattttttgacactcactgggtagccttggctggcccagagctccctaggttggtcagagatctgcctgcctcctgcctgggtgttgatatacagggattcaccactctatgctcttacgagctggcttcgaactcatcaggcttagaatgtcgggtcgtctcacttatacctcctaagtgttggaataaccagtgtgcaccacccaagagcccctagctgggactcttgccaggcctgtcttctctaagccttagtatccccatctccaaaatgggtataaccatatgcatacgtatcctaagggcatataccaaaagttcagtgaatacagtagagtatgtaggaccccctgcgggctagcagtagaagcctttccctttccattatacccttggttctaaagttggttgaaagttcccctaagctctctcccgcccagcaaaccagtcaaaccagtttcctcattccccgggcttctgtactaagcaaacactgctttgcaaacggttgaccaggagcttgcctttgttagagaaggaagtccaatggtgttttcttccttgcaatgccgggagtgggtttgctgtgccttagaaagggttcaaactgggctggaagtagggccccagagtagctgggggttggggattcaggatggcctcaagtttgttgttccccaaaccctcactttttctagaactatttcataatacacatgcttaaggatacccatttgttgacccattgcaaaatgctctctgacccagcttctgagactggttcaccccccatggataagaattgagtcatctggtgccattcacaaatacagattaacctccctcccccaatttactgaacctcaaccaaaaaaagatccgcctgcctctgcctcccaagtgctaggattttaaagggccacggtacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatcctgctgttgacagtgagcgagaccgtgtgaatcattgtctatagtgaagccacagatgtatagacaatgattcacacggtcgtgcctactgcctcggagaattccagcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtaccacacggatggacacaacattcattttcctgacagaccaggagtcaggtcacaggtgtttgactagtaaacacccggtgccaaccagggtgtgggcatttggctatctaccctgaggcccaagatccaagagcacccgtggtccatggtgctaggggacccggaaatgtagccagtgcatcagagatccagcctctgtcctcagggaagttcagaaacatctctgcttactgagacttctgcagggtatttgggaagaggggccagcacagcaggcatgatcagttttacatgctgggcctttgtaagtaagaggatgtgtccaggcacagtaaaggaaagtctcccccaggaccttgggcctttttatccactggtgaggctttttttctctttctgttcccccctccccacctcccgggctttccagacagagtttccctgtgtattcattcctggctgtcctggaacttgctaggtagaccgggctggcctcgaactcagtatgaacaaatgtttacttatggctggcactgagacttcctcttcaccctgactcctcacccagagcttcccctgaacagaattctcttggttagccttctcaagatcttctgtggaaaaggcagagatagtcatggggtgtggctctgtggtcaggctaaacctaatgagtgtgaggccctgactttgattcctagcattgaggaaaagcagaaacaggaatcttacccaattttcctctgcctgttaccagactcaaggctgtcccgtggagcccattgcccttcctgagaaacccacttcctttcctgaggctaggaaagacatccctcatctctgtgcctcaatctcattgatttttgtggtgttaaaattcagttatttgtctttgagagggctcccctcccccctttgcctttcacatcctttcccttctttattgctatgcagaggaccctaactcctaggcaggccctctacccttgagatacagctccatcctcttgctttttaaaaacttataattcgagacagggtctcactgctacccaggctggttttgagcttactctgcagcctcctgcatcagcccaccgccttgtacgtgagatggtgggcctgagactcagcctcttgaccttcacatacatctgatacgtgctatctgaacaggcctgtcttactcagttttctgggtcctgttggggggtgaggtggggaggggagatcttcgttcaggcatggagaggtgggaaggaaaccagtccacacctctaacaagtgagttccaactaggacttgcttagggcacataaggcagacagacgtgggagatgtgagcggcggggaagcgatgatgcttagatgctaccacgaggacctgatgtggtgtgcctgaagatgaacctgttccccatgagccagctcataaatggggaccagcaccggcccctgtgggataattggcctcgtcctagataataattacttagctttgtaattaggagagcttgtaagaagccgaaagtgtttcggatttattttacctggacaagttcaagactaaactaacacctgttcctgtggtttggcatgcagagccatctgtttccaaaggaggtcctgcagcagctttttttggggttctcctccctgttccttcccttctctccccacatcgccttttctccgtccagtggtcagtgtgtgtgtgtgtgtgtgtgtgtgtgtgtgtgttaatgagacagggtctcattatgtagccttgggcctggaactcactatgtagatgtggctggcttcaaactcagagaggcatatctatccacttgcctttgcctctgcctctcaagcggccgcgtgctgagactaaagggatgtgctactactcctggccagagtgataagtactcatcctgtttaaagttgcctgggattccctgtttgacactgaataaaaatcctgcccatgacttccaaggctctgggtccctccttcccccgcagcccatggcttgggccttcattttaaccccactccccacatggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatcctgctgttgacagtgagcgaggatacctgaaatcctgcttttagtgaagccacagatgtaaaagcaggatttcaggtatccgtgcctactgcctcggagaattccagcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatgtgcttcgttcagatgctttgcttcctgattgtactcagaataccatggcagctcgggctgaggctggttttgaactcttaatactcctgtccgggagtagtagcatctcaggtgtgtccaatcacactctatgtattccttgtttgtgatgtctgtatcactcacccgtgcccttcaaagctttgctaagatgttgcctgggcaatgaggaccttctctttttgctttatttttctgtagccccccacctccccgttttttgggtggttttggtagggatgagacattaagatgttgcctgggcaatgaggaccttctctttttgctttatttttctgtagccccccacctccccgttttttgggtggttttggtagggatgagacaacagggccttatgcaggttatgcaagtaccctattgctgatccacatcccagcctttctggtattttccaccattgaacctgtcatgtggtttgtgtatttcctcagttcatagtcttcttccataaaaacataaatgtggccggacgtggtggcgcatgcctttaatcccagcactcgggaggcagaggcaggtggatttctgagttcgaggccagcctggtctacaaagtgagttccaggacagccagggctatagagaaaccctgtctcaaaaccaaaaaaaaaaaaaaaaaaaaaaaaaaaccaaaaccataaatgtgaggaggtcagggtgttctgtgtttgttggttcttgagctgtacttcaggccgtaacaagttcttagacctcatctgttgaatgagtgaacggaggagtgagaggtccagaaggtaaatatggcgctgtacagttctttttagaagctgtgctgtgggttttgttttttctaagataaaatggggtccagtgacatggctcagagtaaatgtaaatgttgtaccttttggcatcctgggttttatttttccagaacccatttatcctgtgacttccagatgcgcaccataccccctgtaaacccccccccccccaacacacacacacaccaaactagcaaacagatacagggctgtcagcgtgtgagcacatgaggaagggatgctatgttctcaggccctgtgtttcttcttggcaccccctggtttctctgagccctgcacagctacagtgtaaccttacctaattgtcttgttacaaagaagtttgctgcccagagtctaaaggtcagatgagtcacttgggcctcacacttctgggtgtatggaagcaccagatggaagtctcaaggtacacccgacaccggctgcctttccgtcgtcaggcagcacattcctgaagcaggctctctcagctgtgccctctcaaccttcatcctgcagcccattaattgttcaaggccttctagttcttggcaggtctgagcatcttggaaggagctagaagagagatgtagaaagctcaaagacaaaaatgcaaagctacccccatcagataccagctcttgggtctattgaccagccaatatccattgatagtggagttctacatccatatggaagatacaccattctctttcatggacaggagtgccaaatcagaatacaggacattacatctttttgagacaaggtcttaccatgtagcctttgtagaccagctatgtagaccatgctgccctggaacccacagagatttgctagcttctgcctcccaagtgctaagattagtgacacacacctccacttacacagtacacacccaggaaaaaagtaaaacataaataaaaagcaaaaaattgttgtgaaatagctcagtattagagtccttgtctaagatatatgggctccaggcttgcccatccatctattttctgagactggatttttctgtgtagccctggctgtcctgggtcttactttgtagaccaggctgaccttgaactcagagatccacctgcctctgcctctggagctctggggttaaaggtagaccccaccagccatgggcttaagcccagtatgggtttaaaaacatggaaccgattggactttgcataaagttagattggatcctgtgggtgctgacctcccatgctttcaagcacttattggtagcattcactgaacaagcctgagggcgggaacagccctggcatctttcaaagggtatccggttcaaaccagaatcgaatgctcttttcctagccaccccacctcttggcttgttgcctaaggaaaatggctctagcctggttctccagtgttctcatggcagtctggagagattgtccatttgtatcacaaccttttgggcgctgcccttggtgacccgctgctgatgtggacaccagcaagacttgagactgcagacctcctcccccacagatttctcacaatgcccgtgtgggagtgtttgaaaagggaacaagctgaccaacctgtttgtgggaaagtcgtggctttcctatcagaaaacgtcacactctgagtcaggagtgggacatacctgggtactttaacaggtggggcagggtcctgagtcatagagaggtctcacctgggaaggtttctctttcaaagagtgaatcaaccatgacatcacccatctggatctacaccctttcaagccgtctctctccttagcaaacaggaatctaaaagaggaataataaggtgaaagcaaactggaaataggacagaacagacaagggaaaaagttaaagagaaagcttaagagacgcagacacagacgtttacatgcaaacagggggtccataaaaacaaaaccggaagccgtaatatatgtacatggcctgtaagattagaaacaaatgtcctggcacaacattatgggacaacttcccaagctgctgttgagtttgttttgtgttggccggcatgggcccttaaactgagtggtgtgtttctctcttagagaatttttcatgaactagtttttcatttgcaagtggttagtagtttgagatagcttctaggttaggggtggggccgtgtgtccactttactcagctctaatgtcccccgtttggtgcagacccctgcagaccctatgcgtcctgccacagtctctgagttcatgcctgcattgttcaaattgtgtttccttgatgtcctccaatctcctctttgctgggctccctggggcccccaaggtgagggatttggcagaggtattcattccatttagggttgattgttccaaggtccctcactgtacgtgttatttgtcccttctccctggcttcctgctcaggagttctgcagacatggctggtgtttagtagcttcttggtgaagttcaggtagccagtttcatgaccacccagctctcaggcctgactaagcccgtggagaaagcccataaaataaagggggtgaacttctaatctacatctgaaaggagaacccctgtctacatctacctgttaccctagccatcggggagactgaggcagaaggatctagcctggaaatttagggtgagtctgggcagcattaggagactatttcacagtgaagaaggaaggaactcactcacacccttggtgcacctcacaggtctctgggctcagattggagtctttggttttgaaatttcctcatcatcggccgcctcagctggtttttcttctggagtctgttacgccttctcccctttccccccttctcttgctaatcctttcgcttgaacaataggagtccttaaaacctgggagggaagtaaatacctttcagatgagaccctgtcttaagagggggaaaaaaaaaacaaaaaaaccctagggaccgggtcaccaagtgtcaggttaatcagcagatgtcaagatgactcctgagtgcttgctctttcttcagctggtgaaattacagcggcaacatagaatgacagtcagcagggagggaggagagggcctgggtggccaaataaacttataaattgtgagagaaattaatgaatgtctaagttaatgcagaaacggagagacatactatattcatgaactaaaagacttaatattgtgaaggtatactttctttccacataaatttgtagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagccattttcatatttttcttgaacattaagtacaagtctataattaaaaattttttaaatgtagtctggaacattgccagaaacagaagtacaacagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaaagagattgtagaaagttgatacaatttttttttctttaaatatgtcgacgcactggctttacaggggccgtctgctttgtctgtagatatggtgataggagtctgatttagaaacttccttgttctcctcttcccgaatccttcctcctgaacaataggaatacctaagtgcacaagtgcaggtgcccaggaggccctgagacaaccccccctccccagctgcgagccttctccggtggtgctgggaatcgaactggagtcctctgcaagaacagtatgtgctcttaaccactgcaccatctcttcagccccctctagttctttagtttgtacataaattagcaagttcagggaatgcagaagaactggccaggggtcagagccagactggaaacatttttcaccaaacaatagctacagtgttcatggcgtgcctccattgaatactaaactagctgttgatgtgtgactttgatttttaatctttgcaccaactttagttactatatagataattccatttccaaatgatatatagtggaagcttgtaaaagctgcttactatcccagggtccccagccgagatctacatgataaagctactctcaacccagcctacattccccctccctgtgctcagccctacctggggttgttcactttcaagctcccaggaaggaacctgagcgctccctgcccccatgcagtccatttctatctctggtcctttgggtacttggagcctcaagctcctgactgctaatgaagcctgtaacagaagggcattacttactgcccgccttgtgctcccttgaaatctggtcacctggaggtggaagtgggcagtgcttccgtgcttccgtgcttccgctttctcccagctccttttaatcactattttttttttttcttttccaaattactacctaggagttttctagtaatcactttggtgggagtgggagggggagtggaggcagttaactctgtgagtgccaaggacagttgttggacggtggtagtgatggaggggagggtgttcacggtgagggaactctgtggagttacgtagctggcaacaatggggagagacggagccgcaccaccacagacgctttgcatgagcaactattttgttttctgtagatgttttccaaaccttaaactctgttaagtctgaaactgggaggggaacctctccaccaaaatctaaatatggatcagagataaggaaacttccatagtctccagctccctccctgactcagtggcctctgtgaccaactccacgtgactctagtggacaccttcctggtggtttggctttttttttttttccccatttcatcttttaaatggagttttccatgaataagaatataaaactgcgggggctggtgagatggatcagcaggtaagagcacagaggtcctgagttcaaatcccagcaaccacttggtggctcaccatcggtaacgagatctgactccctcttctagagtgtctgaagatagctacagtgtacttacatataataaataaacaaatcttaaaaaaaagaatataaaactgcttccttcccaacaggccttggatagttttgtctgtgggaacagcttcccataggaaaggtgcttttgctacttccgaccgtcacacactcctcctctgcaccccgtgagggcctcccaggagttttgtgttggtcacaagcctccccatgtcccaagggggtctggagagggatctgaggtggtggaaagtgctggggtgccaagttatcagacaatgggcacattgagaatgcaggtggggccaggcctgtgtgggctggagcagctgtcggagcctcagggggtgcagctcatgcgcagagggaagaggcagtctctctgctggctgagaggggagggccacagtaagcaaaggaggggggtgatccccccctcacaagattccagagcaaagggatgtttgaacgatgactcacatgacaggggcggggtctgtgagcaaacttcctgttggaagcttgttcttggttcctgactgtccttcccaggtcatagagactgtctgcaatagggctggggcctgttaggatgccatcgaggtgggaaatcctggagagcttggagtagccagcaggccctcttccctctcctgccccaggctagctggaaacccgcgaggaggaagggtgtgttgttccctatcccatggagaggcttgcatggtcagaccaggcctggtgtgacctctggggctgctctgtgactgccctgttctgtgactgcccttctccctcccagccctccccttccatttgcttctagattcgtctaggagcccatctcagcaaatggcagcatcctcgcttaccagacatcttgtttgtttgtttgtttgttcattcattctgggcctgaagatggagagctgtggtaggggagagctcagaggctgggggcagccttaagggcacagccaggacggagcctctgagcaaagggcagtgtgtgtagagtcaggtaggtggtgggcagcaggagacaggaggagcacagaggtaggtggcactgtacagaggccctggcaccattttgtgacaaggactctcagacccctctagcctattcccccacccccacccccattacaagcagtagggtcttaatagctaatggcttggacaccagcctctggctgctctggttcccagctggtcagagcccttgttctcctttgggttctgatccaggtggcacagggtgggggccctgattcacattcttgatagtgccgctaggttccctgctaccagctggcacattctgtcttcctttccagcaggtacttggctgggcttcctgcccagctgctagacctactgttaggtcagctcattggatgcttcctccaggaagccttctctaatctccattcccttggcttcttttcaagagcagaagttcactggcttctcctgtttccccagagtagcacctggtgtctcaagtactcgctaagcagttaggagaggtgtgaactgggcctcagggaccccagagacaggaggggaggtcattacaggaaagtcggattcttttcctggctttgttcttataaccatcgtgtgtgtgtgtgtgtgtgtgtgtgtgtgtgtgtgtgtgtgtgtgtgtgtgtacgtgtgtgtacgtgtgtgtgtgtgtacatgcgtgcgcgccctacgtcacccgccccgttcccacgccccgcgccacgtcacaaactccaccccctcattatcatattggcttcaatccaaaataaggtatattattgatgatgttacatcg

SEQ ID NO: 79 - DNA, 46 bp, Artificial Sequence
  pGLAd3_5139Acc_CMV_AF_F
attttaaagggccacggtacgacattgattattgactagttattaa

SEQ ID NO: 80 - DNA, 46 bp, Artificial Sequence
  pGLAd3_5139Acc_BGH_AF_R
tgtgtccatccgtgtggtacccatagagcccaccgcatccccagca

SEQ ID NO: 81 - DNA, 46 bp, Artificial Sequence
  pGLAd3_7189Pci_BGH_AF_R
ttaaccccactccccacatggacattgattattgactagttattaa

SEQ ID NO: 82 - DNA, 46 bp, Artificial Sequence
  pGLAd3_7189Pci_BGH_AF_R
gcatctgaacgaagcacatgccatagagcccaccgcatccccagca

(19) United States
(12) Patent Application Publication
(10) Pub. No.: US 2023/0167460 A1
(43) Pub. Date: Jun. 1, 2023

(54) HELPER PLASMID-BASED GUTLESS ADENOVIRUS PRODUCTION SYSTEM

(71) Applicant: GENENMED CO., LTD., Seoul (KR)

(72) Inventor: Dongwoo LEE, Seoul (KR)

(21) Appl. No.: 17/766,646

(22) PCT Filed: Oct. 23, 2020
(86) PCT No.: PCT/KR2020/014585

§ 371 Date: Apr. 5, 2022

Related U.S. Application Data
(60) Provisional application No. 62/924,711, filed on Oct. 23, 2019.

(51) Int. Cl. C12N 15/86

(52) U.S. Cl. CPC C12N 15/86; C12N 2710/10022; C12N 2710/10343; C12N 2710/10352; C12N 2800/50

(57) ABSTRACT

The present invention relates to a helper plasmid-based gutless adenovirus (GLAd) production system, a gutless adenovirus production method using same, a gutless adenovirus produced using same, and a use of gutless adenovirus produced using same.

### TECHNICAL FIELD

The present disclosure relates to a helper plasmid-based gutless adenovirus (GLAd) production system, a gutless adenovirus production method using same, a gutless adenovirus produced using same, and a use of gutless adenovirus produced using same.

### BACKGROUND ART

Gene therapy is emerging as an effective treatment option for various inherited genetic diseases. Gutless adenovirus (hereinafter referred to as “GLAd”), also known as helper-dependent adenovirus (hereinafter referred to “HDAd”), has many notable characteristics as a gene delivery vector for gene therapy, including broad tropism, high infectivity, a large transgene cargo capacity, and an absence of integration into the host genome. Additionally, GLAd ensures long-term transgene expression in host organisms due to its minimal immunogenicity, since it was constructed following the deletion of all the genes from an adenovirus.

However, unavoidable contamination of the highly immunogenic adenovirus used as a helper for GLAd production has remained as a problem that hampers the clinical use of GLAd for the treatment of inherited genetic diseases.

Gene therapy that recovers the normal function of a target gene by replacing a corresponding defective gene is emerging as an effective therapeutic option for various inherited genetic diseases, including Leber's congenital amaurosis (LCA) and spinal muscular atrophy (SMA). This particular type of gene therapy depends upon a vehicle referred to as a vector for delivery of a gene of normal functionality to target tissues or organs.

In gene therapy for the treatment of inherited genetic diseases, two types of gene delivery, that is, ex-vivo gene delivery and in-vivo gene delivery can be considered. Ex vivo gene delivery is a cell therapy product-based approach that utilizes genetically engineered cells. In this approach, vector safety can be continuously monitored at different stages prior to the implantation of genetically manipulated cells into the patient's body. In contrast, in-vivo gene delivery directly transfers a therapeutic transgene to patient tissues or organs as the final destined location. Thus, nothing is more important than vector safety in this approach. In addition to being safe, in-vivo gene delivery vectors should ensure long-term transgene expression to sustain the therapeutic efficacy of the delivered transgene for a long period of time.

Currently, the most commonly used in-vivo gene delivery vector for the clinical treatment of inherited genetic diseases is adeno-associated virus (AAV). The safety of AAV has been well established through a wide variety of clinical trials. AAV exhibits broad tropism for infection and ensures long-term transgene expression in various tissues and organs. These characteristics have drawn considerable attention toward AAV as an in-vivo gene delivery vector for various inherited genetic diseases. Nevertheless, AAV has two notable drawbacks: potential for insertional mutagenesis and a low packaging capacity for the transgene. AAV mainly remains as an episome, but can randomly integrate into the host genome although at a low frequency. This characteristic raises concern about insertional mutagenesis. AAV also possesses a small transgene cargo capacity (˜4.5 kb) and can neither deliver large genes such as huntingtin (9.4 kb) or dystrophin (11 kb), nor can multiple genes be delivered at once by one virus entity. These characteristics of AAV indicates that an ideal in-vivo gene delivery vector must be of high safety profile and large transgene cargo capability, and inability to randomly integrated into a host genome, suggesting that given, the ideal vector could provide better opportunities for in-vivo gene therapy.

GLAd has been considered as a last-generation adenovirus. GLAd is constructed following the deletion of all the genes from an adenovirus, with the resultant expression of no adenoviral proteins. This structural characteristic minimizes the host immune response and allows long-term transgene expression in host tissues or organs. GLAd also shows broad tropism for infection and a high transduction efficiency in transgene delivery. In fact, GLAd is highly comparable to AAV in terms of many safety issues. Moreover, GLAd presents prominent advantages over AAV in regard to genome integration and transgene cargo capacity. GLAd does not integrate into the host genome, which eliminates concern about insertional mutagenesis. GLAd also exhibits a high accommodation capacity (up to 36 kb) for transgenes, hence making it possible to deliver large genes and multiple genes.

However, despite these evident beneficial features, there is a problem associated with the production of the currently available GLAd. Since GLAd is devoid of all adenoviral genes, the production of recombinant GLAd is absolutely dependent upon a helper adenovirus that provides all viral proteins for GLAd packaging. In the standard production process, the helper adenovirus actively replicates while providing helper function and thus remains as a contaminant in the final GLAd preparation. Although a significant reduction of contaminant helper adenovirus can be achieved through Cre-loxP-based excision of the Ψ packaging signal, it is very difficult to completely remove the contaminant helper adenovirus in GLAd production. Moreover, the helper adenovirus can generate a replication-competent adenovirus (RCA) through homologous recombination between helper adenovirus and the E1 region present in packaging cells. These undesirable contaminant helper adenovirus and RCA can cause serious acute and chronic toxicity in host organisms. Furthermore, the host immune response against viral proteins expressed from these contaminant viruses can kill the cells co-infected with recombinant GLAd, and these contaminant viruses, which could result in insufficient expression of GLAd-mediated therapeutic transgenes. These unavoidable problems have raised safety concerns and hindered the clinical use of GLAd despite its unique features and tremendous advantages. Therefore, it is vital to establish a system that can produce recombinant GLAd in the absence of helper adenovirus and thus results in no contamination of helper adenovirus and RCA.

### DISCLOSURE OF INVENTION

### Technical Problem

The present disclosure pertains to a helper plasmid-based gutless adenovirus (GLAd) production system which can product GLAd in the absence of helper adenovirus, a method for production of gutless adenovirus, a gutless adenovirus produced using the same, and a use of the gutless adenovirus produced using the same.

Specifically in the present disclosure, the helper function for GLAd packaging and further amplification is provided by a helper plasmid that does not contain any cis-acting elements required for virus package. Due to the structural characteristic of being free of all cis-acting elements, the helper plasmid is impossible to convert into adenovirus.

Utilizing the helper plasmid, the present disclosure successfully produced large quantities of recombinant GLAd that was completely free of adenovirus and RCA contaminants. The recombinant GLAd that was produced efficiently delivered many target transgenes and exhibited a therapeutic potential for Huntington's disease (HD) and Duchenne muscular dystrophy (DMD). Accordingly, the GLAd production system of the present disclosure is expected to open a new way to the clinical application of GLAd-based gene therapy for various inherited genetic diseases.

Therefore, an aspect of the present disclosure is to provide a helper plasmid-based gutless adenovirus (GLAd) production system.

Another aspect of the present disclosure is to provide a method for production of gutless adenovirus

A further aspect of the present disclosure is to provide gutless adenovirus.

A further another aspect of the present disclosure is to provide a use of gutless adenovirus.

### Solution to Problem

The present disclosure pertains to a helper plasmid-based gutless adenovirus (GLAd) production system, a method for production of gutless adenovirus, a gutless adenovirus produced using the same, and a use of the gutless adenovirus produced using the same.

Below, a detailed description will be given of the present disclosure.

An aspect of the present disclosure is concerned with a gutless adenovirus (GLAd) production system.

The adenovirus genome, which consists of a linear molecule of double-stranded DNA, especially, the genome of adenovirus type 5 is well characterized. For example, there is general conservation associated with positions of E1, IX, E2, IVa2, E3, E4, L1, L2, L3, L4, and L5 genes in the overall structure of adenovirus genome. At each terminus of adenovirus genome is an inverted terminal repeat (ITR) which is necessary for viral replication. Subsequent to the 5′ ITR at the 5′ terminus, the cis-acting element Ψ packaging signal is located which is required for the packaging and encapsidation of adenovirus genome.

The structure of adenovirus genome is described on the basis of the expression order of viral genes after transduction into host cells. More specifically, viral genes are referred to as early (E) or late (L) genes according to whether transcription occurs prior to or after onset of DNA replication. At the early stage of infection, adenovirus expresses E1, E2, E3, and E4 genes in host cells to induce the transduction of host cells for viral replication. The E1 gene, which is considered a master switch, acts as a transcription activator and plays a critical role in both early and later gene transcription; E2 is involved in DNA replication; E3 is responsible for immune modulation; and E4 regulates viral mRNA metabolism.

The basic genome structure of adenovirus is as follows:

5′ inverted terminal repeat (ITR)—Ψ packaging signal—E1—other genes—3′ inverted terminal repeat (ITR).

Here, some elements, for example, 5′ ITR and/or Ψ packaging signal may be somewhere else and then combined later. This recombination can recreate the basic genome structure. Some elements, for example, E1 may be transferred elsewhere and may not return back to the adenovirus genome. That is, the E1 region is delivered to a different cell strain which is then allowed to express the E1 protein and can thus be utilized as an adenovirus production cell strain. Some elements, for example, E3 may be completely eliminated because it does not have any influence on the construction and/or generation of adenovirus.

In the present disclosure, the gutless adenovirus production system may include a helper plasmid, a genome plasmid, and a virus packaging cell strain.

In the present disclosure, the helper plasmid may contain adenovirus genes other than the E1 and E3 regions, but with no limitations thereto. That is, a virus packaging cell strain may include the gene of E1 region, but may not include the gene of E3 region because the absence of the gene does not have any influence on generation of the virus. The helper plasmid supplies adenoviral proteins so that the final genome plasmid carrying a transgene to be expressed and elements necessary for transgene expression is packaged into viruses by using GLAd, that is, GLAdyated.

In the present disclosure, the helper plasmid may include a minimal number of adenovirus genes necessary for GLAd production, except for the adenovirus genes that the virus packaging cell strain expresses, but with no limitations thereto.

Therefore, the helper plasmid according to an embodiment of the present disclosure may have structural characteristics as follows:

None of the 5′ inverted terminal repeat (ITR), the Ψ packaging signal, E1, and the 3′ inverted terminal repeat are contained;

A region between E1 and E3 and a region between E3 and the 3′ inverted terminal repeat are contained (A region stretching from the base immediately after E1 to the base immediately before E3 and a region stretching from the base immediately after E3 to the base immediately before the 3′ inverted terminal repeat) are contained; and

Optionally, E3 may or may not be contained.

As such, the helper plasmid can perform the helper function only, with inability to convert into adenovirus, due to the structural characteristic of being devoid of the 5′ inverted terminal repeat, W packaging signal, E1, and 3′ inverted terminal repeat. Although not including the genes of adenovirus, the helper plasmid free of the cis-acting elements necessary for virus generation cannot convert into adenovirus particles.

In the present disclosure, the helper plasmid may not be an infectious virus particle.

In the present disclosure, the helper plasmid may not convert into an infectious virus particle.

In the present disclosure, the helper plasmid may not include an ITR, but with no limitations thereto.

In the present disclosure, the helper plasmid may not include an ITR and a Ψ packaging signal, but with no limitations thereto.

In the present disclosure, the helper plasmid may further include an antibiotic-resistant gene, for example, a kanamycin-resistant gene, but with no limitations thereto.

In the present disclosure, the helper plasmid may further include an Ori replication origin, but with no limitations thereto.

In the present disclosure, the helper plasmid may be composed of at least one plasmid, for example, one to five plasmids, but with no limitations thereto.

In the present disclosure, the helper plasmid may be pAdBest_dITR, but with no limitations thereto.

In the present disclosure, the genome plasmid may include a 5′ homologous stretch, a 3′ homologous stretch, and a 3′ inverted terminal repeat (ITR).

In the present disclosure, the genome plasmid may further include an antibiotic-resistant gene, for example, a kanamycin-resistant gene, but with no limitations thereto.

In the present disclosure, the genome plasmid may further include an Ori replication origin, but with no limitations thereto.

In the present disclosure, the genome plasma may further include a stuffer DNA (sDNA).

In the present disclosure, the stuffer DNA may further include a scaffold/matrix attachment element (SMAR), but is not limited thereto.

In the present disclosure, the genome plasmid may include a GLAd genome region packaged into the capsid of GLAd, but with no limitations thereto.

In the present disclosure, the genome plasmid may be a final genome plasmid including a transgene gene to be expressed using GLAd, and elements necessary for transgene expression, but with no limitations thereto.

In the present disclosure, the transgene to be expressed using GLAd and elements necessary for transgene expression in the final genome plasmid may be transferred from a cloning shuttle plasmid, but with no limitations thereto.

Therefore, the genome plasmid in the present disclosure may be a final genome plasmid including all cis-acting elements necessary for GLAd production, with or without a transgene and elements necessary for transgene expression being additionally included.

In the present disclosure, the final genome plasmid may be linearized by cutting a region not contained in GLAd genome with a restriction enzyme, but without limitations thereto.

In the present disclosure, the final genome plasmid may include a 5′ inverted terminal repeat (ITR), a Ψ packaging signal, a promoter, an intron, a transgene, a poly(A) signal, a stuffer DNA (sDNA), and a 3′ inverted terminal repeat (ITR).

In the present disclosure, the final genome plasmid may include a GLAd genome region to be packaged into the capsid of GLAd, but with no limitations thereto.

In the present disclosure, the transgene may be at least one gene, but with no limitations thereto.

In the present disclosure, the transgene may have a function of inhibiting expression of a specific gene product, but with no limitations thereto.

In the present disclosure, the transgene may be at least one selected from the group consisting of factor IX (R338L Padua mutant, for treatment of hemophilia B), glucocerebrosidase (GCR, for treatment of Gaucher's disease), hexosaminidase A (HEXA, for treatment of Tay-Sachs disease), hypoxanthine phosphoribosyltransferase 1 (HPRT1, for treatment of Lesch-Nyhan syndrome), iduronate-2-sulfatase (IDS, for treatment of Hunter syndrome), methyl-CpG-binding protein 2 (MECP2, for treatment of Rett syndrome), survival of motor neuron 1 (SMN1, for treatment of spinal muscular atrophy (SMA), and dystrophin gene [for treatment of Duchenne muscular dystrophy (DMD)], but is not limited thereto.

In the present disclosure, the genome plasmid may be pGLAd or pGLAd3, but is not limited thereto.

In the present disclosure, the system may further a cloning shuttle plasmid, but with no limitations thereto.

In the present disclosure, the cloning shuttle plasmid may include a 5′ homologous stretch, a 5′ inverted terminal repeat (ITR), a Ψ packaging signal, a promoter, a multi-cloning site (MCS), a poly(A) signal, and a 3′ homologous stretch, but with no limitations thereto.

In the present disclosure, the promoter may be a CMV promoter or a chicken β-actin promoter, but with no limitations thereto.

In the present disclosure, the cloning shuttle plasmid may include an intron, but with no limitations thereto.

In the present disclosure, the intron may be derived from a rabbit β-globin gene, but with no limitations thereto.

In the present disclosure, the poly(A) signal sequence may be an SV40 poly(A) signal, but with no limitations thereto.

In the present disclosure, the cloning shuttle plasmid may further include an antibiotic-resistant gene, for example, a kanamycin-resistant gene, but with no limitations thereto.

In the present disclosure, the cloning shuttle plasmid may further include an Ori replication origin, but with no limitations thereto.

In the present disclosure, the cloning shuttle plasmid may include a transgene to be expressed using GLAd and elements necessary for transgene expression, but with no limitations thereto.

In the present disclosure, the transgene is at least one gene, but with no limitations thereto.

In the present disclosure, the transgene may have a function of inhibiting expression of a specific gene product, but with no limitations thereto.

In the present disclosure, the transgene may be cloned at a multi-cloning site, but with no limitations thereto.

In the present disclosure, the transgene may be at least one selected from the group consisting of factor IX (R338L Padua mutant, for treatment of hemophilia B), glucocerebrosidase (GCR, for treatment of Gaucher's disease), hexosaminidase A (HEXA, for treatment of Tay-Sachs disease), hypoxanthine phosphoribosyltransferase 1 (HPRT1, for treatment of Lesch-Nyhan syndrome), iduronate-2-sulfatase (IDS, for treatment of Hunter syndrome), methyl-CpG-binding protein 2 (MECP2, for treatment of Rett syndrome), survival of motor neuron 1 (SMN1, for treatment of spinal muscular atrophy (SMA), and dystrophin gene [for treatment of Duchenne muscular dystrophy (DMD)], but is not limited thereto.

In the present disclosure, the cloning shuttle plasmid may be pBest or pBest4, but is not limited thereto.

In the present disclosure, the virus packaging cell strain may a strain that express a protein of the E1 region of adenovirus, for example, HEK293 or HEK293T, but with no limitations thereto.

In the present disclosure, the system may further include a pAd5pTP expression plasmid, but with no limitations thereto. Given, the pAd5pTP expression plasmid may have an effect of remarkably increasing the production of GLAd.

In the present disclosure, the pAd5pTP expression plasmid may include the nucleotide sequence of SEQ ID NO: 76.

Another aspect of the present disclosure pertains to a method for production of gutless adenovirus (GLAd), the method comprising the steps of:

transfecting a final genome plasmid into a virus packaging cell strain; and

transfecting a helper plasmid into the virus packaging cell strain.

Here, the final genome plasmid of the method is as defined above, and thus the description thereof is omitted.

In the present disclosure, the final genome plasmid may be linearized with a restriction enzyme, but with no limitations thereto.

Here, the helper plasmid of the method is as defined above, and thus the description thereof is omitted.

In the present disclosure, the transfection may be carried out by a calcium phosphate precipitation method, but with no limitations thereto.

In the present disclosure, the production method may further comprise a step of transfecting a pAd5pTP expression plasmid into a virus packaging cell strain. Given, this step may lead to a remarkable increase in GLAd production.

In the present disclosure, the pAd5pTP expression plasmid may include the nucleotide sequence of SEQ ID NO: 76.

In the present disclosure, a step of transfecting a final genome plasmid into a virus packaging cell strain and a step of transfecting a helper plasmid into the virus packaging cell strain may be conducted irrespective of the order thereof and, for example, may be conducted simultaneously, but with no limitations thereto.

In the present disclosure, the GLAd produced by the production method may be free of contaminant virus species.

In the present disclosure, the contaminant virus species may be adenovirus or replication-competent adenovirus (RCA), but is not limited thereto.

In an embodiment of the present disclosure, the production method of the present disclosure may be conducted as follows:

A transgene expression cassette in the cloning shuttle plasmid including a transgene is transferred and integrated into a genome plasmid to form a final genome plasmid. The final genome plasmid is linearized with a restriction enzyme. Then, the linearized final genome plasmid and a helper plasmid are transfected into a virus packaging cell strain. GLAd is produced through the transfection step.

In another embodiment of the present disclosure, the production method of the present disclosure may be conducted as follows:

A transgene expression cassette in the cloning shuttle plasmid including a transgene is transferred and integrated into a genome plasmid to form a final genome plasmid. The final genome plasmid is linearized with a restriction enzyme. Then, the linearized final genome plasmid, a helper plasmid, and a pAd5pTP expression plasmid are transfected into a packaging cell strain. Through the transfection step, GLAd is produced.

In a further embodiment of the present disclosure, the production method of the present disclosure may be conducted as follows:

A helper plasmid is transfected into a packaging cell strain. After an adequate period of time, GLAd including a transgene expression cassette is additionally transfected into the transfected cell strain. GLAd is produced after an adequate time. In this regard, the GLAd may be produced in an amplification pattern.

In another embodiment of the present disclosure, the production method of the present disclosure may be conducted as follows:

A helper plasmid and a pAd5pTP expression plasmid are transfected into a virus packaging cell strain. After an adequate period of time, GLAd including a transgene expression cassette is additionally transfected into the transfected cell strain. After an adequate period of time, GLAd is produced. In this regard, the production of GLAd may be implemented in an amplified pattern.

Another aspect of the present disclosure pertains to a method for construction of a cloning shuttle plasmid, the method comprising the steps of:

synthesizing a first DNA fragment corresponding to a Kan^{r}˜ColE1 region in the presence of a primer set, with a pGT2 plasmid serving as a template;

digesting the first DNA fragment with a restriction enzyme;

preparing a second DNA fragment including a 5′ homologous stretch, a 5′ inverted terminal repeat (ITR), a Ψ5 packaging signal, a CMV promoter, a multi-cloning site (MCS), an SV40 poly(A) signal, and a 3′ homologous stretch;

digesting the second DNA fragment with a restriction enzyme; and

ligating the restriction digest of the first DNA fragment to the restriction digest of the second DNA fragment to construct pBest.

In the present disclosure, the pGT2 plasmid may include the nucleotide sequence of SEQ ID NO: 63 and may be, for example, composed of the nucleotide sequence of SEQ ID NO: 63, but is not limited thereto.

In the present disclosure, the primer set in the step of synthesizing a first DNA fragment may be a primer set consisting of the nucleotide sequences of SEQ ID NOS: 1 and 2, but is not limited thereto.

In the present disclosure, the step of synthesizing a first DNA fragment may be carried out by PCR, but with no limitations thereto.

In the present disclosure, the first DNA fragment may contain a restriction site, for example, SfiI and XcmI restriction sites, but with no limitations thereto.

In the present disclosure, the SfiI restriction site may be located at the 3′ end in the first DNA fragment, but with no limitations thereto.

In the present disclosure, the XcmI restriction site may be located at the 5′ end in the first DNA fragment, but with no limitations thereto.

In the present disclosure, the step of digesting the first DNA fragment with a restriction enzyme may be carried out using XcmI and SfiI restriction enzymes, with no limitations thereto.

In the present disclosure, the second DNA fragment may contain a restriction site, for example, SfiI and XcmI restriction sites, with no limitations thereto.

In the present disclosure, the SfiI restriction site may be located at the 5′ end in the second DNA fragment, but with no limitations thereto.

In the present disclosure, the XcmI restriction site may be located at the 3′ end in the second DNA fragment, but with no limitations thereto.

In the present disclosure, the step of digesting the second DNA fragment may be carried out using XcmI and SfiI restriction enzymes, but with no limitations thereto.

In the present disclosure, the step of constructing pBest may be carried out by ligating the restriction digest of the first DNA fragment to the restriction digest of the second DNA fragment, but with no limitations thereto.

In the present disclosure, the pBest may include the nucleotide sequence of SEQ ID NO: 64 and may be, for example, composed of the nucleotide sequence of SEQ ID NO: 64, but is not limited thereto.

Another aspect of the present disclosure pertains to a method for construction of a helper plasmid, the method comprising the following steps.

1. Construction of Ψ5-Left-Arm-2

synthesizing a Kan^{r}˜ColE1 region of Ψ5-Left-Arm-1 in the presence of a primer set, with pGT2 plasmid serving as a template;

digesting the Kan^{r}˜ColE1 region of Ψ5-Left-Arm-1 with a restriction enzyme;

preparing a third DNA fragment corresponding to a BstZ17I˜BamHI region of the Ψ5 genome;

ligating the restriction digest of the third DNA fragment to the restriction digest of the Kan^{r}˜ColE1 region of Ψ5-Left-Arm-1 to prepare Ψ5-Left-Arm-1;

digesting the Ψ5-Left-Arm-1 with a restriction enzyme;

synthesizing a fourth DNA fragment in the presence of a primer set, with Ψ5 serving as a template;

digesting the fourth DNA fragment with a restriction enzyme; and

ligating the restriction digest of the fourth DNA fragment to the restriction digest of the Ψ5-Left-Arm-1 to construct Ψ5-Left-Arm-2.

In the present disclosure, the primer set in the step of synthesizing a Kan^{r}˜ColE1 region of Ψ5-Left-Arm-1 may be a primer set including the nucleotide sequence of SEQ ID NOS: 3 and 4, but with no limitations thereto.

In the present disclosure, the step of synthesizing a Kan^{r}˜ColE1 region of Ψ5-Left-Arm-1 may be carried out by PCR, but with no limitations thereto.

In the present disclosure, the Kan^{r}˜ColE1 region of Ψ5-Left-Arm-1 may contain a restriction site, for example, BamHI and BstZ17I restriction sites, but with no limitations thereto.

In the present disclosure, the BamHI restriction site may be located at the 5′ end in the Kan^{r}˜ColE1 region of Ψ5-Left-Arm-1, but with no limitations thereto.

In the present disclosure, the BstZ17I restriction site may be located at the 3′ end in the Kan^{r}˜ColE1 region of Ψ5-Left-Arm-1, but with no limitations thereto.

In the present disclosure, the step of digesting the Kan^{r}˜ColE1 region of Ψ5-Left-Arm-1 with a restriction enzyme may be carried out using BamHI and BstZ17I restriction enzymes, with no limitations thereto.

In the present disclosure, the third DNA fragment corresponding to the BstZ17I˜BamHI region of the Ψ5 genome may include the nucleotide sequence of SEQ ID NO: 65 and may be, for example, composed of the nucleotide sequence of SEQ ID NO: 65, but is not limited thereto.

In the present disclosure, the third DNA fragment may contain a restriction site, for example, BamHI and BstZ17I restriction sites, but with no limitations thereto.

In the present disclosure, the BamHI restriction site may be located at the 3′ end in the third DNA fragment, but with no limitations thereto.

In the present disclosure, the BstZ17I restriction site may be located at the 5′ end in the third DNA fragment, but with no limitations thereto.

In the present disclosure, the step of preparing a third DNA fragment may be carried out using BamHI and BstZ17I restriction enzymes, with no limitations thereto.

In the present disclosure, the step of preparing Ψ5-Left-Arm-1 may be carried out by ligating the restriction digest of the third DNA fragment to the restriction digest of the Kan^{r}˜ColE1 region of Ψ5-Left-Arm-1, but with no limitations thereto.

In the present disclosure, the step of digesting Ψ5-Left-Arm-1 with a restriction enzyme may be carried out using ClaI and BstZ17I restriction enzymes, but with no limitations thereto.

In the present disclosure, the fourth DNA fragment may be a DNA fragment stretching from ‘*’ (asterisk) mark to the BstZ17I restriction site (b panel in FIG. 7).

In the present disclosure, the primer set in the step of synthesizing a fourth DNA fragment may be a primer set including the nucleotide sequences of SEQ ID NOS: 5 and 6, but with no limitations thereto.

In the present disclosure, the step of synthesizing a fourth DNA fragment may be carried out by PCR, but with no limitations thereto.

In the present disclosure, the fourth DNA fragment may include the nucleotide sequence of SEQ ID NO: 66 and may be, for example, composed of the nucleotide sequence of SEQ ID NO: 66, but with no limitations thereto.

In the present disclosure, the fourth DNA fragment may contain a restriction site, for example, ClaI and BstZ17I enzyme sites, but with no limitations thereto.

In the present disclosure, the ClaI restriction site may be located at the 5′ end in the fourth DNA fragment, but with no limitations thereto.

In the present disclosure, the BstZ17I restriction site may be located at the 3′ end in the fourth DNA fragment, but with no limitations thereto.

In the present disclosure, the step of digesting the fourth DNA fragment with a restriction enzyme may be carried out using ClaI and BstZ17I restriction enzymes, with no limitations thereto.

In the present disclosure, the step of constructing Ψ5-Left-Arm-2 may be carried out by ligating the restriction digest of the fourth DNA fragment to the restriction digest of the Ψ5-Left-Arm-1, with no limitations thereto.

As used herein, the term “Ψ5-Left-Arm-2” refers to a portion of Ad5 (adenovirus type 5, GenBank AC 000008) resulting from deletion of 5′ ITR and Ψ packaging signal, specifically nucleotides 1 to 3133.

2. Construction of Ψ5-Right-Arm-2

the steps of:

synthesizing a Kan^{r}˜ColE1 region of Ψ5-Right-Arm-1 in the presence of a primer set, with pGT2 plasmid, Ψ5-Left-Arm-1, or Ψ5-Left-Arm-2serving as a template;

digesting the Kan^{r}˜ColE1 region of Ψ5-Right-Arm-1 with a restriction enzyme;

preparing a fifth DNA fragment corresponding to a BamHI˜3′ ITR region of the Ψ5 genome;

ligating the restriction digest of the fifth DNA fragment to the restriction digest of the Kan^{r}˜ColE1 region of Ψ5-Right-Arm-1 to prepare Ψ5-Right-Arm-1; digesting the Ψ5-Right-Arm-1 with a restriction enzyme;

synthesizing a sixth DNA fragment in the presence of a primer set, with Ψ5 serving as a template;

digesting the sixth DNA fragment with a restriction enzyme; and

ligating the restriction digest of the sixth DNA fragment to the restriction digest of the Ψ5-Right-Arm-1 to construct Ψ5-Right-Arm-2.

In the present disclosure, the primer set in the step of synthesizing a Kan^{r}˜ColE1 region of Ψ5-Right-Arm-1 may be a primer set including the nucleotide sequence of SEQ ID NOS: 7 and 8, but with no limitations thereto.

In the present disclosure, the step of synthesizing a Kan^{r}˜ColE1 region of Ψ5-Right-Arm-1 may be carried out by PCR, but with no limitations thereto.

In the present disclosure, the Kan^{r}˜ColE1 region of Ψ5-Right-Arm-1 may contain a restriction site, for example, Pad and BamHI restriction sites, but with no limitations thereto.

In the present disclosure, the Pad restriction site may be located at the 5′ end in the Kan^{r}˜ColE1 region of Ψ5-Right-Arm-1, but with no limitations thereto.

In the present disclosure, the BamHI restriction site may be located at the 3′ end in the Kan^{r}˜ColE1 region of Ψ5-Right-Arm-1, but with no limitations thereto.

In the present disclosure, the step of digesting the Kan^{r}˜ColE1 region of Ψ5-Right-Arm-1 with a restriction enzyme may be carried out using BamHI restriction enzyme, with no limitations thereto.

In the present disclosure, the fifth DNA fragment corresponding to the BamHI˜3′ITR region of the Ψ5 genome may include the nucleotide sequence of SEQ ID NO: 67 and may be, for example, composed of the nucleotide sequence of SEQ ID NO: 67, but is not limited thereto.

In the present disclosure, the step of preparing a fifth DNA fragment corresponding to a BamHI˜3′ ITR region of the Ψ5 genome may be carried out by digesting Ψ5 DNA with BamHI restriction enzyme, with no limitations thereto.

In the present disclosure, the BamHI restriction site may be located at the 5′ end in the fifth DNA fragment, but with no limitations thereto.

In the present disclosure, the step of preparing Ψ5-Right-Arm-1 may be carried out by ligating the restriction digest of the fifth DNA fragment to the restriction digest of the Kan^{r}˜ColE1 region of Ψ5-Right-Arm-1, but with no limitations thereto.

In the present disclosure, the step of digesting Ψ5-Right-Arm-1 with a restriction enzyme may be carried out using SpeI and NdeI restriction enzymes, but with no limitations thereto.

In the present disclosure, the primer set in the step of synthesizing a sixth DNA fragment in the presence thereof, with Ψ5 serving as a template is a primer set including nucleotides sequences of SEQ ID NOS: 9 to 12, but with no limitations thereto.

In the present disclosure, the step of synthesizing a sixth DNA fragment may be carried out by PCR, but with no limitations thereto.

In the present disclosure, the sixth DNA fragment may include the nucleotide sequence of SEQ ID NO: 68 and may be, for example, composed of the nucleotide sequence of SEQ ID NO: 68, but with no limitations thereto.

In the present disclosure, the sixth DNA fragment may contain a restriction site, for example, SpeI and NdeI restriction sites, but with no limitations thereto.

In the present disclosure, the SpeI restriction site may be located at the 5′ end in the sixth DNA fragment, but with no limitations thereto.

In the present disclosure, the NdeI restriction site may be located at the 3′ end in the sixth DNA fragment, but with no limitations thereto.

In the present disclosure, the step of digesting the sixth DNA fragment with a restriction enzyme may be carried out using SpeI and NdeI restriction enzymes, with no limitations thereto.

In the present disclosure, the step of constructing Ψ5-Right-Arm-2 may be carried out by ligating the restriction digest of the sixth DNA fragment to the restriction enzyme-digested Ψ5-Right-Arm-1, but with no limitations thereto.

As used herein, the term “Ψ5-Right-Arm-2” refers to a portion of Ad5 (adenovirus type 5, GenBank AC 000008) in which the partially remaining E3 region is completely deleted by removing nucleotides 27864 to 31000 of Ad5.

3. Construction of pAdBest

The steps of:

digesting Ψ5-Right-Arm-2 with a restriction enzyme;

digesting Ψ5-Left-Arm-2 with a restriction enzyme to obtain a larger fragment; and

ligating the restriction digest of the Ψ5-Left-Arm-2 to the restriction digest of the Ψ5-Right-Arm-2 to construct pAdBest.

In the present disclosure, the step of digesting Ψ5-Right-Arm-2 with a restriction enzyme may be carried out using ClaI and BamHI restriction enzymes, but with no limitations thereto.

In the present disclosure, the step of digesting Ψ5-Left-Arm-2 with a restriction enzyme to obtain a larger fragment may be carried out using ClaI and BamHI restriction enzymes.

In the present disclosure, the larger fragment DNA obtained by digesting Ψ5-Left-Arm-2 with ClaI and BamHI restriction enzymes may include the nucleotide sequence of SEQ ID NO: 69 and may be, for example, composed of the nucleotide sequence of SEQ ID NO: 69, but with no limitations thereto.

In the present disclosure, the larger fragment obtained by digesting Ψ5-Left-Arm-2 with a restriction enzyme may contain a restriction site, for example, ClaI and BamHI restriction sites, but with no limitations thereto.

In the present disclosure, the ClaI restriction site may be located at the 5′ end in the larger fragment obtained by digesting Ψ5-Left-Arm-2 with a restriction enzyme, but with no limitations thereto.

In the present disclosure, the BamHI restriction site may be located at the 3′ end in the larger fragment obtained by digesting Ψ5-Left-Arm-2 with a restriction enzyme, but with no limitations thereto.

In the present disclosure, the step of constructing pAdBest may be carried out by ligating the restriction digest of the Ψ5-Right Arm-2 with the larger fragment obtained by digesting Ψ5-Left Arm-2 with a restriction enzyme, but with no limitations thereto.

4. Construction of pAdBest_dITR

The steps of:

digesting pAdBest with a restriction enzyme to obtain a smaller fragment;

ligating an adaptor containing ClaI and EcoRI restriction sites to the smaller fragment obtained by digesting pAdBest with a restriction enzyme to prepare pAdBest_EcoR_Cla;

digesting pAdBest_EcoR_Cla with a restriction enzyme to obtain a larger fragment;

synthesizing a seventh DNA fragment in the presence of a primer set, with pAdBest serving as a template;

digesting the seventh DNA fragment with a restriction enzyme;

ligating the restriction digest of the seventh DNA fragment to the larger fragment obtained by digesting pAdBest_EcoR_Cla with a restriction enzyme to prepare pAdBest_EcoR_Cla_dITR;

digesting pAdBest_EcoR_Cla_dITR with a restriction enzyme;

digesting pAdBest with a restriction enzyme to obtain a larger fragment; and

ligating the restriction digest of the pAdBest_EcoR_Cla_dITR with the larger fragment obtained by digesting pAdBest with a restriction enzyme to construct pAdBest_dITR.

In the present disclosure, the step of digesting pAdBest with a restriction enzyme to obtain a smaller fragment may be carried out using ClaI and EcoRI restriction enzymes, but with no limitations thereto.

In the present disclosure, the smaller fragment obtained by digesting pAdBest with a restriction enzyme may contain a restriction site, for example, ClaI and EcoRI restriction site, but with no limitations thereto.

In the present disclosure, the ClaI restriction site may be located at the 3′ end of the smaller fragment obtained by digesting pAdBest with a restriction enzyme, but with no limitations thereto.

In the present disclosure, the EcoRI restriction site may be located at 5′ end of the smaller fragment obtained by digesting pAdBest with a restriction enzyme, but with no limitations thereto.

In the present disclosure, the adaptor containing ClaI and EcoRI restriction sites may be an adaptor including the nucleotide sequences of SEQ ID NOS: 13 and 14, but with no limitations thereto.

In the present disclosure, the step of preparing pAdBest_EcoR_Cla may be carried out by ligating the adaptor to the smaller fragment obtained by digesting pAdBest with a restriction enzyme, but with no limitations thereto.

In the present disclosure, the step of digesting pAdBest_EcoR_Cla with a restriction enzyme to obtain a larger fragment may be carried out using AvrII and RsrII restriction enzymes, but with no limitations thereto.

In the present disclosure, the larger fragment obtained by digesting pAdBest_EcoR_Cla with a restriction enzyme may contain a restriction site, for example, AvrII and RsrII restriction sites, but with no limitations thereto.

In the present disclosure, the AvrII restriction site may be located at the 3′ end of the larger fragment obtained by digesting pAdBest_EcoR_Cla with a restriction enzyme, but with no limitations thereto.

In the present disclosure, the RsrII restriction site may be located at the 5′ end of the larger fragment obtained by digesting pAdBest_EcoR_Cla with a restriction enzyme, but with no limitations thereto.

In the present disclosure, the primer set in the step of synthesizing a seventh DNA fragment may include nucleotide sequences of SEQ ID NOS: 15 to 18, but with no limitations thereto.

In the present disclosure, the step of preparing a seventh DNA fragment may be carried out by overlapping PCR, but with no limitations thereto.

In the present disclosure, the seventh DNA fragment may include the nucleotide sequence of SEQ ID NO: 70, for example, may be composed of the nucleotide sequence of SEQ ID NO: 70, but with no limitations thereto.

In the present disclosure, the seventh DNA fragment may contain a restriction site, for example, AvrII and RsrII restriction site, but with no limitations thereto.

In the present disclosure, the step of digesting the seventh DNA fragment with a restriction site may be carried out using AvrII and RsrII restriction enzymes, but with no limitations thereto.

In the present disclosure, the AVrII restriction site may be located at the 5′ end in the seventh DNA fragment, but with no limitations thereto.

In the present disclosure, the RsrII restriction site may be located at 3′ end in the seventh DNA fragment, but with no limitations thereto.

In the present disclosure, the step of preparing pAdBest_EcoR_Cla_dITR may be carried out by ligating the restriction digest of the seventh DNA fragment to the larger fragment obtained by digesting pAdBest_EcoR_Cla with a restriction enzyme, but with no limitations thereto.

In the present disclosure, the step digesting pAdBest_EcoR_Cla_dITR with a restriction enzyme may be carried out using ClaI and EcoRI restriction enzymes, but with no limitations thereto.

In the present disclosure, the step of digesting pAdBest with a restriction enzyme to obtain a larger fragment may be carried out using ClaI and EcoRI restriction enzymes, but with no limitations thereto.

In the present disclosure, the larger fragment obtained by digesting pAdBest with a restriction enzyme may contain a restriction site, for example, ClaI and EcoRI restriction sites, but with no limitations thereto.

In the present disclosure, the ClaI restriction site may be located at the 5′ end in the larger fragment obtained by digesting pAdBest with a restriction enzyme, but with no limitations thereto.

In the present disclosure, the EcoRI restriction site may be located at the 3′ end in the larger fragment obtained by digesting pAdBest with a restriction enzyme, but with no limitations thereto.

In the present disclosure, the step of constructing pAdBest ITR may be carried out by ligating the restriction digest of the pAdBest_EcoR_Cla_dITR with the larger fragment obtained by digesting pAdBest with a restriction enzyme, but with no limitations thereto.

In the present disclosure, the pAdBest_dITR may include the nucleotide sequence of SEQ ID NO: 71, for example, may be composed of the nucleotide sequence of SEQ ID NO: 71, but with no limitations thereto.

Another aspect of the present disclosure pertains to a method for constructing a genome plasmid, the method comprising the steps of:

synthesizing an eighth DNA fragment in the presence of a primer set, with pAdBest plasmid serving as a template;

digesting the eighth DNA fragment with a restriction enzyme;

ligating a restriction digest of the eighth DNA fragment to an adaptor to prepare pAdBestGL1;

removing a restriction site from pAdBestGL1 to prepare pAdBestGL1_dCla;

digesting pAdBestGL1 and pAdBestGL1_dCla with respective restriction enzymes;

ligating restriction digests of pAdBestGL1 and pAdBestGL1_dCla to adaptors to prepare pAdBestGL2_wtCla and pAdBestGL2, respectively;

digesting pAdBestGL2 with a restriction enzyme;

mixing the restriction digest of pAdBestGL2 and a restriction digest of lambda phage to prepare pAdBestGL3;

digesting pAdBestGL3 with a restriction enzyme;

ligating the restriction digest of pAdBestGL3 to an adaptor to prepare pAdBestGL4_3H;

ligating the restriction digest of pAdBestGL3 to an adaptor to prepare pAdBestGL4_5H;

removing a restriction site from pAdBestGL4_3H to prepare pAdBestGL4_3H_2dCla;

removing a restriction site from pAdBestGL4_5H to prepare pAdBestGL4_5H_dCla;

digesting pAdBestGL4_3H_2dCla with a restriction enzyme;

digesting pAdBestGL4_5H_dCla with a restriction enzyme to obtain a larger fragment;

ligating the restriction digest of pAdBestGL4_3H_2dCla with the larger fragment obtained by digesting pAdBestGL4_5H_dCla with a restriction enzyme to prepare pAdBestGL5;

digesting pAdBestGL2_wtCla with a restriction enzyme;

digesting pAdBestGL5 with a restriction enzyme;

mixing the restriction digest of pAdBestGL2_wtCla with the restriction digest of pAdBestGL5 to prepare pAdBestGL;

digesting pAdBestGL with a restriction enzyme;

preparing a scaffold/matrix attachment (SMAR) element;

digesting the SMAR element with a restriction enzyme; and

ligating the restriction digest of pAdBestGL to the restriction digest of the SMAR element.

In the present disclosure, the primer set in the step of synthesizing an eighth DNA fragment may be a primer set including the nucleotide sequences of SEQ ID NOS: 19 and 20, but with no limitations thereto.

In the present disclosure, the step of synthesizing an eighth DNA fragment may be carried out using PCR, but with no limitations thereto.

In the present disclosure, the eighth DNA fragment may include the nucleotide sequence of SEQ ID NO: 72, for example, may be composed of the nucleotide sequence of SEQ ID NO: 72, but with no limitations thereto.

In the present disclosure, the eighth DNA fragment may contain a restriction site, for example, SacI and AscI restriction sites, but with no limitations thereto.

In the present disclosure, the step of digesting the eighth DNA fragment with a restriction enzyme may be carried out using SacI and AscI restriction enzymes, but with no limitations thereto.

In the present disclosure, the SacI restriction site may be located at the 3′ end in the eighth DNA fragment, but with no limitations thereto.

In the present disclosure, the AscI restriction site may be located at the 5′ end in the eighth DNA fragment, but with no limitations thereto.

In the present disclosure, the adaptor in the step of preparing pAdBestGL1 may include the nucleotide sequences of SEQ ID NOS: 21 and 22, but with no limitations thereto.

In the present disclosure, the step of preparing pAdBestGL1 by ligating the adaptor to the restriction digest of the eighth DNA fragment, but with no limitations thereto.

In the present disclosure, the restriction site in the step of preparing a restriction site from pAdBestGL1 to prepare pAdBestGL1_dCla may be the ClaI restriction site, but is not limited thereto.

In the present disclosure, the step of preparing pAdBestGL1_dCla may be carried out by digesting pAdBestGL1 with ClaI restriction enzyme and filling the gap with Klenow, followed by allowing self-ligation, but with no limitations thereto.

In the present disclosure, the step of digesting pAdBestGL1 and pAdBestGL1_dCla with respective restriction enzymes may be carried out using SacI and AvrII restriction enzymes, but with no limitations thereto.

In the present disclosure, the adaptors in the step of preparing pAdBestGL2_wtCla and pAdBestGL2 may each include the nucleotide sequences of SEQ ID NOS: 23 and 24 (FIG. 8b), but with no limitations thereto.

In the present disclosure, the step of preparing pAdBestGL2_wtCla and pAdBestGL2 may be carried out by ligating the restriction digests of pAdBestGL1 and pAdBestGL1_dCla to the adaptors, respectively, but with no limitations thereto.

In the present disclosure, the step of digesting pAdBestGL2 with a restriction enzyme may be carried out using HpaI restriction enzyme, but with no limitations thereto.

In the present disclosure, the restriction digest of lambda phage DNA may be obtained by digesting lambda phage DNA with HindIII restriction enzyme, but with no limitations thereto.

In the present disclosure, the step of preparing pAdBestGL3 may be carried out by mixing the restriction digest of pAdBestGL2 and the restriction digest of lambda phage DNA and subjecting the mixture to in vitro homologous annealing (iHoA), but with no limitations thereto.

In the present disclosure, the step of digesting pAdBestGL3 with a restriction enzyme may be carried out using SacI and ApaI restriction enzymes or ApaI and

AvrII restriction enzymes, but with no limitations thereto.

In the present disclosure, the restriction digest of pAdBestGL3 in the step of preparing pAdBestGL4_3H may be obtained by digesting pAdBestGL3 with SacI and ApaI restriction enzymes, but with no limitations thereto.

In the present disclosure, the adaptor in the step of preparing pAdBestGL4_3H may be an adaptor including the nucleotides of SEQ ID NOS: 25 and 26 (FIG. 8d), but is not limited thereto.

In the present disclosure, the step of preparing pAdBestGL4_3H may be carried out by ligating the adaptor to the restriction digest of pAdBestGL3, but with no limitations thereto.

In the present disclosure, the restriction digest of pAdBestGL3 in the step of preparing pAdBestGL4_5H may be obtained by digesting pAdBestGL3 with ApaI and AvrII restriction enzymes, but with no limitations thereto.

In the present disclosure, the adaptor in the step of preparing pAdBestGL4_5H may be an adaptor including the nucleotides of SEQ ID NOS: 27 and 28 (FIG. 8e), but with no limitations thereto.

In the present disclosure, the step of preparing pAdBestGL4_5H may be carried out by ligating the adaptor to the restriction digest of pAdBestGL3, but with no limitations thereto.

In the present disclosure, the restriction site to be removed in the step of removing a restriction site from pAdBestGL4_3H to prepare pAdBestGL4_3H_2dCla may be a ClaI restriction site, but is not limited thereto.

In the present disclosure, the step of preparing pAdBestGL4_3H_2dCla may comprise the steps of: digesting pAdBestGL4_3H with ClaI restriction enzyme, filling the gap with Klenow, subjecting the same to self-ligation, and transforming the resultant plasmid into Dam−/− bacterial cells; digesting the resulting pAdBestGL4_3H_dCla with ClaI restriction enzyme, again; filling the gap with Klenow; and subjecting the same to self-ligation.

In the present disclosure, the restriction site to be removed in the step of removing a restriction site from pAdBestGL4_5H to prepare pAdBestGL4_5H_2dCla may be a ClaI restriction site, but is not limited thereto.

In the present disclosure, the step of preparing pAdBestGL4_5H_dCla may be carried out by digesting pAdBestGL4_5H with ClaI restriction enzyme, filling the gap with Klenow, and subjecting the same to self-ligation, but with no limitations thereto.

In the present disclosure, the step of digesting pAdBestGL4_3H_2dCla with a restriction enzyme may be carried out using SacI and ApaI restriction enzymes, but with no limitations thereto.

In the present disclosure, the step of digesting pAdBestGL4_5H_dCla with a restriction enzyme to obtain a larger fragment may be carried out using SacI and ApaI restriction enzymes, but with no limitations thereto.

In the present disclosure, the larger fragment obtained by digesting pAdBestGL4_5H_dCla with a restriction enzyme may contain a restriction site, for example, SacI and ApaI restriction sites, but with no limitations thereto.

In the present disclosure, the SacI restriction site may be located at the 5′ end in the larger fragment obtained by digesting pAdBestGL4_5H_dCla with a restriction enzyme, with no limitations thereto.

In the present disclosure, the ApaI restriction site may be located at the 3′ end in the larger fragment obtained by digesting pAdBestGL4_5H_dCla with a restriction enzyme, but with no limitations thereto.

In the present disclosure, the step of preparing pAdBestGL5 may be carried out by ligating the restriction digest of pAdBestGL4_3H_2dCla with the larger fragment obtained by digesting pAdBestGL4_5H_dCla with a restriction enzyme, but with no limitations thereto.

In the present disclosure, the step of digesting pAdBestGL2_wtCla with a restriction step may be carried out using HpaI restriction enzyme, but with no limitations thereto.

In the present disclosure, the step of digesting pAdBestGL5 with a restriction enzyme may be carried out using SacI and AvrII restriction enzymes, but with no limitations thereto.

In the present disclosure, the step of preparing pAdBestGL may be carried out by mixing the restriction digest of pAdBestGL2_wtCla and the restriction digest of pAdBestGL5 and subjecting the mixture to iHoA, but with no limitations thereto.

In the present disclosure, the step of digesting pAdBestGL with a restriction enzyme may be carried out using ApaI and NsiI restriction enzymes, but with no limitations thereto.

In the present disclosure, the primer set in the step of preparing a scaffold/matrix attachment element may be a primer set including SEQ ID NOS: 29 and 30, but is not limited thereto.

In the present disclosure, the step of preparing a scaffold/matrix attachment element may be carried out PCR, but with no limitations thereto.

In the present disclosure, the scaffold/matrix attachment element may contain a restriction site, for example, ApaI and NsiI restriction sites, but with no limitations thereto.

In the present disclosure, the step of digesting the scaffold/matrix attachment element with a restriction enzyme may be carried out using ApaI and NsiI restriction enzymes, but with no limitations thereto.

In the present disclosure, the ApaI restriction site may be located at the 5′ end in the scaffold/matrix attachment element, but with no limitations thereto.

In the present disclosure, the NsiI restriction site may be located at the 3′ end in the scaffold/matrix attachment element, but with no limitations thereto.

In the present disclosure, the step of constructing pGLAd may be carried out by ligating the restriction digest of the scaffold/matrix element to the restriction digest of pAdBestGL, with no limitations thereto.

In the present disclosure, the pGLAd may include the nucleotide sequence of SEQ ID NO: 73, for example, may be composed of the nucleotide sequence of SEQ ID NO: 73, but with no limitations thereto.

Another aspect of the present disclosure pertains to a gutless adenovirus including the following composition:

5′ inverted terminal repeat (ITR), a Ψ packaging signal, a promoter, an intron, a transgene, a poly(A) signal, a stuffer DNA (sDNA), and 3′ inverted terminal repeat.

In the present disclosure, the promoter is a CMV promoter or a chicken β-actin promoter, but with no limitations thereto.

In the present disclosure, the intron may be derived from a rabbit β-globin gene, but with no limitations thereto.

In the present disclosure, the poly(A) signal may be an SV40 poly(A) signal, but with no limitations thereto.

In the present disclosure, the stuffer DNA may further include a scaffold/matrix attachment element (SMAR), but with no limitations thereto.

In the present disclosure, the transgene gene may be at least one gene, but with no limitations thereto. In the present disclosure, the transgene may have a function of inhibiting expression of a specific gene, but with no limitations thereto.

In the present disclosure, the transgene may be at least one selected from the group consisting of factor IX (R338L Padua mutant, for treatment of hemophilia B), glucocerebrosidase (GCR, for treatment of Gaucher's disease), hexosaminidase A (HEXA, for treatment of Tay-Sachs disease), hypoxanthine phosphoribosyltransferase 1 (HPRT1, for treatment of Lesch-Nyhan syndrome), iduronate-2-sulfatase (IDS, for treatment of Hunter syndrome), methyl-CpG-binding protein 2 (MECP2, for treatment of Rett syndrome), survival of motor neuron 1 (SMN1, for treatment of spinal muscular atrophy (SMA), and dystrophin gene [for treatment of Duchenne muscular dystrophy (DMD)], but is not limited thereto.

In the present disclosure, the gutless adenovirus may be produced using the helper plasmid-based gutless adenovirus production system described above, but with no limitations thereto.

In the present disclosure, the gutless adenovirus may be produced by the gutless adenovirus production method described above, but with no limitations thereto.

### Advantageous Effects of Invention

The present disclosure pertains to a helper plasmid-based gutless adenovirus (GLAd) production system, a method for producing a gutless adenovirus, using same, a gutless adenovirus produced using same, and a use of the gutless adenovirus produced using same. The helper plasmid-based GLAd production system of the present disclosure can produce only the recombinant GLAd, without generating helper plasmid-derived adenovirus and replication-competent adenovirus (RCA). The recombinant GLAd produced by the production system can effectively deliver genes irrespective of sizes of the transgenes, exhibiting a therapeutic potential for Huntington's disease (HD) and Duchenne muscular dystrophy (DMD). Accordingly, the helper plasma-based GLAd production system of the present disclosure is suggested to be a new flatform for the clinical application of GLAd-based gene therapy for various inherited genetic diseases.

### BRIEF DESCRIPTION OF DRAWINGS

FIG. 1 shows construction of the pAdBest_dITR helper plasmid and the pGLAd genome plasmid and their use for producing GLAd in accordance with an embodiment of the present disclosure:

a. Structural characteristics of the most commonly used helper adenovirus. The black arrowheads indicate loxP sites;

b. Ad5 genome structure and transcription units. E and L indicate the early gene and the late gene, respectively. The arrows indicate the orientation of transcription units;

c. Schematic illustration and comparison of Ad5, pAdBest_dITR helper plasmid, pGLAd genome plasmid, and GLAd genome structures. SMAR indicates the scaffold/matrix attachment element. Colors indicate the origin of the DNA backbones;

d. Schematic illustration of GLAd production. Co-transfection of the pAdBest_dITR helper plasmid and the PacI-linearized recombinant pGLAd_X genome plasmid into HEK293T cells produces recombinant GLAd.X virus; and

e. LacZ staining of HEK293 cells infected with the GLAd.LacZ virus. At 48 hours after the infection of HEK293 cells with the GLAd.LacZ virus, the cells were subjected to LacZ staining. Upper: untreated control (background dots are X-gal crystals, not stained cells); middle: 50 μl out of 10 ml viral medium was used for infection; bottom: 5 μl out of 1 ml viral lysate was used for infection

FIG. 2 shows construction of the pGLAd3, which is a novelgenome plasmid, and its use for producing GLAd in accordance with an embodiment of the present disclosure:

a. Schematic illustration and comparison of pGLAd and pGLAd3 genome plasmid and corresponding GLAd. Colors show the origin of the DNA backbones;

b. Cytopathic effect (CPE) observed during the production of recombinant GLAd3.LacZ virus. The pAdBest_dITR helper plasmid and the PacI-cut pGLAd3_LacZ genome plasmid were co-transfected into HEK293T cells. Forty-eight hours later, untreated control (left) and transfected cells (right) were photographed under the microscope;

c. LacZ staining of HEK293 cells infected with the GLAd3.LacZ virus. At 48 h after the infection of HEK293 cells with the GLAd3.LacZ virus, the cells were subjected to LacZ staining. The numbers indicate the viral lysate volume (0, 5, or 20 μl) used for infection from 1 ml viral lysate prepared from a 100 mm culture dish; and

d. Homologous regions identified in the helper adenovirus, E1-expressing packaging cells and our pAdBest_dITR helper plasmid. Homologous recombination can occur in colored boxed regions. RCA generation requires two homologous recombination events, through which E1 is transferred from the packaging cell to the helper adenovirus

FIG. 3 shows workflow for large-scale GLAd production in accordance with an embodiment of the present disclosure:

a. The entire process of the standard conventional GLAd production method. The pHDAd.LacZ is a GLAd genome plasmid containing the LacZ expression cassette. The titer indicates the total infectious GLAd particles (BFU determined by LacZ staining) produced in each round of amplification; and

b The entire process for seed GLAd (P0) rescue, continuous amplification (P1 to P3) and large-scale GLAd production (P3, P4, or P5, depending on scale) is described (for details, see Materials and methods). The titer (BFU) was determined by LacZ staining in each round of amplification.

FIG. 4 shows analysis of adenovirus and RCA contaminants generated during GLAd production in accordance with an embodiment of the present disclosure:

a. Serial infection of HEK293 cells in 100 or 150 mm culture dishes with Ad.LacZ (positive control) or P3 GLAd3.LacZ (3×10⁹ BFU) (see FIG. 3 for production), respectively. Under these conditions, replicable adenovirus and RCA can be amplified. In the positive control, one infectious virus particle resulted in 5×10⁵ infectious virus particles (1 BFU to 5×10⁵ BFU) within less than two complete rounds of amplification. Benzonase was used to degrade the helper plasmid continuously used for the preparation of P3 GLAd3.LacZ (viral DNA packaged into the capsid shell is resistant to Benzonase). In each round of amplification, the cells were collected and processed to release adenovirus and RCA. In the final round of amplification, both the cells and culture medium were collected and processed for the analysis of adenovirus and RCA;

b. Analysis of adenovirus and RCA in the samples (a) by PCR. The samples were analyzed by PCR for the N-terminal DNA of the fiber gene, which is present in both the adenovirus and RCA but not in the GLAd genome or HEK293 cells. Ad5 DNA (10 μg) was used as a positive control for PCR. The Ad.LacZ sample was subjected to PCR before or after 100× dilution (in this dilution, only 500 virus particles were present in the sample). The GLAd3.LacZ sample was PCR-amplified without or with spiked Ad5 DNA (10 μg). The arrowhead indicates the target PCR product (484 bp). M is a 100 bp size marker;

c. Infection of HEK293 cells with P4 GLAd3.LacZ or P5 GLAd3.LacZ for the amplification of adenovirus and RCA, as shown in a. The prepared samples were subjected to PCR analysis (d); and

d. Analysis of adenovirus and RCA in samples (c) by PCR. PCR was carried out as described in b.

FIG. 5 shows expression of huntingtin mshRs, the codon-optimized synthetic huntingtin gene, or both by recombinant GLAd in accordance with an embodiment of the present disclosure:

a. Schematic illustration of the full-length human mature huntingtin mRNA and the locations of mshRs. The numbers indicate the locations of the target sites of mshR1, mshR2, and mshR3;

b. Template for mshRs. The 21NTs are sense and anti-sense sequences of 21 nucleotides in length;

c. Expression plasmid for mshRs. Individual mshR (Table S2) was cloned into this plasmid using the BamHI and EcoRI sites;

d. Inhibition of endogenous huntingtin expression by mshR expression plasmids. HEK293T cells were either left untreated or were transfected with pGT2, pGT2-mshR1, pGT2-mshR2, or pGT2-mshR3. Forty-eight hours later, the cells were harvested and subjected to western blotting analysis of endogenous huntingtin expression. HTT denotes huntingtin;

e. Schematic illustration of the full-length human mature huntingtin mRNA and the locations of mshRs. The upper black and lower red horizontal lines indicate the native and codon-optimized huntingtin mRNAs, respectively. The numbers indicate the locations of the target sites of mshR1, mshR2, and mshR3. For mshR1 and mshR3, the amino acids and their corresponding codons are shown. Nucleotides identified as different between the native (endogenous) and codon-optimized huntingtin mRNA are highlighted with gray;

f. pGLAd4 genome plasmid for cloning mshR1, mshR3, and the codon-optimized synthetic huntingtin gene (right orientation or reverse (R) orientation);

g. Recombinant GLAd delivering both huntingtin mshRs and the codon-optimized synthetic hungtingtin gene simultaneously. GLAd4.coHTT.HTTmshR1/3 delivers the right-oriented codon-optimized hungtingtin gene. The total length (28.3 kb) is the size from the 5′ ITR to the 3′ ITR;

h. Effects of mshR1/3 and the codon-optimized synthetic huntingtin gene on huntingtin expression when simultaneously delivered by recombinant GLAd. At 48 h after the infection of HEK293T cells with GLAd4.coHTT.HTTmshR1/3 or GLAd4.coHTT(R). HTTmshR1/3 (reverse oriented huntingtin gene), the cells were harvested and subjected to western blotting for the analysis of huntingtin expression. HTT denotes huntingtin; and

i. Action model of recombinant GLAd4.coHTT.HTTmshR1/3 in HD. wtHTT and mtHTT indicate wild type and mutant huntingtin mRNA, respectively. coHTT denotes mRNA transcribed from the codon-optimized synthetic huntingtin gene. Recombinant GLAd4.coHTT.HTTmshR1/3 simultaneously delivers both mshRs and the codon-optimized synthetic huntingtin gene to target HD tissues.

FIG. 6 shows recombinant GLAd delivers the dystrophin gene in vivo in accordance with an embodiment of the present disclosure in accordance with an embodiment of the present disclosure:

a. Schematic illustration of recombinant GLAd4.Dys. The total length (27.7 kb) is the size from the 5′ ITR to the 3′ ITR;

b. Time frame of animal experiments; and

c. Examination of dystrophin expression in dystrophin-knockout MDX mice. The focal gastrocnemius muscle of MDX mice was injected with PBS or 50 μl of the GLAd4.Dys virus (4×1010 viral particles). Four weeks later, muscle tissues biopsied from the wild-type control and MDX mice treated with PBS or the GLAd4. Dys virus were subjected to immunofluorescence staining and analyzed under a confocal microscope (magnification=×200; scale bar=20 μm).

FIG. 7 shows construction of the pAdBest_dITR helper plasmid and the pBest cloning shuttle plasmid in accordance with an embodiment of the present disclosure;

a. Schematic illustration of Ψ5, a derivative of Ad5. Vertical dotted lines in E1 and E3 indicate actual deletion locations in the corresponding gene. The unique BamHI is shown;

b-e. Construction scheme for the pAdBest_dITR helper plasmid. For details, see Materials and methods; and

f. Structural configuration of the cloning shuttle plasmid pBest. Black and gray boxes indicate the 5′ and 3′ homologous stretches for iHoA.

FIG. 8 shows construction of the pGLAd genome plasmid. For details see Materials and methods.

FIG. 9 shows schematic illustration of iHoA for the construction of recombinant pGLAd_LacZ in accordance with an embodiment of the present disclosure;

a. The black boxes indicate PmeI sites, and arrowheads indicate the cleavage position of PmeI. PmeI-cleaved pBest_LacZ was mixed with ClaI-cut pGLAd and subjected to iHoA. 68 bp and 49 bp indicate the 5′ and 3′ homologous stretches, respectively, between pBest and pGLAd;

b. Intermediate result of iHoA. Aligned double-stranded DNAs (a) were converted from single-stranded ones. This process resulted in hybrid-annealing and transferred the 5′ ITR, Ψ packaging signal and LacZ expression cassette to the pGLAd genome plasmid; and

c. Result of completed iHoA. The annealed strands are repaired in transformed bacterial cells.

FIG. 10 shows efficiency of iHoA in the construction of recombinant pGLAd_X in accordance with an embodiment of the present disclosure;

a. Bacterial colonies formed on an agar plate following iHoA. The arrows indicate smaller colonies, potentially containing the correct recombinant pGLAd_X plasmids;

b. The restriction map for pGLAd_LacZ. E and B indicate EcoRI and BamHI sites, respectively. Numbers denote the locations of the corresponding restriction sites;

c, Colony screening result. Five smaller colonies were picked and subjected to plasmid purification. The plasmids were digested with EcoRI and resolved on an agarose gel. M is the lambda HindIII size marker; and

d, Additional restriction digestion results. Clones 1 and 2 were doubly digested with EcoRI and BamHI. M is the lambda HindIII size marker.

FIG. 11 shows sequence verification of iHoA junctions in accordance with an embodiment of the present disclosure:

a. iHoA result. The colored arrowheads indicate sequencing primers. The arrowhead points to the starting nucleotide of the ClaI site (5′-ATCGAT-3′, destroyed after iHoA) used for the linearization of pGLAd in iHoA. The arrows indicate the ends of the homologous stretches used for iHoA; and

b and c. Sequencing results. The junction points are boxed. The arrows and arrowhead indicate the corresponding position described in a.

FIG. 12 shows construction of the pGLAd3, which is a novelgenome plasmid in accordance with an embodiment of the present disclosure;

a. Schematic illustration of the mouse E-cadherin intron 2 region. The vertical black boxes indicate exons of the E-cadherin gene. The horizontal lines show the PCR products obtained using the primer sets (Table 20). N, C and F1-F5 represent the names of the PCR products. The numbers on the horizontal lines indicate the length of the PCR products.

b. Construction scheme for the pGLAd3. For details, see Materials and methods; and

c. Schematic illustration of the pBest4 cloning shuttle plasmid. Black and gray boxes indicate the 5′ and 3′ homologous stretches for iHoA. MCS represents the multi-cloning sites for transgenes.

FIG. 13 shows two kinds of helpers for GLAd production and their effects on the generation of adenovirus and RCA contaminants in accordance with an embodiment of the present disclosure through schematic representation of GLAd production with helpers in HEK293T or HEK293 packaging cells. Homologous recombination can occur in the colored boxed regions. RCA represents replication-competent adenovirus.

FIG. 14 shows conversion of the pGLAd3 into the pGLAd4 genome plasmid in accordance with an embodiment of the present disclosure. The pGLAd3 contains two BssHII sites. The pGLAd3 was digested with BssHII and self-ligated. This process deleted the portion indicated with the asterisk, decreasing the total length from 26,597 bp to 16,392 bp.

### BEST MODE FOR CARRYING OUT THE INVENTION

The present disclosure pertains to a helper plasmid-based gutless adenovirus (GLAd) production system.

### MODE FOR CARRYING OUT THE INVENTION

A better understanding of the present disclosure may be obtained through the following examples, which are set forth to illustrate, but are not to be construed as limiting the present disclosure.

Materials and Methods

Reagents, Kits, Experimental Mice, and General Cloning Techniques

All the restriction enzymes, Klenow fragment and HindIII-digested lambda phage DNA were purchased from New England Biolabs (MA, USA). AnyFusion and Pfu polymerase were obtained from Genenmed (Seoul, Korea). Ψ5, which is a kind of Ad5, was as described previously. Chemical reagents were obtained from Sigma (MO, USA). Dulbecco's modified Eagle's medium and fetal bovine serum (FBS) were purchased from Welgene (Gyeongsangbuk-do, Korea) and CellSera (NSW, Australia), respectively. Chemically competent XL-1 Blue and DH10b cells were purchased from RBC (Taipei, Taiwan). The human dystrophin gene, the codon-optimized human huntingtin gene, and mshRs were synthesized by GenScript (NJ, USA). Polymerase chain reaction (PCR) primers and synthetic oligos were obtained from Cosmogenetech (Seoul, Korea). Nucleotide sequence analysis was also performed by Cosmogenetech. The T-blunt PCR cloning kit and LaboPass Tissue Genomic DNA Isolation Kit were purchased from SolGent (Daejeon, Korea) and Cosmogenetech, respectively. Q Sepharose XL and Chelating Sepharose FF resin for column chromatography were obtained from GE Healthcare (IL, USA). The Vivaspin Turbo ultrafiltration spin column (100 kDa cut-off) was purchased from Sartorius (Goettingen, Germany). Benzonase was obtained from Merck (Darmstadt, Germany). Dystrophin-knockout MDX (C57BL/10ScSn-Dmdmdx/J) and wild-type mice (C57BL/10J) were obtained from the Jackson Laboratory (ME, USA). A dystrophin antibody (ab15277) and a huntingtin antibody (sc-47757) were purchased from Abcam (Cambridge, UK) and Santa Cruz Biotechnology (CA, USA), respectively. A β-actin antibody (Abc-2002) was obtained from AbClon (Seoul, Korea). SuperSignal West Pico Chemiluminescent Substrate solution was purchased from Fisher Scientific (NH, USA). HEK293T and HEK293 cells were obtained from ATCC (VA, USA). For the cloning and engineering of DNA sequences, standard DNA manipulation techniques were employed.

### Example 1. Construction of pBest Cloning Shuttle Plasmid

The DNA fragment corresponding to the Kan^{r}˜ColE1 region was synthesized by PCR using the following primer set, with the pGT2 plasmid (SEQ ID NO: 63) serving as a template:

TABLE 1

| SEQ | | | |
|---|---|---|---|
| ID | | | |
| NO: | Name | Sequence (5′->3′) | Note |
| 1 | KanColE1_1IF | GGGCCAAGGATCTGATGGCGC | |
| | | AGGGGA | |
| 2 | KanColE1_1IR | CTTGGCCGCAGCGGCCGAGCA | |
| | | AAAGGCCAGCAAAAGGCCA | |

A DNA fragment encompassing the 5′ homologous stretch, 5′ inverted terminal repeat (ITR), Ψ5 packaging signal, CMV promoter, a multi-cloning site (MCS), an SV40 poly(A) signal, and the 3′ homologous stretch (FIG. 7f) was chemically synthesized by GenScript. This synthetic DNA included an SfiI site at the 5′ end and an XcmI site at the 3′ site. The PCR product and synthetic DNA were ligated together using the SfiI and XcmI restriction sites to generate the pBest plasmid (SEQ ID NO: 64)

### Example 2: Construction of pAdBest_dITR Helper Plasmid

A PCR product was obtained in the same manner as in Example 1, with the exception of using the following primer set:

TABLE 2

| SEQ | | | |
|---|---|---|---|
| ID | | | |
| NO: | Name | Sequence (5′->3′) | Note |
| 3 | KanColE1_2F | CCCGGATCCGCAGTGGGCTTA | |
| | | CATGGCGATAGC | |
| 4 | KanColE1_2R | CCCGTATACATCGATTTAATT | |
| | | AAGAGCAAAAGGCCAGC | |

The PCR product (Kan˜ColE1 region) was digested with BamHI/BstZ17I and ligated with the BstZ17I-BamHI fragment (SEQ ID NO: 65) of the Ψ5 genome. Then, the “asterisk mark to BstZ17I” DNA fragment (SEQ ID NO: 66 FIG. 7a) was prepared by PCR using the primer set and inserted into Ψ5-Left-Arm-1 to produce Ψ5-Left-Arm-2.

TABLE 3

| SEQ | | | |
|---|---|---|---|
| ID | | | |
| NO: | Name | Sequence (5′->3′) | Note |
| 5 | Cl_delE1_F | GGGATCGATTTAAGGGTGGG | |
| | | AAAGAATATATAAG | |
| 6 | BstZ_R | CCCGTATACGGGGACACGGA | |
| | | CAGCCTTTTCGTC | |

Ψ5-Left-Arm-2 is devoid of nucleotides 1-3133 (portion of the 5′ ITR and Ψ packaging signal) of Ad5 (adenovirus type 5, GenBank AC 000008).

To construct Ψ5-Right-Arm-1 (FIG. 7c), the DNA fragment corresponding to the Kan^{r}˜ColE1 region was prepared by PCR using the following primer set

TABLE 4

| SEQ | | | |
|---|---|---|---|
| ID | | | |
| NO: | Name | Sequence (5′->3′) | Note |
| 7 | KanColE1_3F | TTAATTAAGCAGTGGGCTTACA | |
| | | TGGCGATAGC | |
| 8 | KanColE1_3R | CCCGGATCCATCGATTTAATTA | |
| | | AGAGCAAAAGGCCAGC | |

The PCR product was digested with BamHI and ligated with the DNA fragment (SEQ ID NO: 67) corresponding to the BamHI-3′ ITR of the Ψ5 genome.

The partially remaining E3 region in the Ψ5 genome was completely deleted to reduce the genome size of the adenovirus by overlapping PCR using the unique SpeI (27,082) and NdeI (31,089) restriction sites and the primer set of Table 5, with Ad Ψ5 serving as a template.

TABLE 5

| SEQ | | | |
|---|---|---|---|
| ID | | | |
| NO: | Name | Sequence (5′-> 3′) | Note |
| 9 | E3_Spe_F | GGGACTAGTTTCGCGCCCTT | |
| | | TCTCAAATTTAAGC | |
| 10 | delE3_R | GCGGATGGACAGGAACTTAT | |
| | | AACATTCAGTCGTAG | |
| 11 | delE3_F | CTACGACTGAATGTTATAAG | |
| | | TTCCTGTCCATCCGC | |
| 12 | E3_Nde_R | GGGCATATGGATACACGGGG | |
| | | TTGAAGGTATCTTC | |

The resultant Ψ5-Right-Arm-2 construct was devoid of a portion corresponding to nucleotides 27,864-31,000 of Ad5.

Thereafter, the ClaI-BamHI viral DNA (SEQ ID NO: 69) was cleaved out from Ψ5-Left-Arm-2 and ligated to Ψ5-Right-Arm-2 to construct pAdBest (FIG. 7d).

Removal of the 3′ ITR from pAdBest was carried out via the following steps:

pAdBest was first cleaved with ClaI/EcoRI, and a smaller fragment was isolated and ligated with the following adaptor containing ClaI and EcoRI sites to afford pAdBest_EcoR_Cla.

TABLE 6

| SEQ ID NO: | Name | Sequence (5′->3′) | Note |
|---|---|---|---|
| 13 | Cla_EcoR_S | CGATCCGGAGGCCCTTG | |
| 14 | Cla_EcoR_AS | AATTCAAGGGCCTCCGGAT | |

Subsequently, pAdBest_EcoR_Cla was cut with AvrII/RsrII, and a larger fragment was isolated. Overlapping PCR was performed using the following primer sets, with pAdBest serving as a template.

TABLE 7

| SEQ | | | |
|---|---|---|---|
| ID | | | |
| NO: | Name | Sequence (5′->3′) | Note |
| 15 | Avr_F | CTGCCTAGGCAAAATAGCACCC | |
| 16 | Avr_ | AACGATGTAAGTTTTAGGGCGG | |
| | dITR_R | AGTAACTTGTATG | |
| 17 | Avr_ | GCCCTAAAACTTACATCGTTAA | |
| | dITR_F | TTAAGCAGTGGGC | |
| 18 | Rsr_R | TAGCGGTCCGCCACACCCAGCC | |

The PCR product (SEQ ID NO: 70) was digested with AvrII/RsrII and ligated with the larger fragment of pAdBest_EcoR_Cla cleaved with AvrII/RsrII, resulting in the production of pAdBest_EcoR_Cla_dITR with complete removal of the 3′ ITR from pAdBest. Then, the ClaI-EcoRI fragment of pAdBest was ligated back to the corresponding sites of pAdBest_EcoR_Cla_dITR to construct pAdBest_dITR (SEQ ID NO: 71) (FIG. 7e).

### Example 3. Construction of pGLAd Genome Plasmid

PCR was performed using the following primer set, with pAdBest serving as a template:

TABLE 8

| SEQ | | | |
|---|---|---|---|
| ID | | | |
| NO: | Name | Sequence (5′->3′) | Note |
| 19 | KanColE1_ | GGTTGGCGCGCCCTACGTC | |
| | 4F | ACCCGCCCCGTTCCCAC | |
| 20 | KanColE1_ | CCCGAGCTCAAACTACATA | |
| | 4R | AGACCCCCACCTTAT | |

The PCR product (SEQ ID NO: 72) thus obtained was cut with SacI/AscI and then ligated to the following adaptor to produce pAdBestGL1 (FIG. 8a).

TABLE 9

| SEQ | | | |
|---|---|---|---|
| ID | | | |
| NO: | Name | Sequence (5′->3′) | Note |
| 21 | Sac_Pst_Avr_ | CTTAACCTGCAGATCCTCCTA | |
| | Asc_S | GGTTTTTGG | |
| 22 | Sac_Pst_Avr_ | CGCGCCAAAAACCTAGGAGGA | |
| | Asc_AS | TCTGCAGGTTAAGAGCT | |

To remove the unique ClaI site from pAdBestGL1, pAdBestGL1 was cleaved with ClaI, filled-in with Klenow, and then self-ligated, which resulted in pAdBestGL1_dCla. Both pAdBestGL1 and pAdBestGL1_dCla were digested with SacI/AvrII and ligated with a synthetic HpaI-site-containing adaptor (FIG. 8b), which generated pAdBestGL2_wtCla and pAdBestGL2, respectively.

TABLE 10

| SEQ | | | |
|---|---|---|---|
| ID | | | |
| NO: | Name | Sequence (5′->3′) | Note |
| 23 | Sac_Hpa_ | CGGGCGGCGACCTCGCGGGTTAACCGTC | |
| | Avr_S | CTTTAAAAAAGTCGTTTCTGCAAGCTC | |
| 24 | Sac_Hpa_ | CTAGGAGCTTGCAGAAACGACTTTTTTA | |
| | Avr_AS | AAGGACGGTTAACCCGCGAGGTCGCCGCC | |
| | | CGAGCT | |

Then, HpaI-digested pAdBestGL2 was mixed with HindIII-digested lambda phage DNA (arrowhead, FIG. 8c) and subjected to in vitro homologous annealing (iHoA) to construct pAdBestGL3. pAdBestGL3 was cleaved with SacI/ApaI or ApaI/AvrII and ligated with a SacI/ApaI adaptor (FIG. 8d) or an ApaI/AvrII adaptor (FIG. 8e) to produce pAdBestGL4_3H and pAdBestGL4_5H, respectively.

TABLE 11

| SEQ | | | |
|---|---|---|---|
| ID | | | |
| NO: | Name | Sequence (5′->3′) | Note |
| 25 | SacI/ | CATGGTTCCAAAATGCCCCTTAACCGGGT | |
| | ApaI_S | TGGGCC | |
| 26 | SacI/ | CAACCCGGTTAAGGGGCATTTTGGAACCA | |
| | ApaI_AS | TGAGCT | |

TABLE 12

| SEQ | | | |
|---|---|---|---|
| ID | | | |
| NO: | Name | Sequence (5′->3′) | Note |
| 27 | ApaI/ | CATGGTTCCAAAATGCCCCTTAACCGGGTTC | |
| | AvrII_S | | |
| 28 | ApaI/ | CTAGGAACCCGGTTAAGGGGCATTTTGGAAC | |
| | AvrIL_AS | CATGGGCC | |

pAdBest4_3H_2dCla was constructed by consecutively removing the ClaI sites from pAdBestGL4_3H. In detail, pAdBestGL4_3H was cleaved with ClaI, filled-in with Klenow, self-ligated, and transformed into Dam−/− bacterial cells. The resultant pAdBestGL4_3H_dCla construct was cut again with ClaI, filled-in with Klenow and self-ligated to generate pAdBestGL4_3H_2dCla. Similarly, pAdBestGL4_5H was digested with ClaI, filled-in with Klenow and self-ligated to produce pAdBestGL4_5H_dCla. Then, the SacI-ApaI fragment (5′ half portion) of pAdBestGL4_5H_dCla was transferred to SacI/ApaI-cleaved pAdBestGL4_3H_2dCla to construct pAdBestGL5 (FIG. 8f). To produce pAdBestGL (FIG. 8g), pAdBestGL2_wtCla was cut with HpaI, mixed with SacI/AvrII-digested pAdBestGL5 (FIG. 8f) and subjected to iHoA. Then, pAdBestGL5 was cleaved with ApaI/NsiI and ligated with the SMAR element prepared by PCR using the following primer set, to finally construct pGLAd (FIG. 8h).

TABLE 13

| SEQ | | | |
|---|---|---|---|
| ID | | | |
| NO: | Name | Sequence (5′->3′) | Note |
| 29 | pGLAdSMAR_F | TCTGGGCCCAAATAAACTTA | |
| | | TAAATTGTGAGAG | |
| 30 | pGLAdSMAR_R | CCCATGCATATATTTAAAGA | |
| | | AAAAAAAATTGTA | |

### Example 4. In Vitro Homologous Annealing (iHoA)

iHoA was performed using AnyFusion. The entire procedure followed the manufacturer's instructions with slight modification: incubation was performed for 10 min at 55° C., followed by further incubation for 20 min at room temperature. Then, the reaction mixture was transformed into chemically competent XL-1 Blue or DH10b cells and spread over an antibiotic-containing agar plate

### Example 5. Generation of GLAd.LacZ and GLAd3.LacZ

The pGLAd_LacZ or pGLAd3_LacZ plasmid (10 μg) was cut with the Pad restriction enzyme, and Pad activity was heat-inactivated. This PacI-linearized pGLAd_LacZ or pGLAd3_LacZ genome plasmid and 30 μg of the pAdBest_dITR helper plasmid were co-transfected into HEK293T cells plated in a 100 mm culture dish using the calcium phosphate precipitation method. After 6 hours of incubation, the culture medium (10% FBS) was changed to a fresh medium (5% FBS). Forty-eight hours later, the transfected cells and media were harvested (in this step, the culture medium was saved as the viral medium), and the cells were resuspended in 1 ml of fresh culture medium (5% FBS) and disrupted through three cycles of freezing and thawing (the cleared lysate was referred to as the viral lysate). The viral lysate was harvested and used to infect HEK293 cells (recombinant GLAd.LacZ and GLA3.LaZ viruses cannot induce lytic cell death in treated HEK293 cells because the GLAd virus alone cannot be amplified in HEK293 cells). Forty-eight hours after treatment, the cells were stained for LacZ expression.

### Example 6. LacZ Staining

The culture medium was removed from the HEK293 cells, and the cells were fixed with fresh fixation solution (2% formaldehyde/methanol and 0.1% glutaraldehyde in PBS) for 2 min at room temperature. After two careful washes with PBS, the cells were incubated with staining solution [1 mg/ml X-gal, 2 mM magnesium chloride (MgCl₂), 5 mM potassium ferri-cyanide, and 5 mM K ferro-cyanide in PBS) at 37° C. until LacZ staining was evident

### Example 7. Purification of Genomic DNA from Mouse Tail

Genomic DNA was purified from the tail of a C57BL/6J mouse using the LaboPass Tissue Genomic DNA Isolation Kit. The entire procedure followed the manufacturer's instructions with slight modification. Briefly, the mouse tail (2×2 mm piece) was incubated with lysis buffer containing proteinase K until the tissue was completely lysed. The sample was mixed with 100% ethanol and passed through a mini spin column. Bound genomic DNA was thoroughly washed with two different wash buffers and eluted with distilled water.

Cloning of Mouse E-Cadherin Intron 2 Region as pGLAd3 Genome Backbone

Genomic DNA purified from the mouse tail was used as a PCR template. The primer sets and sequences are described in Table 20. PCR amplification of the F1, F2, F3, F4, or F5 fragment was performed for 45 cycles (30 seconds at 95° C., 30 seconds at 60-65° C., 4 min at 72° C.) with each primer set using Pfu polymerase. Each PCR product was cloned into the T-Blunt vector of the T-Blunt PCR cloning kit. The resultant T-F1, T-F2, T-F3, T-F4, and T-F5 products (FIG. 12b) were sequenced for verification.

### Example 9. Construction of pGLAd3 Genome Plasmid

Overlapping PCR was carried out for 50 cycles (30 seconds at 95° C., 30 seconds at 62° C., 60 seconds at 72° C.) using the primer sets N-F/N-R and C-F/C-R (Table 20), with the genomic DNA from the mouse tail serving as a PCR template. The resultant NC fragment PCR product, containing sites for restriction enzymes such as BspEI, XhoI, and NsiI, was subjected to iHoA with pGLAd cut with SacI/AvrII (FIG. 12b) to produce pGLAd_NC. Next, pGLAd_NC was cleaved with NsiI/XhoI and ligated with the annealed synthetic Nsi-Xho fragment (Table 12b) to construct pGLAd_NC_PU, which was the final recipient harboring the F12345 fragment. In parallel, T-F1_dPac was generated by removing the unique Pad site of T-F1 with PacI, blunting with Klenow and self-ligation. T-F2_SMAR was prepared following the ligation of SfiI/SalI-cleaved T-F2 with the SMAR element prepared by PCR using the SMAR-F/SMAR-R primers (table 20), with pGLAd serving as a template. Then, T-F12 was generated by transferring the NotI/RsrII-cut T-F1_dPac fragment to NotI/RsrII-cleaved T-F2_SMAR. The construction of F-T34 was carried out following the transfer of the T-F4 fragment, which was processed with cutting with PvuI, blunting with Klenow, heat-inactivating and additionally cutting with PciI, to the T-F3 that was obtained through processing via cutting with SpeI, blunting with Klenow, heat-inactivating and additionally cutting with PciI. T-F345 was produced by ligating the MluI-XbaI fragment of T-F5 with T-F34 cut with SpeI/MluI (the SpeI site can be ligated to the XbaI site). T-F12345 was generated by transferring the SalI-RsrII fragment of T-F12 to the T-F345 cut with SalI/RsrII. Ultimately, pGLAd3 was constructed utilizing iHoA between PstI-cut pGLAd_NC_PU and XhoI-cut T-F12345. In every step of the construction procedure, sequencing was performed for verification

### Example 10. Construction of pBest4 Cloning Shuttle Plasmid

The pCAG portion of the plasmid was prepared by PCR using the following primer set and then cutting the PCR product with AseI and BsaI.

TABLE 14

| SEQ | | | |
|---|---|---|---|
| ID | | | |
| NO: | Name | Sequence (5′->3′) | Note |
| 31 | pCAG_F | GTTATTAATAGTAATCAATTAGG | |
| 32 | pCAG_R | CTTGGGTCTCCCTATCGCCCGCCGC | |
| | | GCGCTTCGCTTTTTATAGG | |

The β-globin intron region was also prepared by PCR with the following primers and the cutting the PCR product with BsaI and HindIII.

TABLE 15

| SEQ | | | |
|---|---|---|---|
| ID | | | |
| NO: | Name | Sequence (5′->3′) | Note |
| 33 | bGint_F | GGCGATAGGGAGACCCAAGCTGG | |
| | | TGAGTTTGGGGACCC | |
| 34 | bGint_R | GGGAAGCTTGGGTCCCCTGTAGG | |
| | | AAAAAGAAGAAGGCATGAAC | |

The pBest cleaved with AseI/HindIII was ligated with the prepared PCR products to construct the pBest4 shuttle plasmid (SEQ ID NO: 75)

### Example 11. Plaque Assay

HEK293 cells (80-90% confluency) plated in a 100 mm culture dish were treated with Ad.LacZ (first-generation Ad as a positive control; 1×10³ infectious viral particles), GLAd3.LacZ (1.12×10⁷-1.80×10⁷ BFU) or the cell lysate prepared from HEK293T cells transfected with the helper plasmid alone (Table 18). Twenty-four hours later, the culture medium was aspirated, carefully overlaid with 12 ml of a sterilie culture medium containing agarose (0.3%) and incubated in a CO₂ incubator for 10-15 days. A 10 ml aliquot of the diluted MTT solution was added to the agarose layer and incubated for 5 hours. Viral plaques were counted on a light box.

### Example 12. Construction of pAd5pTP Expression Plasmid

The pTP gene of adenovirus type 5 (Ad5) was prepared by PCR using the following primer set, with the Ad Ψ5 DNA serving as a template:

TABLE 16

| SEQ | | | |
|---|---|---|---|
| ID | | | |
| NO: | Name | Sequence (5′->3′) | Note |
| 35 | Ad5pTP_F | GGGAAGCTTACCATGGCCTTGAGCG | |
| | | TCAACGATTGCGCGCGCCTGACC | |
| 36 | Ad5pTP_R | GGCGAATTCCTAAAAGCGGTGACGC | |
| | | GGGCGAGCC | |

The bold portion in SEQ ID NO: 21 is the HindIII site.

The bold portion in SEQ ID NO: 22 is the EcoRI site.

PCR was performed with 40 cycles (30 seconds at 95° C., 30 seconds at 60° C., 120 30 seconds at 72° C.). The PCR product thus obtained was digested with HindIII and EcoRI and cloned into the pLV_XL plasmid prepared by cleavage of pLV_VSVG_XL with HindIII and EcoRI to construct pAd5pTP (SEQ ID NO: 76) expression plasmid.

### Example 13. Large-Scale Production of Recombinant GLAd

To produce recombinant GLAd at a large scale, continuous amplification was employed as demonstrated in the standard method for conventional GLAd production. In brief, HEK293T cells (50-70% confluency) plated in a 100 mm dish were transfected with a mixture of 10 μg of pGLAd3_LacZ (linearized with Pad and heat-inactivated), 30 μg of pAdBest_dITR, and 2.5 μg of pAd5pTP using the calcium phosphate precipitation method. After 6 hours of incubation, the culture medium (10% FBS) was changed to fresh medium (5% FBS). Forty-eight hours later, the transfected cells were harvested, resuspended in 1 ml of fresh culture medium (5% FBS) and disrupted through three cycles of freezing and thawing to rescue the GLAd3. LacZ virus (P0 seed GLAd). For the first round of amplification (P1), HEK293T cells (50-70% confluency) plated in a 100 mm dish were transfected with a mixture of 45 μg of pAdBest_dITR and 3.75 μg of pAd5pTP using the calcium phosphate precipitation method. After 6 h of incubation, the culture medium (10% FBS) was changed to fresh medium (5% FBS) containing P0 seed GLAd. Forty-eight hours later, the transfected cells and media were harvested (in this step, the culture medium was saved as the viral medium), and the cells were resuspended with 1 ml of fresh culture medium (5% FBS) and disrupted through three cycles of freezing and thawing (the cleared lysate was referred to as the viral lysate). The viral lysate was harvested and combined with the viral medium (total 10 ml) and used to infect HEK293T cells (plated in 10×150 mm dishes) for the next round of amplification (P2) (see FIG. 3). Additional rounds of amplification (P3, P4, P5 and so on) can be carried out to increase the GLAd production scale. The flow chart for each round of amplification, the obtained viral titers, the number of plates needed and the required amounts of the helper and pAd5pTP plasmids are described in detail in FIG. 3.

### Example 14. Amplification of Adenovirus and RCA Contaminant Generated During GLAd Production

Unlike GLAd, adenovirus and RCA can replicate in HEK293 cells. Thus, HEK293 cells were infected with Ad. LacZ (positive control) or GLAd3.LacZ (P3, 3×10⁹ BFU; P4 or P5, 1×10⁸ BFU), and the potential contaminant adenovirus and RCA in GLAd3.LacZ were allowed to replicate during one (P4 or P5), approximately two (positive control), or three (P3) rounds of amplification. Because an MOI (multiplicity of infection) of GLAd3.LacZ that is too high, resulting in overexpression of LacZ, can kill infected cells, two 150 mm dishes were used for the initial infection of GLAd3.LacZ (P3, 3×10⁹ BFU). Benzonase was employed to degrade the helper plasmid that was continuously used for the preparation of P3, P4, or P5 GLAd3.LacZ (the helper plasmid contains the target gene for PCR-based analysis; the viral DNA packaged into the capsid shell is resistant to Benzonase). To examine its effects on the infectivity of adenovirus and RCA, Benzonase treatment was also applied to the positive control (Ad.LacZ). For each round of amplification, cells were infected with the corresponding virus and then harvested after 72 h. These cells were resuspended in a volume of 1 ml (for a 100 mm dish) or 2.5 ml (for each 150 mm dish) and disrupted via three cycles of freezing and thawing (the cleared lysate was referred to as viral lysate). Each viral lysate was used for further amplifications, and the cell lysate and culture medium were finally harvested together. During these amplification processes, CPE (cytopathic effect) was examined under a microscope (for the entire workflow, see FIGS. 4a and 4c).

### Example 15. Analysis of Adenovirus and RCA Contaminant by PCR

The serially amplified samples from HEK293 cells (FIGS. 4a and 4c) were first heat-treated for 10 min at 95° C. (if this step is omitted, endogenous cellular DNase degrades the spiked Ad5 DNA) and then analyzed by PCR for N-terminal DNA of the fiber gene, which is present in both adenovirus and RCA but not in the GLAd genome or HEK293 cells. Ad5 DNA (10 μg, Ad Ψ5 DNA) was used as a positive control for PCR. Ad.LacZ samples were subjected to PCR before or after 100× dilution, in which only 500 virus particles are contained in the sample. GLAd3. LacZ samples were PCR-amplified in the presence or absence of spiked Ad5 DNA (10 μg). PCR was carried out for 40 cycles (30 seconds at 95° C., 30 seconds at 60° C., 30 seconds at 72° C.) in the presence of Pfu polymerase using the following primer set, and the results were analyzed by agarose gel electrophoresis (FIGS. 4b and 4d):

TABLE 17

| SEQ | | | |
|---|---|---|---|
| ID | | | |
| NO: | Name | Sequence (5′->3′) | Note |
| 37 | AdSFiber_F | CGCGCAAGACCGTCTGAAGATACC | |
| 38 | AdSFiber_R | GGCCTGATGTTTGCAGGGCTAGC | |

### Example 16. Construction of the pGLAd4 Genome Plasmid

The pGLAd3 contains two BssHII restriction sites (FIG. 14). Treatment of pGLAd3 with BssHII and self-ligation resulted in pGLAd4 (SEQ ID NO: 77), which decreased in the length of plasmid from 26,597 bp (pGLAd3) to 16,392 bp (pGLAd4). After completion, the BssHII site was sequenced for verification.

### Example 17. Construction of Huntingtin mshR Expression Plasmid

The template for mshR expression, which was confirmed to be fully functional, was described previously. Based on these conserved sequence and structural characteristics, the corresponding DNAs were synthesized (Table 21) and cloned into the pGT2 plasmid (SEQ ID NO: 63) using the BamHI and EcoRI sites.

### Example 18. Knockdown of Endogenous Huntingtin Expression by mshR

HEK293T cells plated in a 100 mm culture dish were transfected with 15 μg of pGT2, pGT2-mshR1, pGT2-mshR2, or pGT2-mshR3. Forty-eight hours later, untreated control and transfected cells were harvested and subjected to western blotting analysis for endogenous huntingtin expression.

### Example 19. Western Blotting

Whole-cell lysates prepared using RIPA lysis buffer were resolved in an SDS gel and transferred to a nitrocellulose membrane. The membrane was blocked with Blotto A solution (TBST, 5% milk) for 1 hour at room temperature and further incubated with Blotto B solution (TBST, 1% milk) containing the primary antibody (1:500-1000) at 4° C. overnight. After one 5 min wash with TBST (TBS, 0.05% Tween-20; TBS (Tris-buffered saline): 10 mM Tris-Cl (pH 8.0), 150 mM NaCl), the membrane was incubated with an HRP-conjugated secondary antibody (1:5000-100,000) in Blotto B solution for 2 hours at room temperature. Following two washes (5 min each) with TBST, the membrane was briefly treated with the SuperSignal West Pico Chemiluminescent Substrate solution, and the resulting image was analyzed with a Bio Imaging System.

### Example 20. Construction of the pGLAd4HTTmshR1/3

HTTmshR1 and HTTmshR3 encompassing pCMV, mshR and BGHpA (FIG. 5d) were prepared by PCR using appropriate primer sets (Table 22), with pGT2-mshR1 or pGT-mshR3 serving as templates (FIGS. 5c and 5d). To construct pGLAd4_HTTmshR1/3, the resultant HTTmshR1 and HTTmshR3 constructs were subjected to iHoA with Acc65I-cut pGLAd4 and PciI-cut pGLAd4, respectively (FIG. 5f). After completion, the regions adjacent to the Acc65I and PciI sites were sequenced for verification.

### Example 21. Construction of pGLAd4_coHTT.HTTmshR1/3 and pGLAd4_coHTT(R).HTTmshR1/3

The full-length codon-optimized synthetic huntingtin gene (9.4 kb) containing Q22 was cloned into the pBest4 shuttle plasmid in different orientations. In an approach previously demonstrated as a standard procedure, pBest4_coHTT and pBest4_coHTT(R) were cut with PmeI and subjected to iHoA with ClaI-cleaved pGLAd4_HTTmshR1/3. This iHoA process produced the pGLAd4_coHTT.HTTmshR1/3 or pGLAd4_coHTT(R). HTTmshR1/3 construct. Successful completion was sequenced for verification.

### Example 22. Production of Recombinant GLAd4.coHTT.HTTmshR1/3 and GLAd4.coHTT(R).HTTmshR1/3 Viruses

The same procedure used for producing the recombinant GLAd.LacZ and GLAd3.LacZ viruses was applied.

### Example 23. Purification of Recombinant GLAd4.Dys Virus

The recombinant GLAd4.Dys virus was produced as shown in FIG. 3 and purified from a P3 preparation. For purification, two column-based chromatography methods were employed as reported previously, with slight modification. Briefly, the cell lysate and the harvested culture medium were combined (P3), treated with Benzonase, filtered and subjected to Q Sepharose column chromatography as a first step of purification. After washing, the bound virus was eluted and diluted with buffer and loaded onto a Zn-chelated chromatography column. The column was thoroughly washed, and the bound virus was eluted. Buffer change to a formulation buffer and concentration of virus were simultaneously carried out utilizing Vivaspin Turbo ultrafiltration spin column. The formulation buffer was described previously.

### Example 24. Determination of GLAd4.Dys Viral Particles

The purified recombinant GLAd4.Dys virus was serially diluted using virus lysis buffer [0.1% SDS, 10 mM Tris-Cl (pH 7.4), 1 mM EDTA] and incubated for 10 min at 56° C. with gentle shaking. Then, the OD₂₆₀ was determined and subjected to the calculation of the virus particle concentration using the following equation:

Virus particles (VP/ml)=(OD₂₆₀)×(virus dilution factor)×(1.1×10¹²) [Equation]

In this calculation, the blank solution consisted of a mixture of a virus lysis buffer and a virus formulation buffer. The extinction coefficient was used as established:

OD₂₆₀ unit=1.1×10¹² virus particles/ml. [Extinction coefficient]

### Example 25. Animal Study

All animal experiments were conducted according to the protocol (KNU2018-0134) approved by the Institutional Animal Use and Care Committee (IAUCC) of Kyungpook National University (Daegu, Korea). Eight-week-old male wild-type control mice (C57BL/10J) and dystrophin-knockout MDX mice (C57BL/10ScSn-Dmdmdx/J) were housed under 12-hour light-dark cycles and given water freely in accordance with the Kyungpook National University Animal Facility regulations. The focal gastrocnemius muscles of the MDX mice were injected intramuscularly with PBS (n=3) or with 50 μl of recombinant GLAd4.Dys virus (4×10¹⁰ particles) (n=3). Four weeks later, muscle tissues were biopsied and subjected to analysis.

### Example 26. Immunofluorescence Staining

The immunofluorescence staining of dystrophin was performed as described previously³⁰ with slight modification. Biopsied muscle tissues were fixed at 4° C. overnight with freshly prepared 4% PFA in PBS. Then, the tissues were incubated with 5% sucrose in PBS at 4° C. for 6 h and further incubated with 20% sucrose in PBS at 4° C. overnight. The processed tissues were embedded in OCT, frozen by dipping in liquid nitrogen-chilled isopentane, and stored at −70° C. Four-micron-thick cross-sections were produced, then placed in PBS for 10 min and washed with PBST (0.1% Triton X-100 in PBS) three times for 10 min each at room temperature. The tissue sections were blocked with 10% horse serum in PBST at 4° C. overnight, incubated with a dystrophin antibody (1:100 in blocking buffer) at 4° C. overnight, and then washed with PBST three times for 10 min each at room temperature. Finally, the tissue sections were incubated with a TRITC-conjugated secondary antibody (1:100 in blocking buffer) at 4° C. overnight and washed with PBST three times for 10 min each at room temperature. The stained tissue sections were covered with mounting medium containing DAPI and analyzed under a confocal microscope.

### Results

Construction of pAdBest_dITR Helper Plasmid and pGLAd Genomic Plasmid and Use thereof for Producing GLAd

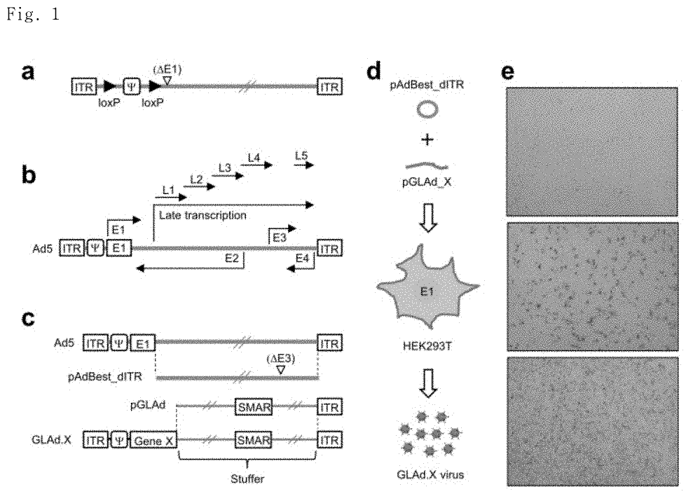

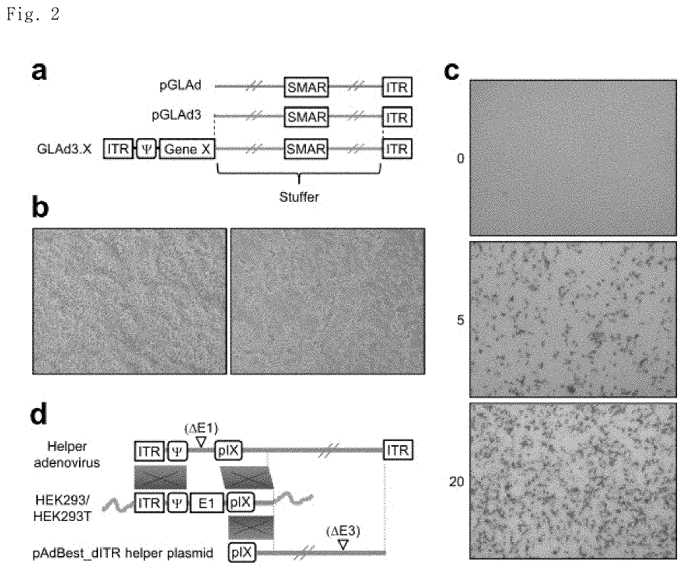

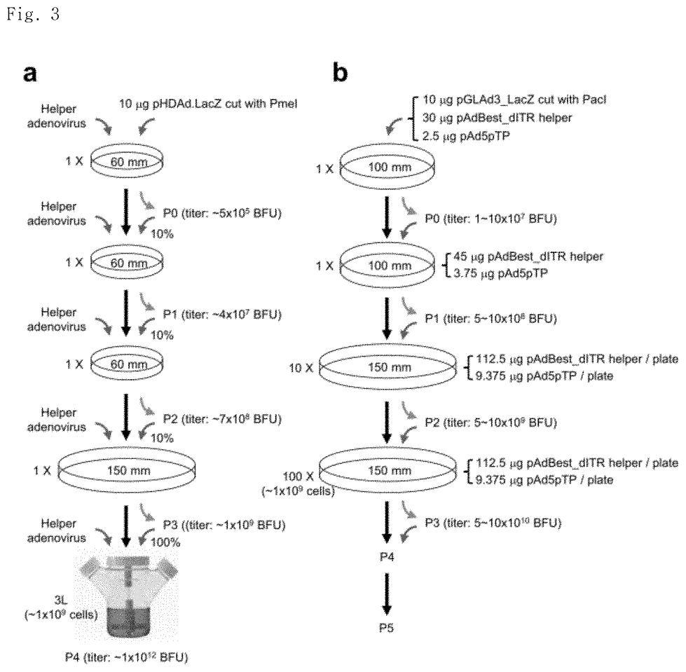

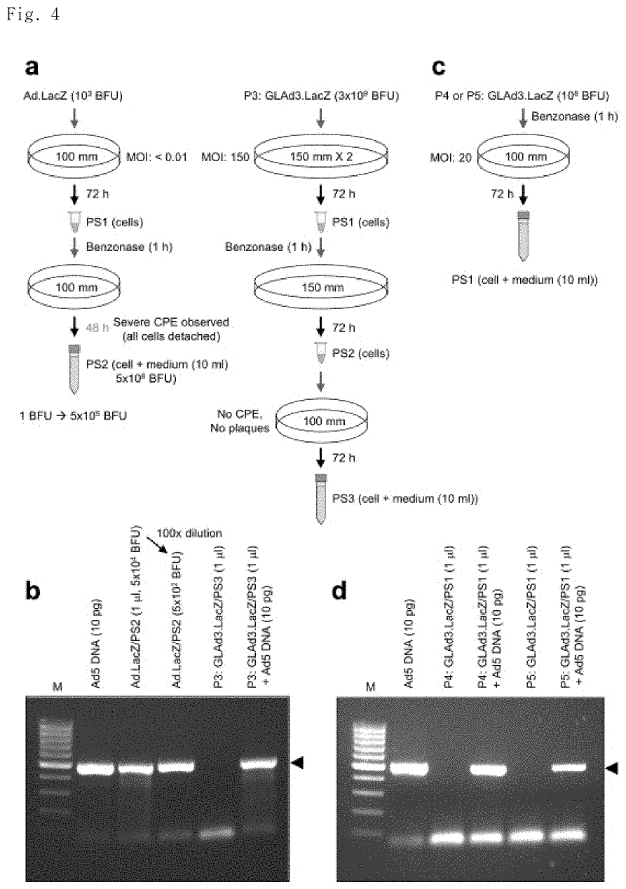

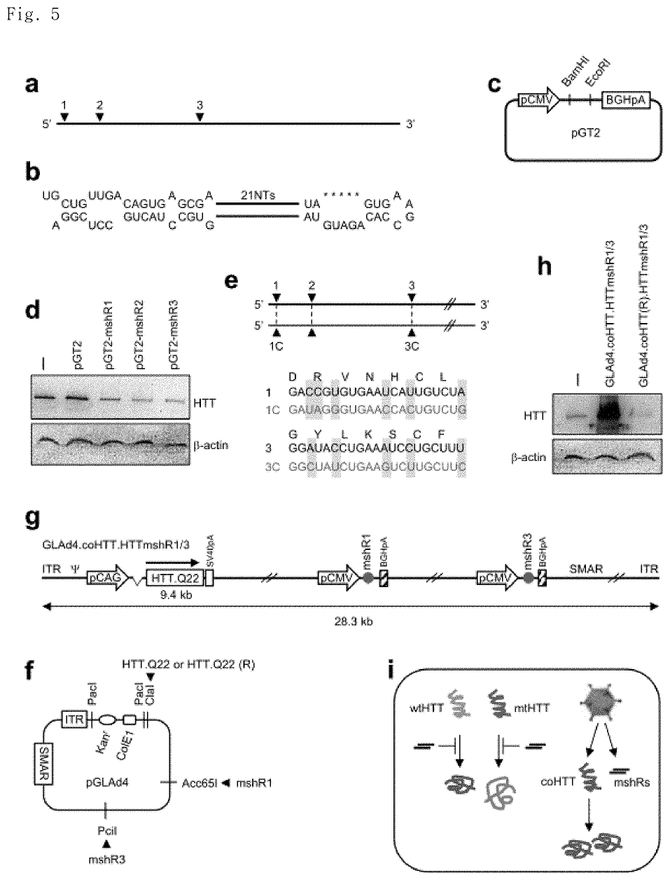

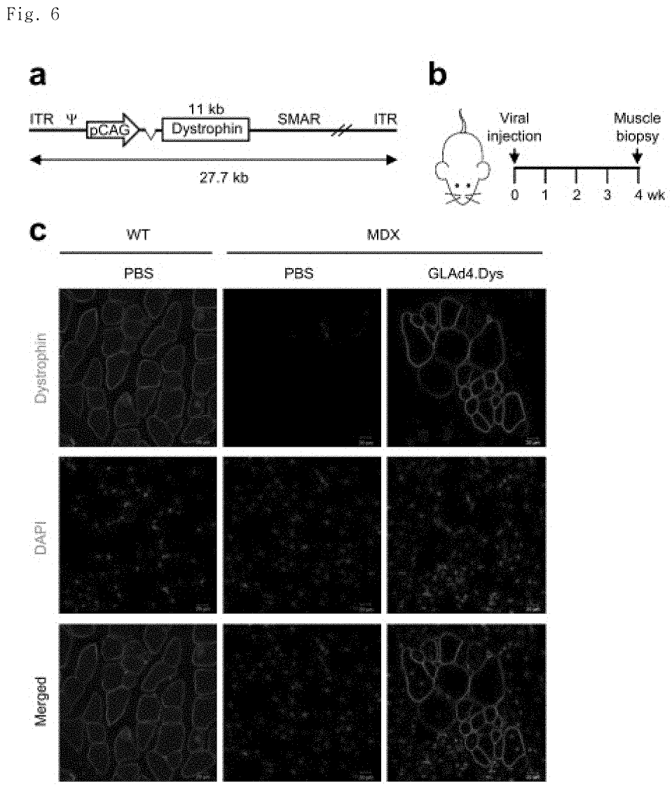

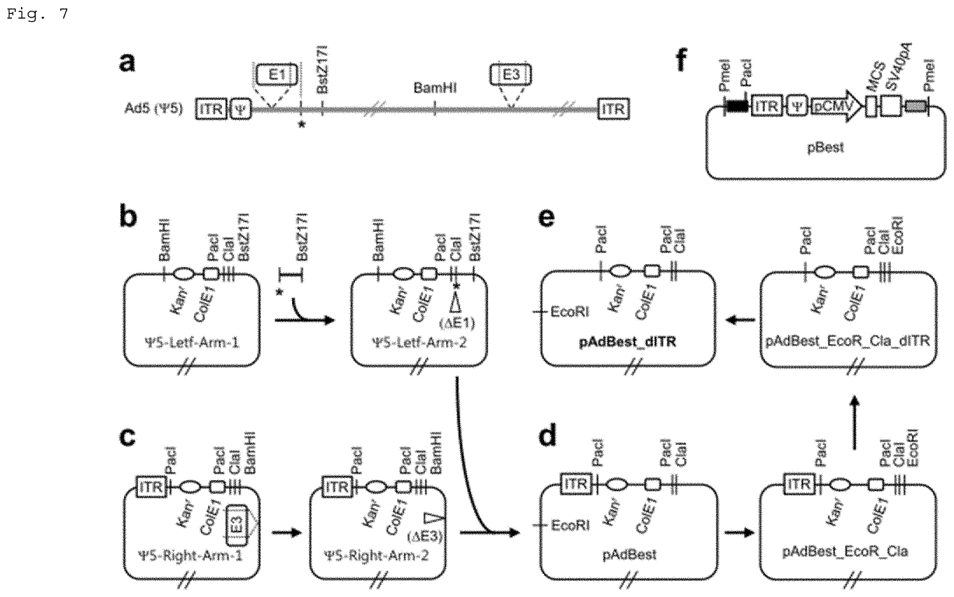

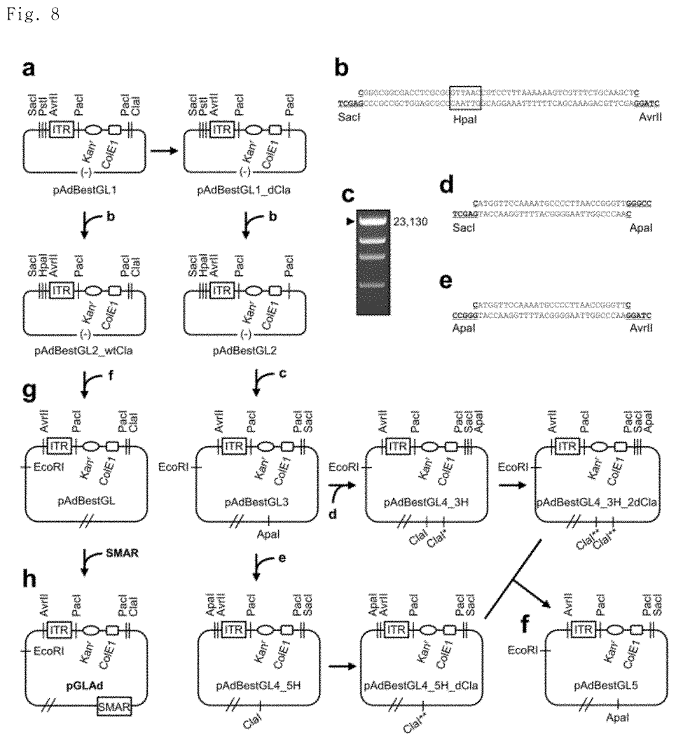

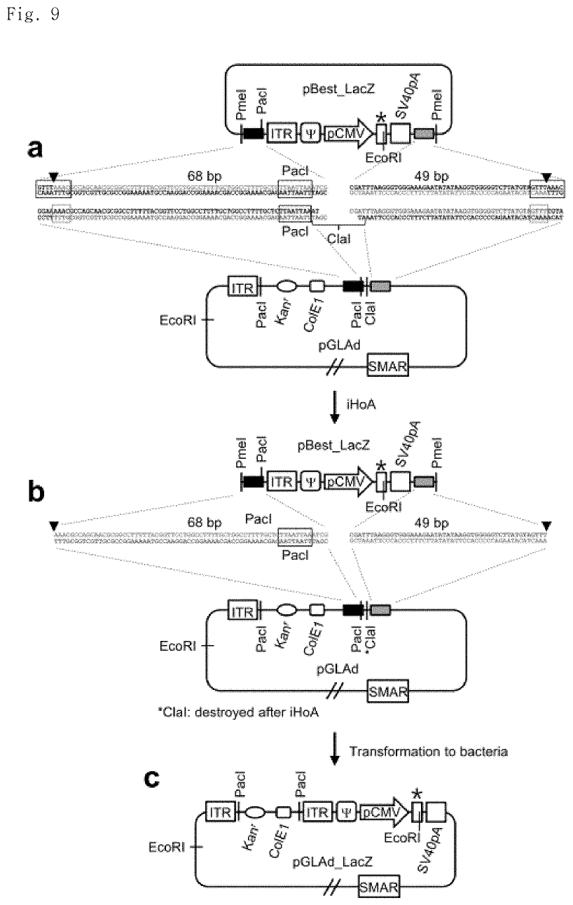

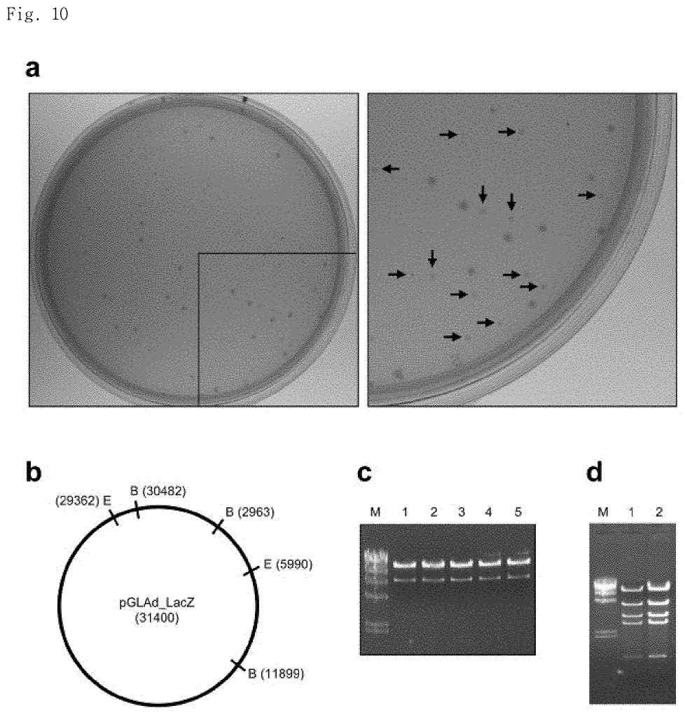

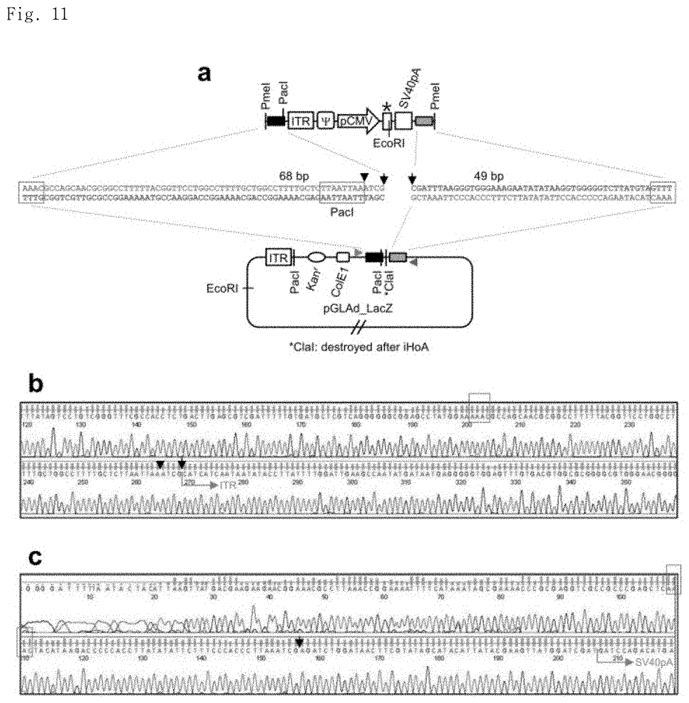

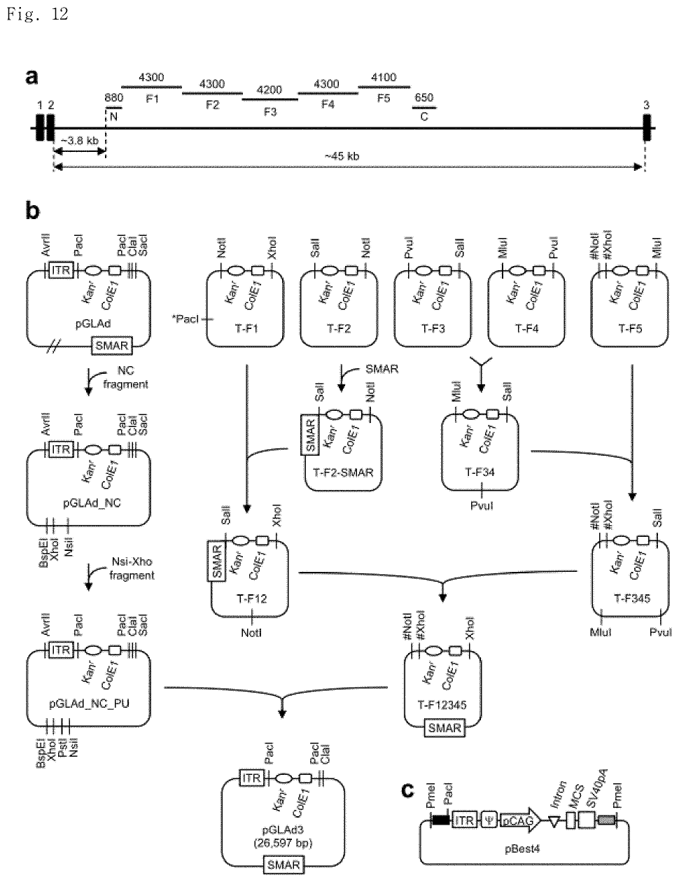

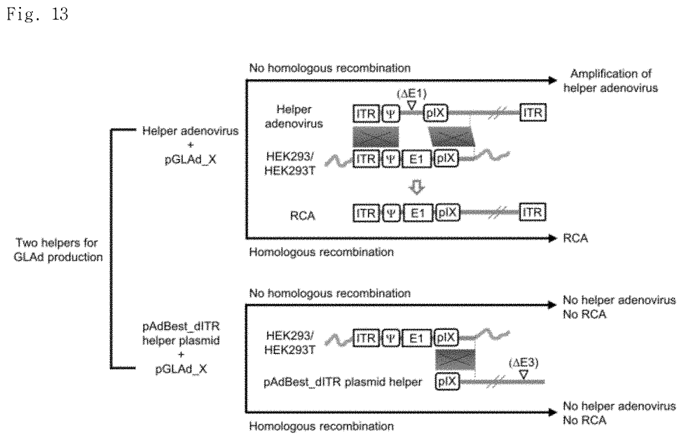

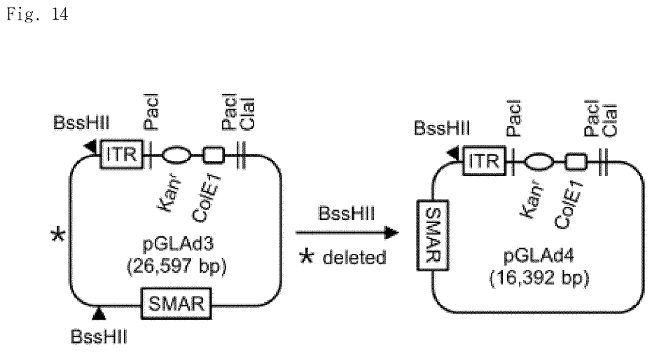

Currently, the most commonly used helper adenovirus contains loxP sites flanking the Ψ packaging signal21,22 (FIG. 1a). Its structural characteristics allow this particular adenovirus to efficiently produce recombinant GLAd with reduced contamination of adenovirus in Cre-expressing packaging cells. Nevertheless, contamination of helper adenovirus is unavoidable even in this sophisticated production system. Moreover, homologous recombination between the helper adenovirus and the E1 region present in packaging cells generates RCA, although RCA has not been intensively analyzed in this system. The present inventors hypothesized that the conversion of the helper adenovirus into a helper plasmid following the deletion of the region involved in homologous recombination might prevent the generation of both adenovirus and RCA contaminants in GLAd preparations. To achieve this goal, the present inventors attempted to generate the helper plasmid that supplies all viral proteins for GLAd packaging but is securely unpackageable into active viral particles, resulting in no generation of RCA.

The helper adenovirus-free recombinant GLAd production system of the present inventors requires two independent plasmids: one serving as a helper for GLAd packaging and the other as the GLAd genome.

As the helper plasmid, the present inventors constructed pAdBest_dITR (˜31 kb) (FIG. 7). Based on the overlapping transcription units and multiple transcripts governed by a single promoter (FIG. 1B), the Ψ5 genome25, a derivative of Ad5 (FIG. 7a), was manipulated to obtain the helper plasmid. The helper plasmid contains neither ITRs nor Ψ packaging signal (FIG. 1c), both of which are essential for virus packaging. Thus, this plasmid only serves as a helper but is securely unpackageable into active viral particles. During this manipulation, the 5′ ITR and Ψ packaging signal were transferred to pBest, a shuttle plasmid (FIG. 7f) that is used for transgene cloning.

As the GLAd genome plasmid, the present inventors constructed pGLAd (FIG. 1c). This pGLAd is composed of stuffer DNA from lambda phage and only the 3′ ITR of Ad5. The scaffold matrix attachment (SMAR) element was used to stabilize the GLAd genome in cells while enhancing transgene expression (for entire construction processes, see FIG. 8).

Generally, it is a tedious process to insert a transgene into a large plasmid such as the pGLAd genome plasmid (˜27 kb). To facilitate this process, short homologous stretches were added to both pBest and pGLAd when constructing these plasmids. These homologous stretches expedited the transfer of the transgene expression cassette from pBest to pGLAd.

As an example of this process, the LacZ gene was first cloned into pBest, which was then linearized with the rare-cutting enzyme PmeI (FIG. 9a). Simultaneously, pGLAd was cut at the unique ClaI site. When linearized pBest_LacZ and linearized pGLAd were mixed and treated with AnyFusion, a nuclease that can convert double-stranded DNA to single-stranded DNA, both linearized homologous regions were efficiently annealed in vitro (the entire process is referred to as “iHoA” for in vitro homologous annealing) (FIG. 9b). The length of the homologous stretches annealed in vitro results in sufficient stability to transform bacterial cells. The resultant recombinant pGLAd_LacZ plasmid recovered both the ITRs and the Ψ packaging signal, and the ITRs were flanked by rare-cutting Pad restriction sites (FIG. 9c). The entire process is quick and efficient in the transfer of a transgene expression cassette to the pGLAd genome plasmid. Moreover, in the transformation step, the bacterial cells containing the correct pGLAd_LacZ plasmid formed colonies that were markedly smaller in size than those of other undesired clones (FIG. 10a). This visually observable characteristic accelerated the screening process (FIGS. 10b to 10d). After the completion of this process, the junctions of the homologous stretches of the resultant recombinant plasmid were sequenced. The nucleotide sequences perfectly matched the homologous stretches (FIG. 11), indicating that the iHoA process operated as designed.

After the construction of both the pAdBest_dITR helper plasmid and the recombinant pGLAd_LacZ genome plasmid, the present inventors tested the two-plasmid-based recombinant GLAd production system. HEK293T cells were transfected with the pAdBest_dITR helper plasmid and the PacI-linearized pGLAd_LacZ genome (FIGS. 1d and 1e). Co-transfected HEK293T cells successfully produced recombinant GLAd.LacZ virus (FIG. 1e), demonstrating that all the constructs and processes involved in the production of recombinant GLAd worked properly as designed.

Construction of New Genome Plasmid pGLAd3

Interestingly, the nature of the stuffer in GLAd has been shown to negatively affect transgene expression. In particular, a lambda DNA stuffer showed this undesirable characteristic, although this finding is controversial. Nevertheless, under the initial conditions, the present inventors utilized lambda DNA to quickly examine the validity of the designed plasmid construction schemes and to determine whether the helper plasmid-based GLAd production system would function properly as expected. Since the GLAd production system operated as designed, the present inventors prepared a new stuffer to reduce concern about the decreased expression of transgenes in the presence of lambda stuffer. The present inventors cloned genomic fragments from the mouse E-cadherin intron 2 region (FIG. 12a) and generated pGLAd3 as a new GLAd genome plasmid (FIGS. 2a and 12b). The present inventors also constructed another cloning shuttle plasmid, pBest4 (FIG. 12c), which was upgraded with a strong CAG promoter and an intron. Similar to the original system, this new system tested with the LacZ gene also effectively produced recombinant GLAd3.LacZ virus (Table 18, FIGS. 2b and 2c) with a cytopathic effect (CPE) (FIG. 2b), indicating that all the elements and processes, such as pCAG, the intron, iHoA, and GLAd packaging, performed correctly as designed. In sharp contrast, HEK293T cells transfected with the pAdBest_dITR helper plasmid alone did not produce any viral particles (Table 18). Additionally, recombinant GLAd produced by the two-plasmid-based system did not contain any other viral species, such as adenovirus or RCA (Table 18). It is clear that the helper plasmid only provides helper function for GLAd packaging but is securely unpackageable into active viral particles. Furthermore, these results indicate that in accordance with its structural characteristics (FIG. 2d), the helper plasmid does not generate RCA (FIG. 13).

### Large-Scale Production of GLAd

The optimized standard method for conventional large scale GLAd production involves a serial amplification process (FIG. 3a). Every round of this process requires freshly added helper adenovirus since GLAd can be amplified only in the presence of the helper adenovirus. Seed GLAd is repeatedly amplified over 4-6 rounds prior to the final round of amplification in large-scale cell culture (˜1×10⁹ cells). This entire amplification process routinely produces ˜1×10¹² BFU (˜1000 BFU/cell; BFU, blue-forming units determined by LacZ staining)²². This relatively high yield of GLAd is attributed to the use of a replicable helper adenovirus that can supply a sufficient amount of viral proteins for GLAd packaging and amplification.

In an attempt to produce GLAd at a large scale utilizing the helper plasmid, the present inventors followed the standard amplification procedure established for conventional large-scale GLAd production, although the helper plasmid cannot replicate in GLAd packaging cells. The present inventors have already shown successful production of seed GLAd (˜2×10⁷ BFU/100 mm dish) by transfecting the PacI-cut GLAd genome plasmid and the helper plasmid (Table 18). This seed GLAd was well amplified by the helper plasmid, and its levels were scaled up (data not shown), even though the amplification efficiency was not yet optimized.

In previous studies of other researchers, it was shown that the additional expression of adenoviral proteins such as E1A, pTP, and IVa2, either individually or in combination, increases adenovirus production. The present inventors tested these adenoviral proteins individually and in combination to improve amplification efficiency in GLAd production. The additional expression of only precursor terminal protein (pTP) increased GLAd production (˜4-fold) (data not shown). Furthermore, the present inventors observed that a greater amount of helper plasmid (45 μg/100 mm dish rather than 30 μg/100 mm dish) resulted in a higher yield in GLAd production (in P1 and thereafter, not in P0; data not shown).

For each amplification procedure, the pAdBest_dITR helper plasmid and the pTP-expressing plasmid were co-transfected into 293T packaging cells, similar to the conventional amplification procedure utilizing helper adenovirus. Through optimization, the present inventors successfully established a standard procedure for the helper plasmid-based large-scale GLAd production (FIG. 3b). P3 routinely produced 5×10¹⁰-1×10¹¹ BFU (50-100 BFU/cell), which is only a 10-20-fold-lower yield compared to the conventional large-scale production method utilizing replicable helper adenovirus (for example, conventional method: new method=1-2×10¹¹ BFU: 1×1010 BFU). P5 is expected to produce 5×10¹²-1×10¹³ BFU from 1×10¹¹ cells.

### Absence of Adenovirus and RCA Contaminant Generation During GLAd Production

Preventing contamination of adenovirus and RCA in GLAd preparation is crucial for considering the clinical application of GLAd. The present inventors have demonstrated that GLAd can be produced by the transfection of the PacI-cut GLAd genome plasmid and the helper plasmid, and none of the three independent GLAd preparations contained any adenovirus and/or RCA (Table 18). Nevertheless, the present inventors analyzed adenovirus and RCA more intensively again in GLAd preparations (P3, P4, or P5) (FIG. 3) whose scales were large enough and, thus, might be used to produce the clinical materials in the future.

Adenovirus (Ad.LacZ) used as a positive control was efficiently amplified in HEK293 cells (FIG. 4a). One infectious viral particle (1 BFU) generated ˜5×10⁵ BFU in less than two complete rounds of amplification. This robust amplification activity of adenovirus caused a severe CPE, which was easily observable with the naked eye. These results suggest that even a single infectious contaminant adenovirus (or RCA) particle in GLAd preparations results in numerous viral particles after a few rounds of amplification. The present inventorse analyzed P3 GLAd3.LacZ (FIG. 3) containing 3×10⁹ BFU (FIG. 4a). Following infecting HEK293 cells with this GLAd, the amplification of adenovirus and RCA, if any, was allowed for three consecutive rounds. These amplification conditions were sufficient for 1 BFU to generate at least 5×10⁵ BFU, causing a severe CPE in HEK293 cells in the final round. However, any CPE could not be observed in infected cells. Accordingly, any adenoviral species (either adenovirus or RCA) could not be detected in the PCR-based analysis (FIG. 4b). These data indicate that the preparation of P3 GLAd (at least 3×10⁹ BFU) did not contain any adenovirus and/or RCA contaminants.

Additionally, the present inventors analyzed the presence of adenovirus and RCA contaminants in P4 and P5 GLAd3.LacZ (1×10⁸ BFU for each). P4 and P5 GLAd were prepared utilizing P3 and P4 GLAd, respectively, as described (FIG. 3). HEK293 cells were infected with P4 or P5 GLAd pretreated with Benzonase, which removes the helper plasmid but not the capsid-protected adenovirus or RCA, and allowed the continued amplification of any potential adenovirus and RCA for additional rounds (P4: one round from P3; P5: two rounds from P3) (FIG. 4c). Similar to P3 GLAd, it was impossible to detect any adenoviral species (either adenovirus or RCA) in the PCR-based analysis (FIG. 4d). Taken together, the data clearly demonstrate that the helper plasmid-based GLAd production system does not generate adenovirus and/or RCA as undesirable contaminants.

### New GLAd as Efficient In Vitro and In Vivo Gene Delivery Vector

Following all the successes described above, the present inventors attempted to test the gene delivery activity of there recombinant GLAd in vitro and in vivo. The present inventors chose huntingtin (9.4 kb) and dystrophin (11 kb) as target transgenes, as it is very difficult for other viral vectors to deliver these large genes and gene therapy has long been pursued as a treatment option for the associated diseases [Huntington's disease (HD) and Duchenne muscular dystrophy (DMD)].

For proper packaging, GLAd genome size from the 5′ ITR to the 3′ ITR should be within the range of 27-37.8 kb, which is 75-105% of the original genome size. Thus, the present inventors first converted the pGLAd3 genome plasmid to pGLAd4 (FIG. 148) to reduce its size because the length of pGLAd3 harboring large genes is near the upper length limit for virus packaging.

Huntington's disease, which is inherited in a dominant fashion, is a fatal neurodegenerative disease caused by a poly-CAG (a codon for glutamine) repeat expansion in huntingtin gene⁴⁰. Huntintun's disease patients possess one mutant copy of this gene, and the disease conditions can be ameliorated when the expression of mutant huntingtin is inhibited by an antisense oligonucleotide or RNAi. Although the normal function of huntingtin in adult nerve cells remains unknown, it is important to note that huntingtin is essential for early embryonic development (huntingtin knockout causes embryonic lethality in mouse). Additionally, overexpression of wild-type huntingtin has been shown to reduce the cellular toxicity of mutant huntingtin. These results suggest that a more appropriate recombinant GLAd for testing is to deliver the RNAi and huntingtin gene sequence together concurrently rather than delivering the huntingtin gene alone.

For RNAi, the present inventors established miRNA-based shRNAs (mshRs hereinafter) instead of conventional shRNAs to knock down the expression of huntingtin since mshRs have been proven to be safer than shRNAs for the knockdown of huntingtin. The present inventors chose three target sites⁴⁸ (FIG. 5a) that have been confirmed to be functional and constructed mshR expression constructs using synthetic mshRs (FIG. 5b and Table 21) and pGT2 plasmids (FIG. 5c). As expected, the transfection of HEK293T cells with each of these constructs resulted in decreased expression of endogenous huntingtin (FIG. 5d). Thus, two mshR expression cassettes for mshR1 and mshR3 were inserted into the pGLAd4 genome plasmid via PCR with appropriate primer sets (Table 22). For the huntingtin gene, it was taken into consideration that the poly-CAG repeats are the only difference between wild-type and mutant huntingtin genes. Thus, a knockdown approach can simultaneously reduce the expression of both endogenous wild type and mutant huntingtin proteins. To decrease concern regarding undesirable toxicity that might result from decreased expression of endogenous wild-type huntingtin, and also to compensate for the decrease in wild-type huntingtin, the present inventors utilized a codon-optimized synthetic huntingtin gene whose expression cannot be attenuated by mshRs (FIG. 5e). Through iHoA, the present inventors successfully transferred a codon-optimized synthetic gene expression cassette in different orientations into the pGLAd4 genome plasmid, in which both the mshR1 and mshR3 expression cassettes had already been inserted (FIG. 5f). Then recombinant GLAd4.coHTT. HTTmshR1/3 (FIG. 5g) and GLAd4.coHTT(R). HTTmshR1/3 viruses were produced.

The GLAd4.coHTT.HTTmshR1/3 virus overexpressed huntingtin, whereas the GLAd4. coHTT(R).HTTmshR1/3 virus inhibited the expression of endogenous huntingtin (FIG. 5h). These research data indicate that the recombinant GLAd successfully delivered both the mshRs and the codon-optimized synthetic huntingtin gene (˜13 kb in total length) simultaneously (FIG. 5i). This recombinant GLAd can potentially be utilized as a gene therapy for the treatment of Huntington's disease and is currently being investigated for clinical application.

In addition to recombinant GLAd delivering the large huntingtin gene, the present inventors have successfully constructed recombinant GLAds for many other small-sized transgenes, such as factor IX (R338L Padua mutant, for hemophilia B), glucocerebrosidase (GCR, for Gaucher's disease), hexosaminidase A (HEXA, for Tay-Sachs disease), hypoxanthine phosphoribosyltransferase 1 (HPRT1, for Lesch-Nyhan syndrome), iduronate-2-sulfatase (IDS, for Hunter syndrome), methyl-CpG-binding protein 2 (MECP2, for Rett syndrome), and survival of motor neuron 1 (SMN1, for spinal muscular atrophy (SMA)). Each of these recombinant GLAds efficiently overexpressed the transgene in vitro (data not shown) and is also currently under investigation for possible clinical applications.

For in vivo study, the present inventors produced a recombinant GLAd that delivers the dystrophin gene (˜11 kb), a target for DMD. First the human dystrophin gene was cloned into pBest4 and a recombinant pGLAd4 Dys plasmid were constructed through iHoA, followed by producing the recombinant GLAd4. Dys virus (FIG. 6a). Then the GLAd4.Dys virus were injected into the focal gastrocnemius muscle of a dystrophin-knockout MDX mouse. Four weeks later (FIG. 6b), the muscle tissue at the injected site was biopsied and analyzed for dystrophin expression (FIG. 6c). The recombinant GLAd4.Dys virus evidently expressed dystrophin in the target tissue. Although improvement of the disease condition was unobservable (unlike humans, MDX mice do not show manifestations of the disease), the data indicate that the GLAd4.Dys virus maintained the expression of dystrophin in the target tissue for at least 4 weeks.

### DISCUSSION

An ideal in vivo gene delivery vector for gene therapy is recommended to exhibit the following characteristics: absence of random integration into the host genome (eliminating concern about insertional mutagenesis), no expression of viral proteins (requiring gutless viral genome, thus resulting in limited host immune response), broad tropism, high transduction efficiency in transgene delivery. and a sufficient cargo capacity for transgenes. Among the many gene delivery vectors currently available, GLAd is the only one that exhibits all of these recommended features.

Surprisingly, however, GLAd has only been used in animal studies so far, and no clinical applications have been attempted, thus, no clinical data for GLAd are currently available14-20. It is believed that this unexpected consequence is associated with the safety concerns raised by adenovirus, which is required for GLAd packaging and further amplification as a helper in the conventional GLAd production process, remaining as a contaminant in prepared GLAd. An additional hurdle to overcome for the clinical use of GLAd is the RCA contaminant²¹ generated during conventional GLAd production procedures or large-scale preparation of helper adenovirus.

The commonly used replication-incompetent first-generation adenovirus is highly immunogenic and can cause cellular toxicity in host organisms20. Thus, this virus has been widely used as a gene delivery vector for anticancer therapy, in which its immunogenic activity provides an adjuvant function and helps to remove tumor tissues more effectively through cooperation with the delivered therapeutic anti-cancer gene. In contrast, adenovirus is harmful in gene therapy, especially for the treatment of inherited genetic diseases requiring long-term transgene expression, since the host immune response caused by adenovirus can result in decreased or short-term transgene expression and toxicity in the patient. In this regard, it is important to note that the best result regarding adenovirus contamination, even in the most advanced helper adenovirus-based GLAd production system, is 0.01%, which corresponds to 1×10⁶ out of 1×10¹⁰ viral particles.

Regarding safety measures, compared with the replication-incompetent first-generation adenovirus (the helper adenovirus used for GLAd production is usually a first-generation adenovirus), RCA is more dangerous. Unlike the first-generation adenovirus, which is devoid of E1, thus allowing replication only in E1-expressing packaging cells such as HEK293 and HEK293T cells, RCA can replicate autonomously in any cell types following its infection. In particular, when gene therapy contaminated with RCA is administered to an immune-suppressed (or immune-weakened) patient, RCA can easily replicate by itself, which could result in fatal toxicity in the patient. Furthermore, the E1 protein expressed from RCA can support the robust replication/amplification of the replication-incompetent adenovirus by complementing E1 deficiency of this adenovirus. Thus, the Food and Drug Administration (FDA) heavily controls RCA and requires that RCA particles should be quantified in every batch of clinical adenovirus (and possibly also GLAd), and a single clinical dose should contain less than one infectious RCA particle in 3×10¹⁰ adenoviral particles. Therefore, it is clear that for the clinical use of GLAd, helper adenovirus should be replaced with a new helper and that a new GLAd production system completely free of concerns about adenovirus and RCA contaminants should be established, as demonstrated for the three plasmid-based AAV production system⁴⁹, a standard procedure for preparing clinical AAV.

In the present disclosure, the present inventors report a novel helper plasmid for GLAd production for the first time. The helper plasmid does not contain any ITRs and Ψ packaging signal, both of which are essential for viral packaging. Additionally, unlike helper adenovirus, the helper plasmid possesses only a single homologous region [nucleotides 3034-5015 (1482 bp) of adenovirus type 5, based on GenBank AC_000008] for homologous recombination, which can occur in HEK293 or HEK293T GLAd packaging cells. These structural characteristics not only eliminate the possibility of the conversion of the helper plasmid to active viral particles but also inhibit the generation of RCA during GLAd production. Actually, the present inventors were unable to detect any adenovirus or RCA contaminants even in the large-scale production of GLAd and in high-passage GLAd prepared through consecutive amplifications. The data thus obtained clearly indicate that the new helper plasmid-based GLAd system exclusively produces target recombinant GLAd completely free of adenovirus and RCA contaminants, which therefore significantly decreases the safety concerns raised by these contaminant viruses.

Although the helper plasmid of the present disclosure cannot replicate in GLAd packaging cells, its helper function for GLAd packaging and further amplification is adequate to produce a sufficient amount of recombinant GLAd. In large-scale GLAd production, the system of the present disclosure exhibited a moderately reduced efficiency compared with a conventional helper adenovirus-based system. Nevertheless, the system allowed large-scale GLAd production with no difficulty. In drug development, safety is generally far more important than production yields. Such a consideration fully justifies the somewhat disadvantageous characteristics regarding the production yield of the GLAd system of the present disclosure.

The recombinant GLAd of the present disclosure effectively delivered many transgenes in vitro and in vivo. As expected, the GLAd efficiently transferred large genes such as huntingtin (9.4 kb) and dystrophin (11 kb) or even multiple expression cassettes (˜13 kb in total length) composed of the huntingtin gene and two mshRs. In many studies, the AAV-mediated delivery of the dystrophin gene has been explored, but with a focus only on the mini- or microdystrophin gene, which are smaller versions of the full-length dystrophin gene exceeding the packaging capacity of AAV. It is important to note that the successful GLAd-mediated delivery of the multiple expression cassettes containing of huntingtin genes and two mshRs represents the first demonstration of a possible therapeutic approach for HD. These examples recapitulate a unique characteristic of GLAd regarding its versatility for gene delivery, particularly for the delivery of large genes.

The use of viral vectors in in vivo gene therapy is a double-edged sword. Viral vectors transfer transgenes more efficiently but can cause toxicity in host organisms. Gutless viral vectors such as AAV and GLAd do not contain any viral genes in their genome backbones; hence, they are safer than other viral vectors expressing viral proteins. Nevertheless, capsid protein-mediated toxicity of these vectors is unpreventable, as observed even for AAV, which exhibits severe acute toxicity at a high dose⁵⁴. However, it is possible to significantly decrease concern about virus capsid-mediated toxicity by utilizing a GLAd that is not contaminated with highly immunogenic adenoviral species or by using a significantly reduced amount of GLAd based on the safe delivery procedure demonstrated by balloon occlusion catheter-mediated intervention for GLAd-based liver-directed transgene expression^{18,19}. This procedure exhibits a high safety profile and achieves long-term transgene expression for as long as 7 years. In this context, the work of the present inventors needs to be evaluated, as a new platform for GLAd is presented in regard to contamination of helper adenovirus and RCA for the first time, along with the description of its outstanding characteristics as an in vivo gene delivery vector (Table 19).

In conclusion, the data obtained in the present disclosure clearly demonstrate that the helper plasmid-based system of the present disclosure efficiently produces recombinant GLAd that is free of adenovirus and RCA contaminants. Currently, gene therapy is addressing unmet needs for the treatment of various inherited genetic diseases. In this particular type of gene therapy, delivery vectors play a pivotal role, and it is hopeful that their helper plasmid and GLAd production system of the present disclosure will pave the way for the successful development of future GLAd-based gene therapy.

TABLE 18
GLAd yield and other viruses generated during GLAd production

| | | GLAd yield^{a} | Other |
|---|---|---|---|
| | | (total BFU) | viruses^{b, c} |
| Helper plasmid + | GLAd3.LacZ #1 | 1.20 × 10⁷ | None |
| pGLAd3_LacZ genome^{d} | GLAd3.LacZ #2 | 1.12 × 10⁷ | None |
| | GLAd3.LacZ #3 | 1.80 × 10⁷ | None |
| Helper plasmid^{d} | #1 | N/A | None |
| | #2 | N/A | None |
| | #3 | N/A | None |
| Ad.LacZ^{e} (1 × 10³ used) | | | ~10³ |

FU blue-forming units,
N/A not applicable
^{a}Determined by LacZ staining (total BFU)
^{b}Adenovirus and/or RCA
^{c}Determined by plaque assay
^{d}Transfected into HEK293T cells plated on 100 mm culture dish
^{e}Positive control for plaque assay

TABLE 19
Comparison of AAV and Conventional GLAd with Novel GLAd

| | Conventional | Novel |
|---|---|---|
| | GLAd | GLAd |
| Use of helper adenovirus in | Yes | No |
| production | | |
| Use of helper plasmid in | N/A | Yes |
| production | | |
| Helper adenovirus contamination | Yes | No |
| Replication-competent adenovirus | Yes | No |
| (RCA) contamination | | |
| | AAV | Novel GLAd |
| Delivery capacity for transgene | ~4.5 kb | ~36 kb |
| Transduction efficiency^{a} | +++ (70%) | +++++ (100%) |
| Broad tropism | Yes | Yes |
| Random integration into host genome | Yes ^{b} | No |
| (potential of insertional | | |
| mutagenesis) | | |
| Expression of viral proteins | No | No |
| In vivo acute toxicity by viral | Yes | Yes |
| capsid | | |
| In vivo chronic toxicity | No | No |
| Long-term in vivo transgene | Yes | Yes |
| expression | | |

N/A not applicable
^{a}Relative activity
^{b} Although frequency is low

TABLE 20
Primers used for pGLAd3 backbone cloning

| SEQ | | | |
|---|---|---|---|
| ID | | | |
| NO: | Name | Sequence (5′->3′) | Note |
| 39 | N-F | TCTTATGTAGTTTGAGCTCGGCTTTGGTTAT | |
| | | TTGATGGATTGACC | |
| 40 | N-R | TCCGGAGGGGCTCGAGGCTGGCTTAAAACAG | |
| | | TAACTCAATATG | |
| 41 | C-F | CTCGAGCCCCTCCGGAGGAGTGGCCAGGGCG | |
| | | TTCTGGAGGTAG | |
| 42 | C-R | CGCCAAAAACCTAGGTGGGCCCAGGAAGACT | |
| | | CACTGGTTGG | |
| 43 | Nsi- | TCGAGCTAGACTCTGGGGCTAAAGCTGCAGT | |
| | Xho-S | ATCCATCACACTGGCGGCCGC | |
| 44 | Nsi- | TCGAGCGGCCGCCAGTGTGATGGATACTGCA | |
| | Xho-AS | GCTTTAGCCCCAGAGTCTAGCTCGATGCA | |
| 45 | F1-F | CCTCCTCGAGCTAGACTCTGGGGCTAAAGCA | |
| | | ATGAG | |
| 46 | F1-R | CCCACGCGTGCGGCCGCTTGAGAGGCAGAGG | |
| | | CAAAGGCAAGTG | |
| 47 | F2-F | TCAAGCGGCCGCGTGCTGAGACTAAAGGGAT | |
| | | GTGCTAC | |
| 48 | F2-R | CCCGTCGACCATCATTCTAAGGCCTGCCTGA | |
| | | GCTA | |
| 49 | F3-F | CCCGTCGACGCACTGGCTTTACAGGGGCCGT | |
| | | CTGC | |
| 50 | F3-R | GGGCGATCGGGAAGGAAACCTACCTAGCCTA | |
| | | CAAG | |
| 51 | F4-F | GGGCGATCGGTAGACTAGGATAGCCTCAAAC | |
| | | TCCT | |
| 52 | F4-R | GGGACGCGTGTTAACATACTATCAGAATAGT | |
| | | GATA | |
| 53 | F5-F | GGGACGCGTTGGGTGCAATCTTACCAGAGCC | |
| | | TTAC | |
| 54 | F5-R | ACGGCAGTTCAAAATCAAGTAATAC | |
| 55 | SMAR-F | CGGCCTGGGTGGCCAAATAAACTTATAAATT | |
| | | GTGAGAGAAA | |
| 56 | SMAR-R | CAGGTCGACATATTTAAAGAAAAAAAAATTG | |
| | | TATC | |

TABLE 21
Synthetic DNA used for huntingtin mshR
expression

| SEQ | | | |
|---|---|---|---|
| ID | | | |
| NO: | Name | Sequence (5′->3′) | Note |
| 57 | HTTmshR1_S | GATCCTGCTGTTGACAGTGAGCGA | |
| | | GACCGTGTGAATCATTGTCTA | |
| | | TAGTGAAGCCACAGATGTA | |
| | | TAGACAATGATTCACACGGTC | |
| | | GTGCCTACTGCCTCGGAG | |
| 58 | HTTmshR1_A | AATTCTCCGAGGCAGTAGGCAC | |
| | S | GACCGTGTGAATCATTGTCTA | |
| | | TACATCTGTGGCTTCACTA | |
| | | TAGACAATGATTCACACGGTC | |
| | | TCGCTCACTGTCAACAGCAG | |
| 59 | HTTmshR2_S | GATCCTGCTGTTGACAGTGAGCGA | |
| | | CAGCTTGTCCAGGTTTATGAA | |
| | | TAGTGAAGCCACAGATGTA | |
| | | TTCATAAACCTGGACAAGCTG | |
| | | GTGCCTACTGCCTCGGAG | |
| 60 | HTTmshR2_A | AATTCTCCGAGGCAGTAGGCAC | |
| | S | CAGCTTGTCCAGGTTTATGAA | |
| | | TACATCTGTGGCTTCACTA | |
| | | TTCATAAACCTGGACAAGCTG | |
| | | TCGCTCACTGTCAACAGCAG | |
| 61 | HTTmshR3_S | GATCCTGCTGTTGACAGTGAGCGA | |
| | | GGATACCTGAAATCCTGCTTT | |
| | | TAGTGAAGCCACAGATGTA | |
| | | AAAGCAGGATTTCAGGTATCC | |
| | | GTGCCTACTGCCTCGGAG | |
| 62 | HTTmshR3_A | AATTCTCCGAGGCAGTAGGCAC | |
| | S | GGATACCTGAAATCCTGCTTT | |
| | | TACATCTGTGGCTTCACTA | |
| | | AAAGCAGGATTTCAGGTATCC | |
| | | TCGCTCACTGTCAACAGCAG | |

The nucleotides in bold letters at 5′ and 3′ ends account for adhesive ends for BamHI and EcoRI sites, respectively.

TABLE 22
PCR primers used for cloning huntingtin
mshR into pGLAd3 backbone

| SEQ | | | |
|---|---|---|---|
| ID | | | |
| NO: | Name | Sequence (5′->3′) | Note |
| 63 | pGLAd3_ | ATTTTAAAGGGCCACGGTACGACATT | |
| | 5139ACC_ | GATTATTGAGTAGTTATTAA | |
| | CMV_AF_F | | |
| 64 | pGLAd3_ | TGTGTCCATCCGTGTGGTACCCATAG | |
| | 5139Acc_ | AGCCCACCGCATCCCCAGCA | |
| | BGH_AF_R | | |
| 65 | pGLAd3_ | TTAACCCCACTCCCCACATGGACATT | |
| | 7189Pci_ | GATTATTGACTAGTTATTAA | |
| | CMV_AF_F | | |
| 66 | pGLAd3_ | GCATCTGAACGAAGCACATGCCATAG | |
| | 7189Pci_ | AGCCCACCGCATCCCCAGCA | |
| | BGH_AF_R | | |

Underlined nucleotide sequences are portions homologous to pGLAd3 genome plasmids.

### INDUSTRIAL APPLICABILITY

The present disclosure pertains to a helper plasmid-based gutless adenovirus (GLAd) production method and system.

1. A gutless adenovirus (GLAd) production system, comprising a helper plasmid, a genome plasmid, and a virus packaging cell strain.

2. The gutless adenovirus (GLAd) production system of claim 1, wherein the helper plasmid:
(a) is not an infectious viral particle;
(b) does not undergo conversion into a virus particle;
(c) is free of an inverted terminal repeat (ITR);
(d) is free of an ITR and a Ψ packaging signal; or
(e) comprises one to five plasmids.

3-6. (canceled)

7. The gutless adenovirus (GLAd) production system of claim 1, wherein the genome plasmid:
(a) comprises a 5′ homologous stretch, a 3′ homologous stretch, and a 3′ inverted terminal repeat (ITR);
(b) further comprises an antibiotic-resistant gene;
(c) further comprise an Ori replication origin;
(d) further comprises a stuffer DNA (sDNA);
(e) comprises a GLAd genome portion to be packaged into a capsid of GLAd; or
(f) is a final genome plasmid comprising a transgene to be expressed using GLAd, and elements necessary for transgene expression.

8-10. (canceled)

11. The gutless adenovirus (GLAd) production system of claim 1, wherein the genome plasmid further comprises a stuffer DNA (sDNA), and the stuffer DNA further comprises scaffold/matrix attachment element (SMAR).

12-13. (canceled)

14. The gutless adenovirus (GLAd) production system of claim 1, wherein the system further comprises a cloning shuttle plasmid.

15. The gutless adenovirus (GLAd) production system of claim 14, wherein the cloning shuttle plasmid comprises a 5′ homologous stretch, a 5′ inverted terminal repeat (ITR), a Ψ packaging signal, a promoter, a multi-cloning site (MCS), a poly(A) signal, and a 3′ homologous stretch.

16. The gutless adenovirus (GLAd) production system of claim 15, wherein the cloning shuttle plasmid;
(a) comprises an intron;
(b) further comprises an antibiotic-resistant gene;
(c) further comprises an Ori replication origin; or
(d) comprises a transgene to be expressed using GLAd, and elements necessary for transgene expression.

17-19. (canceled)

20. The gutless adenovirus (GLAd) production system of claim 1, wherein the virus packaging cell strain is a cell strain expressing a protein belonging to an E1 region of adenovirus.

21. The gutless adenovirus (GLAd) production system of claim 1, wherein the system further comprises a pAd5pTP expression plasmid, which optionally comprises the sequence of SEQ ID NO: 76.

22. (canceled)

23. A method for producing gutless adenovirus (GLAd), the method comprising the steps of:
transfecting a final genome plasmid into a virus packaging cell strain; and
transfecting a helper plasmid into the virus packaging cell strain.

24. The method of claim 23, wherein the final genome plasmid is linearized by a restriction enzyme.

25. The method of claim 23, wherein the transfection is carried out by a calcium phosphate precipitation method.

26. The method of claim 23, wherein the method further comprises a step of transfecting a pAd5pTP expression plasmid into a virus packaging cell strain, and optionally the pAd5pTP expression plasmid comprises the nucleotide sequence of SEQ ID NO: 76.

27. The method of claim 23, wherein the helper plasmid:
(a) is not an infectious viral particle;
(b) does not undergo conversion into a virus particle;
(c) is free of an inverted terminal repeat (ITR);
(d) is free of an ITR and a Ψ packaging signal; or
(e) comprises one to five plasmids.

28-31. (canceled)

32. The method of claim 23, wherein the final genome plasmid:
(a) comprises a 5′ inverted terminal repeat (ITR), a Ψ packaging signal, a promoter, an intron, a transgene, a poly(A) signal, a stuffer DNA (sDNA), and a 3′ inverted terminal repeat;
(b) further comprises an antibiotic-resistant gene;
(c) further comprises an Ori replication origin;
(d) comprises a GLAd genome portion to be packaged into a capsid of GLAd; or
(e) comprises a transgene to be expressed using GLAd, and elements necessary for transgene expression.

33-34. (canceled)

35. The method of claim 32, wherein the stuffer DNA further comprises a scaffold/matrix attachment element (SMAR).

36-37. (canceled)

38. The method of claim 23, wherein the virus packaging cell strain expresses a protein belonging to an E1 region of adenovirus.

39. The method of claim 23, wherein the GLAd produced by the production method is free of a contaminant virus species.

40. The method of claim 39, wherein the contaminant virus species is adenovirus or replication-competent adenovirus (RCA).

41. (canceled)

42. A gutless adenovirus, comprising a 5′ inverted terminal repeat (ITR), a LP packaging signal, a promoter, an intron, a transgene, a poly(A) signal, a stuffer DNA (sDNA), and a 3′ inverted terminal repeat (ITR).
